SEQUENCE LISTING (86 sequences)

SEQ ID NO: 1 - DNA, 795 bp, Escherichia coli
  CDS (1)..(795): B-lactamase
atgagtcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgg

SEQ ID NO: 2 - protein, 265 aa, Escherichia coli
MSHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 3 - DNA, 858 bp, Escherichia coli
  CDS (1)..(858): B-lactamase
atgagaattcaacatttccgtgtcgcccttattcccttttttgcggcattttgccttcctgtttttggtcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgg

SEQ ID NO: 4 - protein, 286 aa, Escherichia coli
MRIQHFRVALIPFFAAFCLPVFGHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 5 - DNA, 795 bp, Escherichia coli
  CDS (1)..(795): B-lactamase
atggggcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgatgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcattgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgg

SEQ ID NO: 6 - protein, 265 aa, Escherichia coli
MGHPETLVKVKDAEDQLGARVGYIELDLNSGKILESFRPEERFPMMSTFKVLLCGAVLSRDDAGQEQLGRRIHYSQNDLVEYSPVTEKHLTDGMTVRELCSAAITMSDNTAANLLLTTIGGPKELTAFLHNMGDHVTRLDHWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 7 - DNA, 792 bp, Escherichia coli
  CDS (1)..(792): B-lactamase
atggacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcattgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgg

SEQ ID NO: 8 - protein, 264 aa, Escherichia coli
MDPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDHWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 9 - DNA, 786 bp, Escherichia coli
  CDS (1)..(786): B-lactamase
atgaaagatgattttgcaaaacttgaggaacaatttgatgcaaaactcgggatctttgcattggatacaggtacaaaccggacggtagcgtatcggccggatgagcgttttgcttttgcttcgacgattaaggctttaactgtaggcgtgcttttgcaacagaaatcaatagaagatctgaaccagagaataacatatacacgtgatgatcttgtaaactacaacccgattacggaaaagcacgttgatacgggaatgacgctcaaagagcttgcggatgcttcgcttcgatatagtgacaatgcggcacagaatctcattcttaaacaaattggcggacctgaaagtttgaaaaaggaactgaggaagattggtgatgaggttacaaatcccgaacgattcgaaccagagttaaatgaagtgaatccgggtgaaactcaggataccagtacagcaagagcacttgtcacaagccttcgagcctttgctcttgaagataaacttccaagtgaaaaacgcgagcttttaatcgattggatgaaacgaaataccactggagacgccttaatccgtgccggagcggcatcatatggaacccggaatgacattgccatcatttggccgccaaaaggagatcctgtcggtgtgccggacggttgggaagtggctgataaaactgttcttgcagtattatccagcagggataaaaaggacgccaagtatgatgataaacttattgcagaggcaacaaaggtggtaatgaaagccttaaacatgaacggcaaa

SEQ ID NO: 10 - protein, 262 aa, Escherichia coli
MKDDFAKLEEQFDAKLGIFALDTGTNRTVAYRPDERFAFASTIKALTVGVLLQQKSIEDLNQRITYTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGAASYGTRNDIAIIWPPKGDPVGVPDGWEVADKTVLAVLSSRDKKDAKYDDKLIAEATKVVMKALNMNGK

SEQ ID NO: 11 - DNA, 720 bp, Aequorea victoria
  CDS (1)..(720): mutant green fluorescent protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttctcctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaacctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 12 - protein, 239 aa, Aequorea victoria
MVSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHNLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 13 - DNA, 690 bp, Anemonia majano
  CDS (1)..(690): fluorescent protein
atggctctttcaaacaagtttatcggagatgacatgaaaatgacctaccatatggatggctgtgtcaatgggcattactttaccgtcaaaggtgaaggcaacgggaagccatacgaagggacgcagacttcgacttttaaagtcaccatggccaacggtgggccccttgcattctcctttgacatactatctacagtgttcaaatatggaaatcgatgctttactgcgtatcctaccagtatgcccgactatttcaaacaagcatttcctgacggaatgtcatatgaaaggacttttacctatgaagatggaggagttgctacagccagttgggaaataagccttaaaggcaactgctttgagcacaaatccacgtttcatggagtgaactttcctgctgatggacctgtgatggcgaagaagacaactggttgggacccatcttttgagaaaatgactgtctgcgatggaatattgaagggtgatgtcaccgcgttcctcatgctgcaaggaggtggcaattacagatgccaattccacacttcttacaagacaaaaaaaccggtgacgatgccaccaaaccatgtggtggaacatcgcattgcgaggaccgaccttgacaaaggtggcaacagtgttcagctgacggagcacgctgttgcacatataacctctgttgtccctttctga

SEQ ID NO: 14 - protein, 229 aa, Anemonia majano
MALSNKFIGDDMKMTYHMDGCVNGHYFTVKGEGNGKPYEGTQTSTFKVTMANGGPLAFSFDILSTVFKYGNRCFTAYPTSMPDYFKQAFPDGMSYERTFTYEDGGVATASWEISLKGNCFEHKSTFHGVNFPADGPVMAKKTTGWDPSFEKMTVCDGILKGDVTAFLMLQGGGNYRCQFHTSYKTKKPVTMPPNHVVEHRIARTDLDKGGNSVQLTEHAVAHITSVVPF

SEQ ID NO: 15 - DNA, 696 bp, Zoanthus sp.
  CDS (1)..(696): fluorescent protein
atggctcagtcaaagcacggtctaacaaaagaaatgacaatgaaataccgtatggaagggtgcgtcgatggacataaatttgtgatcacgggagagggcattggatatccgttcaaagggaaacaggctattaatctgtgtgtggtcgaaggtggaccattgccatttgccgaagacatattgtcagctgcctttaactacggaaacagggttttcactgaatatcctcaagacatagttgactatttcaagaactcgtgtcctgctggatatacatgggacaggtcttttctctttgaggatggagcagtttgcatatgtaatgcagatataacagtgagtgttgaagaaaactgcatgtatcatgagtccaaattttatggagtgaattttcctgctgatggacctgtgatgaaaaagatgacagataactgggagccatcctgcgagaagatcataccagtacctaagcaggggatattgaaaggggatgtctccatgtacctccttctgaaggatggtgggcgtttacggtgccaattcgacacagtttacaaagcaaagtctgtgccaagaaagatgccggactggcacttcatccagcataagctcacccgtgaagaccgcagcgatgctaagaatcagaaatggcatctgacagaacatgctattgcatccggatctgcattgccctga

SEQ ID NO: 16 - protein, 231 aa, Zoanthus sp.
MAQSKHGLTKEMTMKYRMEGCVDGHKFVITGEGIGYPFKGKQAINLCVVEGGPLPFAEDILSAAFNYGNRVFTEYPQDIVDYFKNSCPAGYTWDRSFLFEDGAVCICNADITVSVEENCMYHESKFYGVNFPADGPVMKKMTDNWEPSCEKIIPVPKQGILKGDVSMYLLLKDGGRLRCQFDTVYKAKSVPRKMPDWHFIQHKLTREDRSDAKNQKWHLTEHAIASGSALP

SEQ ID NO: 17 - DNA, 696 bp, Zoanthus sp.
  CDS (1)..(696): fluorescent protein
atggctcattcaaagcacggtctaaaagaagaaatgacaatgaaataccacatggaagggtgcgtcaacggacataaatttgtgatcacgggcgaaggcattggatatccgttcaaagggaaacagactattaatctgtgtgtgatcgaagggggaccattgccattttccgaagacatattgtcagctggctttaagtacggagacaggattttcactgaatatcctcaagacatagtagactatttcaagaactcgtgtcctgctggatatacatggggcaggtcttttctctttgaggatggagcagtctgcatatgcaatgtagatataacagtgagtgtcaaagaaaactgcatttatcataagagcatatttaatggaatgaattttcctgctgatggacctgtgatgaaaaagatgacaactaactgggaagcatcctgcgagaagatcatgccagtacctaagcaggggatactgaaaggggatgtctccatgtacctccttctgaaggatggtgggcgttaccggtgccagttcgacacagtttacaaagcaaagtctgtgccaagtaagatgccggagtggcacttcatccagcataagctcctccgtgaagaccgcagcgatgctaagaatcagaagtggcagctgacagagcatgctattgcattcccttctgccttggcctga

SEQ ID NO: 18 - protein, 231 aa, Zoanthus sp.
MAHSKHGLKEEMTMKYHMEGCVNGHKFVITGEGIGYPFKGKQTINLCVIEGGPLPFSEDILSAGFKYGDRIFTEYPQDIVDYFKNSCPAGYTWGRSFLFEDGAVCICNVDITVSVKENCIYHKSIFNGMNFPADGPVMKKMTTNWEASCEKIMPVPKQGILKGDVSMYLLLKDGGRYRCQFDTVYKAKSVPSKMPEWHFIQHKLLREDRSDAKNQKWQLTEHAIAFPSALA

SEQ ID NO: 19 - DNA, 699 bp, Discosoma striata
  CDS (1)..(699): fluorescent protein
atgagttgttccaagagtgtgatcaaggaagaaatgttgatcgatcttcatctggaaggaacgttcaatgggcactactttgaaataaaaggcaaaggaaaaggacagcctaatgaaggcaccaataccgtcacgctcgaggttaccaagggtggacctctgccatttggttggcatattttgtgcccacaatttcagtatggaaacaaggcatttgtccaccaccctgacaacatacatgattatctaaagctgtcatttccggagggatatacatgggaacggtccatgcactttgaagacggtggcttgtgttgtatcaccaatgatatcagtttgacaggcaactgtttctactacgacatcaagttcactggcttgaactttcctccaaatggacccgttgtgcagaagaagacaactggctgggaaccgagcactgagcgtttgtatcctcgtgatggtgtgttgataggagacatccatcatgctctgacagttgaaggaggtggtcattacgcatgtgacattaaaactgtttacagggccaagaaggccgccttgaagatgccagggtatcactatgttgacaccaaactggttatatggaacaacgacaaagaattcatgaaagttgaggagcatgaaatcgccgttgcacgccaccatccgttctatgagccaaagaaggataagtaa

SEQ ID NO: 20 - protein, 232 aa, Discosoma striata
MSCSKSVIKEEMLIDLHLEGTFNGHYFEIKGKGKGQPNEGTNTVTLEVTKGGPLPFGWHILCPQFQYGNKAFVHHPDNIHDYLKLSFPEGYTWERSMHFEDGGLCCITNDISLTGNCFYYDIKFTGLNFPPNGPVVQKKTTGWEPSTERLYPRDGVLIGDIHHALTVEGGGHYACDIKTVYRAKKAALKMPGYHYVDTKLVIWNNDKEFMKVEEHEIAVARHHPFYEPKKDK

SEQ ID NO: 21 - DNA, 678 bp, Discosoma sp.
  CDS (1)..(678): fluorescent protein
atgaggtcttccaagaatgttatcaaggagttcatgaggtttaaggttcgcatggaaggaacggtcaatgggcacgagtttgaaatagaaggcgaaggagaggggaggccatacgaaggccacaataccgtaaagcttaaggtaaccaaggggggacctttgccatttgcttgggatattttgtcaccacaatttcagtatggaagcaaggtatatgtcaagcaccctgccgacataccagactataaaaagctgtcatttcctgaaggatttaaatgggaaagggtcatgaactttgaagacggtggcgtcgttactgtaacccaggattccagtttgcaggatggctgtttcatctacaaggtcaagttcattggcgtgaactttccttccgatggacctgttatgcaaaagaagacaatgggctgggaagccagcactgagcgtttgtatcctcgtgatggcgtgttgaaaggagagattcataaggctctgaagctgaaagacggtggtcattacctagttgaattcaaaagtatttacatggcaaagaagcctgtgcagctaccagggtactactatgttgactccaaactggatataacaagccacaacgaagactatacaatcgttgagcagtatgaaagaaccgagggacgccaccatctgttcctttaa

SEQ ID NO: 22 - protein, 225 aa, Discosoma sp.
MRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERTEGRHHLFL

SEQ ID NO: 23 - DNA, 801 bp, Clavularia sp.
  CDS (1)..(801): fluorescent protein
atgaagtgtaaatttgtgttctgcctgtccttcttggtcctcgccatcacaaacgcgaacatttttttgagaaacgaggctgacttagaagagaagacattgagaataccaaaagctctaaccaccatgggtgtgattaaaccagacatgaagattaagctgaagatggaaggaaatgtaaacgggcatgcttttgtgatcgaaggagaaggagaaggaaagccttacgatgggacacacactttaaacctggaagtgaaggaaggtgcgcctctgcctttttcttacgatatcttgtcaaacgcgttccagtacggaaacagagcattgacaaaatacccagacgatatagcagactatttcaagcagtcgtttcccgagggatattcctgggaaagaaccatgacttttgaagacaaaggcattgtcaaagtgaaaagtgacataagcatggaggaagactcctttatctatgaaattcgttttgatgggatgaactttcctcccaatggtccggttatgcagaaaaaaactttgaagtgggaaccatccactgagattatgtacgtgcgtgatggagtgctggtcggagatattagccattctctgttgctggagggaggtggccattaccgatgtgacttcaaaagtatttacaaagcaaaaaaagttgtcaaattgccagactatcactttgtggaccatcgcattgagatcttgaaccatgacaaggattacaacaaagtaacgctgtatgagaatgcagttgctcgctattctttgctgccaagtcaggcctag

SEQ ID NO: 24 - protein, 266 aa, Clavularia sp.
MKCKFVFCLSFLVLAITNANIFLRNEADLEEKTLRIPKALTTMGVIKPDMKIKLKMEGNVNGHAFVIEGEGEGKPYDGTHTLNLEVKEGAPLPFSYDILSNAFQYGNRALTKYPDDIADYFKQSFPEGYSWERTMTFEDKGIVKVKSDISMEEDSFIYEIRFDGMNFPPNGPVMQKKTLKWEPSTEIMYVRDGVLVGDISHSLLLEGGGHYRCDFKSIYKAKKVVKLPDYHFVDHRIEILNHDKDYNKVTLYENAVARYSLLPSQA

SEQ ID NO: 25 - protein, 881 aa, Saccharomyces cerevisiae
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSIDSAAHHDNSTIPLDFMPRDALHGFDWSEEDDMSDGLPFLKTDPNNNGFFGDGSLLCILRSIGFKPENYTNSNVNRLPTMITDRYTLASRSTTSRLLQSYLNNFHPYCPIVHSPTLMMLYNNQIEIASKDQWQILFNCILAIGAWCIEGESTDIDVFYYQNAKSHLTSKVFESGSIILVTALHLLSRYTQWRQKTNTSYNFHSFSIRMAISLGLNRDLPSSFSDSSILEQRRRIWWSVYSWEIQLSLLYGRSIQLSQNTISFPSSVDDVQRTTTGPTIYHGIIETARLLQVFTKIYELDKTVTAEKSPICAKKCLMICNEIEEVSRQAPKFLQMDISTTALTNLLKEHPWLSFTRFELKWKQLSLIIYVLRDFFTNFTQKKSQLEQDQNDHQSYEVKRCSIMLSDAAQRTVMSVSSYMDNHNVTPYFAWNCSYYLFNAVLVPIKTLLSNSKSNAENNETAQLLQQINTVLMLLKKLATFKIQTCEKYIQVLEEVCAPFLLSQCAIPLPHISYNNSNGSAIKNIVGSATIAQYPTLPEENVNNISVKYVSPGSVGPSPVPLKSGASFSDLVKLLSNRPPSRNSPVTIPRSTPSHRSVTPFLGQQQQLQSLVPLTPSALFGGANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKE

SEQ ID NO: 26 - protein, 202 aa, Saccharomyces cerevisiae
MKALTARQQEVFDLIRDHISQTGMPPTRAEIAQRLGFRSPNAAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWL

SEQ ID NO: 27 - DNA, 17 bp, Unknown Organism
  Description of Unknown Organism Nucleotide sequence that is bound by GAL4
cggnnnncnnnncnccg

SEQ ID NO: 28 - DNA, 14 bp, Unknown Organism
  Description of Unknown Organism Nucleotide sequence that binds LexA
cgaacnnnngttcg

SEQ ID NO: 29 - DNA, 25 bp, Artificial Sequence
  Description of Artificial Sequence Synthetic primer
ctagacgttgacattgattattgac

SEQ ID NO: 30 - DNA, 25 bp, Artificial Sequence
  Description of Artificial Sequence Synthetic primer
tctagagccagtaagcagtgggttc

SEQ ID NO: 31 - DNA, 28 bp, Artificial Sequence
  Description of Artificial Sequence Synthetic primer
gcggccgcccattgcattgggattggtc

SEQ ID NO: 32 - DNA, 26 bp, Artificial Sequence
  Description of Artificial Sequence Synthetic primer
gcggccgccctccaagtctttatctg

SEQ ID NO: 33 - DNA, 28 bp, Artificial Sequence
  Description of Artificial Sequence Synthetic primer
ggtaccggctcgtagagaaatatgaacc

SEQ ID NO: 34 - DNA, 26 bp, Artificial Sequence
  Description of Artificial Sequence Synthetic primer
ctcgagagagactgaatttccctttt

SEQ ID NO: 35 - DNA, 30 bp, Artificial Sequence
  Description of Artificial Sequence Synthetic primer
gcggccgcgaaggaagatagccaaggctta

SEQ ID NO: 36 - DNA, 32 bp, Artificial Sequence
  Description of Artificial Sequence Synthetic primer
gcggccgcttaaagatgccacactgctggtct

SEQ ID NO: 37 - DNA, 26 bp, Artificial Sequence
  Description of Artificial Sequence Synthetic primer
gtcgacacctgtcggctactgggata

SEQ ID NO: 38 - DNA, 26 bp, Artificial Sequence
  Description of Artificial Sequence Synthetic primer
gatatcgtgggactctgtttccagca

SEQ ID NO: 39 - DNA, 6795 bp, Artificial Sequence
  Description of Artificial Sequence Synthetic recombinant DNA vector sequence
aagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgcctgagaaaggaagtgagctgtaaaggctgagctctctctctgacgtatgtagcctctggttagcttcgtcactcactgttcttgactcagcatggcaatctgatgaaatcccagctgtaagtctgcataaattgatgatctattaaacaataaagatgtccactaaaatggaagttttttactgtcatactttgttaagaagggtgagaacagagtacctacattttgaatggaaggattggagctacgggggtgggggtgggggtgggattagataaatgcctgctctttactgaaggctctttactattgctttatgataatgtttcatagttggatatcataatttaaacaagcaaaaccaaattaagggccagctcattcctccactcatgatctatagatctatagatctctcgtgggatcattgtttntctgatccactggaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtgcggccgggccatgcaggccacgacatgataagatacattgatgagtttggacaaaccacaactagaatgcagtgaaaaaaatgctttatttgtgaaatttgtgatgctattgctttatttgtaaccattataagctgcaataaacaagttcttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgttagcttgatttgacagtggctgggggttgcgccgccgggttttataggaagccacagcggccactcgagccataaaaggcaactttaggaacggcggggggtgattggattcgagtcgtttattcaccggccttgccgcacagtgcagcatttttttaccccctctcccctccttttgcgggggaaaaaaaaaaaaaaaaaaaaaaaaggagaagagaaaaaaagcgagcgagagagaaagcgagattgaggaagaggatgaagagtttggcgatgggtgctggttccgtaggcccagatggacaagaatagcccccgcccttgcggacagtatcccattcagtgactcagatcagatcaagcggccgctctagaactagtggatccggataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaaccccgggctgcaggaattctaccgggtaggggaggcgcttttcccaaggcagtctggagcatgcgctttagcagccccgctggcacttggcgcatcacaagtggcctctggcctcgcacacattccacatccaccggtagcgccaaccggctcccttctttggtggccccttcgcgccaccttctactcctcccctagtcaggaagttcccccccgccccgcagctcgcgtcgtgcaggacgtgacaaatggaagtagcacgtctcactagtctcgtgcagatggacaagcaccgctgagcaatggaagcgggtaggcctttggggcagcggccaatagcagcttggctccttcgctttctgggctcagaggctgggaaggggtgggtccgggggcgggctcaggggcgggctcaggggcggggcgggcgcgaaggtcctccggacccggcattctgcacgcttcaaaagcgcacgtctgccgcgctgttctcctcttcctcatctccgggccttcgacctgcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccg

SEQ ID NO: 40 - DNA, 5821 bp, Artificial Sequence
  Description of Artificial Sequence Synthetic recombinant DNA vector sequence
ggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagttgactgtatcgccgggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaa

SEQ ID NO: 41 - DNA, 10075 bp, Artificial Sequence
  Description of Artificial Sequence Synthetic recombinant DNA molecule sequence
ctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtgcggccgggccatgcaggccacgacatgataagatacattgatgagtttggacaaaccacaactagaatgcagtgaaaaaaatgctttatttgtgaaatttgtgatgctattgctttatttgtaaccattataagctgcaataaacaagttcttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgttagcttgatttgacagtggctgggggttgcgccgccgggttttataggaagccacagcggccactcgagccataaaaggcaactttaggaacggcggggggtgattggattcgagtcgtttattcaccggccttgccgcacagtgcagcatttttttaccccctctcccctccttttgcgggggaaaaaaaaaaaaaaaaaaaaaaaaggagaagagaaaaaaagcgagcgagagagaaagcgagattgaggaagaggatgaagagtttggcgatgggtgctggttccgtaggcccagatggacaagaatagcccccgcccttgcggacagtatcccattcagtgactcagatcagatcaagcggccgccagtgtgatggatatctgcagaattccagcacactggcggccgttactagtggatccctctggagctgcctagtgtgggtgtctgctggaaacttctaaaatgtctcggcctatttaattggagaattcactgaagaattaaaggactcagtaaagaagaggtttggtttgttcatgaaagtaagtagcttgtgaaatgctttattttcctgtgactaaaattccaccttgtaattcccaataaggattgcttcaagctatttaatacaaattaaacctgtttcaccatgctcataggaaaatgtcaaagtatcttcattcaccaactgagggctggctcttctttgaaggtagctctgagatttgttttgtaatggagacaaagacttatttaactgttggggagggggaaggaggacttaggccaggcaggcggggatgtgggggacgaaccatgaggggcaatttggctgcctctgaaaaggtccagagctaccttaaaagacaggctttctataacaagtggtcctttcttttcctttctttttttttttgagacagagtctcactctgttgcccaagctggagtgcagtggtgcgatctcagctcactgcaacctccagctcccaggttcaagcaattctcctgcctcagcctcccaagtagctgggattacaggcaaccgctaccatgcccagctaattttttgtatttttaatagagatggggtttcaccatgttggccagggtggtctcaaactcctgacctcaagtgatccacccacctcagcctcccaaagtgctgggattacagacatgagccaccgcgcccagacacagctggctttgtcgcccaggctggaagtgcagtggcacgatctcggctcactgcaagctctgcctcctgggttcacgccattctcctgcctcagcttcccaagtagctgagactacaggcgcccgccaccacgcccggctaattttttgtatttttagtggagatggtgtttcaccatgttagccaggatggtcttgatctcctgacctcgtgatccgcctgtcttggcctcccaaagtgctgggatgacaggcgtgagccaccacgcctggcttacaagtggccctttctaagccccaagatgtcactgtcttttcactgaaggcggaaaaagaatgtaggcatgtttgtctgagagggagggagtagttttaggagaaaggaccaagagcacattttgagtgggaagtaagtggtttccttttctagccttatgatatggtttggctctgtgtccccacccaaatctcatggtgaactgtaatccccagtggtggaggtgaggcctggcgggagggactggatcatgggggtgatccttcatgaatggtttagcaccatcctcttggtgctactctagtgatagcgttctcacaagatctgatgggtgaaaagtgtgtgtctcttgtttttgctcctgccatgcaagacgcctactcccacttcaccttctgctatgagtcaaattccctgaggcctccccagaagcagaagcttcctgtacagccttgtgggccacgagccaattaaacctcttttctttataaattaccaggtctcagggatttctttatagcagactaatacacctcaaagtagggatttattttattattctattttttttttgagatagggtctcgatctgtcacccaggctggagtgcagtggcatgatctcagcttgctgtaacctctgcctcccgggttgaagcaattctcatgcctaagccacccaagtagctggaattacaggtgtgtgccaccctgctcagctaatttttgtatctttagtagagttggggtttctccatgttggccaggctggtatcaaactcctggactcaagcaatctgcccaccttggcctccccagagtgccaggattacaggcatgacccatcttgcctggtcagtttttttttttttaaatccagttaatgctgggtgccatgactcacacctgtaatgccagtgctttgggaggccgaggtgggaagatcacttgaggccaggagtttgagaccagcctgggaaacatctcatctctaccaaaaaacaaaaacaaaacaaaacaacaacaacatcaaacatagccaggtgtggtggtgcacacctgtagtcccagctccttgggaggctgaggcaggaggattgcttgagcccaggagttcaaggctgcagtgaactatgattgcaccactgcattccagcctgggcaacagagcaagaacctatctctatttaagaaaaaatgctcctaagaaatcatcttagtacttggtaagctattaaggttttttttaagtgtattaattacatagccagtataataacttagttgctaagagctttgaggtggctccaagcaagaagaaaggagttgttgttaagctacagctcagagtctcagaatgaactgtttccatcttcgctgtcctttgcttaactgtgggataaatataattttctcaacattcaacacatgtgtccttaatcgtcctctacgacactggttccatgttgccaagagaacccagaacagccgcaaacacctactggattgttactggcctttcctgagaggatgacccgggctttgaccttgttcatgttgaagttcttcaagtaggcctcgtagattctcttggccagagatttgagatctgcagtttcagaatcttctatgtcatgttcacaggtaagaatttctgctttcagttttgctttctcagatcttggcattcgtccggtacccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatggtggcaagcttaagtttaaacgctagaactagtggatccggataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaaccccgggctgcaggaattctaccgggtaggggaggcgcttttcccaaggcagtctggagcatgcgctttagcagccccgctggcacttggcgcatcacaagtggcctctggcctcgcacacattccacatccaccggtagcgccaaccggctcccttctttggtggccccttcgcgccaccttctactcctcccctagtcaggaagttcccccccgccccgcagctcgcgtcgtgcaggacgtgacaaatggaagtagcacgtctcactagtctcgtgcagatggacaagcaccgctgagcaatggaagcgggtaggcctttggggcagcggccaatagcagcttggctccttcgctttctgggctcagaggctgggaaggggtgggtccgggggcgggctcaggggcgggctcaggggcggggcgggcgcgaaggtcctccggacccggcattctgcacgcttcaaaagcgcacgtctgccgcgctgttctcctcttcctcatctccgggccttcgacctgcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgcctgagaaaggaagtgagctgtaaaggctgagctctctctctgacgtatgtagcctctggttagcttcgtcactcactgttcttgactcagcatggcaatctgatgaaatcccagctgtaagtctgcataaattgatgatctattaaacaataaagatgtccactaaaatggaagttttttactgtcatactttgttaagaagggtgagaacagagtacctacattttgaatggaaggattggagctacgggggtgggggtgggggtgggattagataaatgcctgctctttactgaaggctctttactattgctttatgataatgtttcatagttggatatcataatttaaacaagcaaaaccaaattaagggccagctcattcctccactcatgatctatagatctatagatctctcgtgggatcattgtttntctgatccactggaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccg

SEQ ID NO: 42 - DNA, 8971 bp, Artificial Sequence
  Description of Artificial Sequence Synthetic recombinant DNA vector sequence
gtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccgggtaccgcagtggggatgtctcataatggtaagtaaacagtcatcaccatatactttattattctcattatagctgccagaccagtggacactaaagccattgccaaaaatgtgtacagtttttccaccaaatgccagaatttagaatattgcatggcgataaaacatttctcttttaggtcagtgtttttaaagttttattatagaacctttctctctgtggttgggcatctgccatgaggagaaaagagacttgaaaaatctgggggattatgggaaaaactttagtgttagaacaaatataatgatcatcatgattaatttttatattgcctcatgcattaaaaacagaattaaaatttacttttgtgtatcagtagtgtcattctgctttaaatgtaagaaaatcctgaagatgtctgggtctgaaactttaaatggcatatgagaatttagattcatttaattttttttttttttttttttttgctgtttattatgcaaagacgagtattcgtttccttcacttcaaggaacgtccatgagttgtggaaagggtaaaacaaattatttttatagttctgcaaaacttcaaagccatagaaacataagagaataaaactttctacttttgaaacctgtgtctaaagtcaagcttatgactttgggtgtcaacttaagggaaatgatgtagaagacctgagaaaagacccatgttttcatgatctgtggaactaactcagtaaggtcctggaagacaaacgtgtttgaaaaaataattcttggtcactatttggttctgattggttgcctggctgacaaaataaattttaggtgtaacttgatagtattatatgtgtctagtattaatcagttattttcaatgcactcaaggaataaaaaatgccaacatgatcaaattaaatttgaagtttcatttcatcagctatcctattttttctttcttttgttttcatctctctcaaaactgatctagtaagaaggcagtcttcatctatatgtactctaaagcatctgagaaaattaaatttttgctctatcatgcagatacttggttttcacagtggaaaccatcaagatatttgagagtcactcattgattaataacctaccctttaaaaatagttcacaaaattctatctgtatgattactaatgggtcagttcctctaaaggcaagaatgtattttgaatagagctatcagcagtactgaacaggatggaaatttggtccaactttcctcaacaattgactacatctgctgtatacaactgtagtcatggattgcaaaataatttgctggttcagttccatcagtttcctttttatttcagacctcataaaacataagggcaagcttccaagtcaagggttgtattttaggtgatgcaatcaagttgcaataaacttatttcatcttcatgaagaaatcattgactttgaggtcattatcactgagacagcctaggaaagcatgccagttccgcttttaacactaatttttgcctattaaaaaattatcacttcacacattcatgtaaaatataattcctgttacttacaggcagtgcatacattgatgtaaaatatatttcctgttgcttacaggcattgcatacgtttacttaaaatcaatctgcaattgcttattgtttataggcaaatattatttaaaccccaacctatgtagtatggatggtataattttctcctcccataaatgtcaaagaaagctatcataaacttgataggtcatattttgacttcatatgacaatttattttatttctagagagggtcaatcttgatagggagtgtttgttcagttctagcaaaagcaagaaaatatatgaattaagtgtcatgacatcagagacttaggcagttcaacacttaaaaaattgcaaacataactaagaaaacctcagaggtttttatgggagtttgtttgctgcacagaaaataaacatcagactaacaacaaggttttgattagttttgcctgttagctgtatgaagaaaacatttctaaatacaaagttttctaggaagttgagtcactatttaaaatccattgctttgaccaatgtgaagccaacagggttttcattcatatagaatgcctttttatcaaaagaggctctgagtgttataccaaaatcagcacccagctgttttcagtatgcgcaagaaaacatgaacgtcccccaaagcaaatgtactccattccgtgttaaccttatttctgctccctggtctctgattagtctggttgatgaatgtagcttcccctttggccatgcactcacagctttgaaggaggcttctgaaggaactggtagggaaccctaggggctcttgctaatgttgttggatagtcactgaaatcagtgatcaccatgtcctttcactttgaaaattctatgattctgtataatcaaacaacctaactcactggatttttcacttttcaaccagctattccgtgtcttacagtgctctggtcttcacagataggtaggcacctggcatgtcagttgcctgaatttgaaagttttcacctgtatgttttggtacgataaaaataaaaatgtaatttatatatctgaatcaggtctgtatgttatgatcaattgctcagcaatttcgggcagttggtttgatggttatgtagtaatgtagcctgagagcagaaatacagagcctctgggctagagaaagtataaatggcatcctaggctatgtagggttacagctcttcagaaggaactttcattttcattgtgacacatcgtctacatgttgtagaagaacatagtttcagaattcttccagttagaaacatagtttctcaaatattcactttcagcattgggtagaaaaagtacccaggtgatggactgattgaggatccactagtaacggccgccagtgtgctggaattctgcagatatccatcacactggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctg

SEQ ID NO: 43 - DNA, 29 bp, Artificial Sequence
  Description of Artificial Sequence Synthetic primer
cgggtccccggcgatacagtcaactgtct

SEQ ID NO: 44 - DNA, 30 bp, Artificial Sequence
  Description of Artificial Sequence Synthetic primer
taaagcttgccaccatgaagctactgtctt

SEQ ID NO: 45 - DNA, 10514 bp, Artificial Sequence
  Description of Artificial Sequence Synthetic recombinant DNA vector sequence
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtgcggccgggccatgcaggccagacatgataagatacattgatgagtttggacaaaccacaactagaatgcagtgaaaaaaatgctttatttgtgaaatttgtgatgctattgctttatttgtaaccattataagctgcaataaacaagttgtcgagttacttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgttagcttgatttgacagtggctgggggttgcgccgccgggttttataggaagccacagcggccactcgagccataaaaggcaactttaggaacggcggggggtgattggattcgagtcgtttattcaccggccttgccgcacagtgcagcatttttttaccccctctcccctccttttgcgggggaaaaaaaaaaaaaaaaaaaaaaaaggagaagagaaaaaaagcgagcgagagagaaagcgagattgaggaagaggatgaagagtttggcgatgggtgctggttccgtaggcccagatggacaagaatagcccccgcccttgcggacagtatcccattcagtgactcagatcagtcaagcggccgcgtctaacctaggagcccaccacagacactatactataaccacactgaattttctctcctatctcaaataatcctattcttgttcatgcctcagtgccttataacaagcagatttctcggccagacatggtggcttgcgcctgtaatcccagcactttggccaagtcgggcagctcacttgaggtcaggagtttgaattcagcctgtccaatatggtgaaaccccatctctactaaaaatacaaaaattagctgggcatggtggtgtgtgcctgtagtcccagctactcaggagactgaggcaggagaattgcttgaactcgggaagcagaggttgcagtgagccaagatcatgccattgcactccagcctgggcaacagagcgagactccgtctcaaacaaaacaaaacaaaaacaaaaaaacaagcagatctctcaacttggaatgctgctgcttctccatgccaactggaaaacttctttttcttttagactcaactcaaatatgatcctccctgaagtctctccatctttcctagacacaattagcaaccagttcctctgtgttcaaacagcacttcgtgtgtatgtgtatgtatgtgtatttatcattattattatcacctttgatcactctccctgaaggtaatagccaggcacttagtatagagtaggtatcaacaattgcttattgaaggaataaatgggtgaatgacaactttgcaaagtttcaatttttaatctattgatttgaaagaattattatacattattttaaatatcctttctagttctaatatcttatgtctatagtagttcgacagttactttaaatcagatattaaatgattttcattttacaaagaacagacctgcctttctcaatgtcacaagactattgtagagaatagcagtgaaataagtacatgtgaaaatacttggcaaagtaatagtgaaaatagagctgatctcacaggaatgttttgaaaattaaaacaccgtatgtaaaataggtaatctaatctgtgggcacatatttaaactctcaataaatattaactttgattatttaaaaaatacctggtagcttttttgtgaattctacaagaacctgtacatgattggttgccatttccgtcaaaatgagaaaattttcttctgcactgaattcttcttttaactaaattttaagaaaaaaaccaatgattaaaaagtagataagaaaatgataatataaaaccattagactaaagaaaaactaggtttagatgataaaatttgaaaacccatcttactatttctcagatctttggagggcaattaccattatcaagataggtgggagacttcatactcttgaattgtttaatgttgctgagagtttaaaagtctaacttgaattctcagctctaatcatttcaaagtaacatattcacatataaattatacctccaaggcaataaaaactccagaaaatattaactttaaacccacatattttcagatattatacatacaattttatttgttatttcctgaggcatcctctgtttgttatatgaatccataataaaatgtagaagagtctgttgatctggggtgagttcagttttctcctacactggcaagaaaataaaaggtgttagggaagatgtgtataaagccttctttctatatgtgcattcatctggagggacctaatagcagaaatcataaaaaaaaaaaggcataaatacagcaggaactatgagttctaagagagtgtataatttctttctctgggtcctcaggcaggccatatcacatttcactgaattttcctatctataaaatatgaataacaaaatattccacttgtgtatttcttaggaataaatggtagtctggatatgaagactattgagcccattcatattgaaggtcactgaaagttcaagctttaattataactaatgaaaggttttcatatagtatgactactatgaatactatattcatatactattctacatgaattccacctggtgaagtcatctgagagtattcatgtggaggagggatcaacaaactgtggcccatggaccaaatccattggattttataaagttttattggaacacagtcacactcatttgtttatgtattgtctctgctacttttgcactaaaatggcagagttgagtagctgttgtagataccatgtggccaaagccctttaaagaaaaaaagtttgtcaacccctaaaatagagagtcaacaatctggacaattcagaccccaaacatctctttctccctctacagcatcttttgaagagtgtgaataattatgtcaactctttgttttcttataccaaagtgttactgatgaagcactatggcaaaataatgctttcatataaaatactttttcttaaataaatacaaaaatacgtatttgtaaaactaaatgtggataaagttttctttgtcaataatgtaataaatattgagaagggtgtggtttggacaatcatagcttactggtttttcagctaatggcaaaataaaattccatcaaatgtcctgtgcattatcacaatgtgtgtgtctaaacaccttggatgctgttcccctattttgtcctaagttgctcaatggatttcttgagaaatttgtaaccccagtgggtaggacttttctctattcatccacatacgtgtattcaccctgttttactcatttataaggattaatccacagcctctattgagaccatgttcttcccagtttgagactgtcttctcaaaaagagggaaaaaatggtcatcaatactcagttggaaaaaatccattttatacaccaaatcatttctgctggcaaacactaggtagtaggactggaatggagtcagagagaaggaaaactttcactttttagtttatgcattgctactatttgaatgtttacatcaaagcatgtattaggaatccattttaatgaacaattggactagaaattcagctgtgcctgtggctttaatttgaaagcctcctggccagctcactatgtacctagaaactcagttcctgccagtcttggcagacaccattgcatattattaccctgcatgactttgttgtcgaggtcacttgtcgcaagtcacgaccttcactgtcttctctagacggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatggtggcggatcccgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctacccgggctgcaggaattctaccgggtaggggaggcgcttttcccaaggcagtctggagcatgcgctttagcagccccgctggcacttggcgcatcacaagtggcctctggcctcgcacacattccacatccaccggtagcgccaaccggctcccttctttggtggccccttcgcgccaccttctactcctcccctagtcaggaagttcccccccgccccgcagctcgcgtcgtgcaggacgtgacaaatggaagtagcacgtctcactagtctcgtgcagatggacaagcaccgctgagcaatggaagcgggtaggcctttggggcagcggccaatagcagcttggctccttcgctttctgggctcagaggctgggaaggggtgggtccgggggcgggctcaggggcgggctcaggggcggggcgggcgcgaaggtcctccggacccggcattctgcacgcttcaaaagcgcacgtctgccgcgctgttctcctcttcctcatctccgggccttcgacctgcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagattttagtggacatctttattgtttaatagatcatcaatttctgcatactaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 46 - DNA, 9626 bp, Artificial Sequence
  Description of Artificial Sequence Synthetic recombinant DNA vector sequence
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcttgcggccgcgaaggaagatagccaaggcttactgaggctggtggagggagccactgctgggctcaccatggaccgccggatgtggggggcccacgtcttctgcgtgttgagcccgttaccgaccgtgagtagccagctgagaccctctggcctctgccactgggctccagatttgggagccacagggccaactggagcctggcggagaccctactgcccttctcctgctctagagtccctccctacgagcaggtggcaggctgtggcttggagagcccctgacactggggctccctccctcatctccctctgcccagcaatctgtagtgactctgggtagtgctattaacatgtccttccaggggctgagctgagctcaccttcttgttgcttcccccagggcctggctgtgcctctgggggccacagagactctgtctgccacaagacagtgcctcagaattggtgaggcaaagtcaggctcccctcttagtccctgcagtttgctctttctctcagctgtgcacctgtgatgcttccacacgctcctgtggtaagctttctggacccttccttagccctatgactccctgagccactctccagtttggtggtttcctttgaagagggtgtggccagggaaggaatggaacctaaggagttggtggagaccagctgcttctgctccagcacttaggggatccatcatcttctcacgatgctgagggctggagaggcttttggcaaccaaattgtactccatgggctagagcgctggagcacggggcatcaggagagagggcccttcctgaatgggctgcagtttcccagtaactttccaggaggttactgcaagtgacagcagggaccaggctgggagtcacaaggttctgggatggtgagggcctgaagcattcttcctccgctgccttattggagggtgctgcccctcccgagcaccttgctgcacctgctcctgagtgcacaggctttgagcttgaatcctggacgcatgaaactagccgtgtgaactgcaagttcctcagccttcctgagtcttgcttttatctcttttgttaaaagagaaaatatccgctggcccgggtcactgtgcattgagtggaatctgatttgtcaagggctgggcataggggtgcaacacaggaaacttcgactgcccccctgtctgccatctgtgtgcactctacagcaaaagctttatttttagcaggggtacccaagatctggtggtggccattcagcagatggtgctagtaccaaagctagaactcaatcactgtttgccagtgtgggcatcatcccctgcagataccaggaagagcagcaggcatagatgcaaggcctgggggtccagggcgatggagcctcccctcaggggtgggtggggtccacgatgtgtgtgggggggtgactgggagccccaggatgtgtttcgccagtcctaattcagcaggagcatttggataaatcttagaacgatctatccttccactcccaccacttccctccctagaatctctccttacatctggccctatgcccacacatcctgagtcattagtggggcttctctgaatttccctccttctcttagaggtgtgggcactgtctgttcatccttttggcatgaggctgctctcctggcatcctttgccctcagttgcttccccaaattggccctgaaaagtttcccaggcagggttggggcttgccagactgtgcagtaggcagaaggataggcagagaccacagctcttccaacagcccagggtcaatatcaggctgatggtgtgagaaagtcccatacaggcccatctgccctcctgctcttccttccccgctttgtgcgctcagtctgttgggagatgtaggggagacgcaacggggctctgccctgagatggacctgaggttactgagtcctcgggaggaagccaagagctgagtccggggcacccaccaaaataagacctggagcctgggtagtgggtgacatctgcaccctggggccttgcagggccaggcaggagatggcagagctgcgtccaagccatggacccatggggtcagcagggcgggacaggagtgacttctcctgccagggtagcactggtgggggtgaccctccttactgggctggctctccagattttctcctatcccacaaatctgcggcagagctctcgggtctctccacgtggtcacccaccatctattgtccccactctggtggtcaaagtctctactctctgaccccagccctggtcttacatctgctacactatttgctgtgcctgcatttctcccaaagaacactagctgttaattaaacatttgctgacatggatcctgctagacattgagggtgtgtgtgtgtggggtgggggtgggggccatggggagagaaacattagtcctgagctgatacttcccaaatacaccttttcccacttcccttgtccttgcacatgctgtgctttctgttccagatgctcttctccccactgtgtgtgtctggctctaaaaaatgctcccttgtccccaggtccagctcaaatgccacctccccaatccctgtgagagggacattccctcccatggtacctgtcttgcttctttttgttttttttttgtttttctgagatggagtctcactttatcgcccaggctggagtacagtggcacaatctcagctcactgtaacctccgcctcctgggttcaagtgattctcctgcctcagcctcctgagtagctgggattacaggagcgtgtcatcacacccagctaatttttgtatttttgtagagatagtgttttgccacattggtcaggctggtgtttaactcctgacgtcaggtgatccgcctaccttggcctcccaaagtgctgggattacaggcatgagccactgcgcccggcccctgtcctgcttctgatcacagtttgcgcagggtctattaggatccagcccttctcccttccccagcccctcctctcctgtgaaagcctgggggaacagacatctacactggttgacttggatgtgtgttttatacaaaaatgtgtatgctgtatacccagttcacagtgcagaaagtatgttttgtaaacttggcttcccttgtatgtatctgtttgggctctggcctgctgggcgatcagttttggttttccttcggtttggggcggtgggagggagaagtgctagaaattcctgctgttcacttccagtttcctggagaacttgttaaaatacaaagtgctggatccaccctagatctctacatattcctgcttccatcccaatggatctctagctggttcttgaggcacactttcagaagtactgctagaagattcccaaaaaaaccccaacttgtttccagcttccagagatcatttctcatggccactgtgtagcacaagatctgggaggctcctaaaactataatctttcaaggttgctcagccatcaaggttatgatcctttcatctacacatccatgaagccacacatccatccttacacagctctctcactcatccaccatgcacccatccatgcagccagtcatctgtctatccatccatccacctccccagccatatatttgtctgtctgtctgtatactcatccattccactagccttccttccttcttttctgccttcctgccttcctgccttccttccctccttcctttttttatgcagccttcacctcattcatgtgctcactcatccatgtgctcattcattcatccatcttctctctcacttactaagctacccatttgctcatgtgttcctgcctgtgtgcacccctaaaagcagagactgagacaaaggtcagaaatttaaaatggtttccagtggggtaaatccaaagtattggcagagctgcattcctcctggagactgtagagaataatccatttccttgccttttccttgggttcatgccctttctccatcttcagaccagcagtgtggcatctttaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 47 - DNA, 22 bp, Artificial Sequence
  Description of Artificial Sequence Synthetic primer
gagaacccactgcttactggct

SEQ ID NO: 48 - DNA, 21 bp, Artificial Sequence
  Description of Artificial Sequence Synthetic primer
gcattgctgtgcagtctgtaa

SEQ ID NO: 49 - DNA, 7354 bp, Artificial Sequence
  Description of Artificial Sequence Synthetic recombinant DNA vector sequence
cgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagattttagtggacatctttattgtttaatagatcatcaatttctgcatactaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtgcggccgggccatgcaggccagacatgataagatacattgatgagtttggacaaaccacaactagaatgcagtgaaaaaaatgctttatttgtgaaatttgtgatgctattgctttatttgtaaccattataagctgcaataaacaagttgtcgagttacttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgttagcttgatttgacagtggctgggggttgcgccgccgggttttataggaagccacagcggccactcgagccataaaaggcaactttaggaacggcggggggtgattggattcgagtcgtttattcaccggccttgccgcacagtgcagcatttttttaccccctctcccctccttttgcgggggaaaaaaaaaaaaaaaaaaaaaaaaggagaagagaaaaaaagcgagcgagagagaaagcgagattgaggaagaggatgaagagtttggcgatgggtgctggttccgtaggcccagatggacaagaatagcccccgcccttgcggacagtatcccattcagtgactcagatcagtcaagcggccgctctagacggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatggtggcggatcccgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctacccgggctgcaggaattctaccgggtaggggaggcgcttttcccaaggcagtctggagcatgcgctttagcagccccgctggcacttggcgcatcacaagtggcctctggcctcgcacacattccacatccaccggtagcgccaaccggctcccttctttggtggccccttcgcgccaccttctactcctcccctagtcaggaagttcccccccgccccgcagctcgcgtcgtgcaggacgtgacaaatggaagtagcacgtctcactagtctcgtgcagatggacaagcaccgctgagcaatggaagcgggtaggcctttggggcagcggccaatagcagcttggctccttcgctttctgggctcagaggctgggaaggggtgggtccgggggcgggctcaggggcgggctcaggggcggggcgggcgcgaaggtcctccggacccggcattctgcacgcttcaaaagcgcacgtctgccgcgctgttctcctcttcctcatctccgggccttcgacctgcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatg

SEQ ID NO: 50 - DNA, 10514 bp, Artificial Sequence; identical to SEQ ID NO: 45
  Description of Artificial Sequence Synthetic recombinant DNA vector sequence

SEQ ID NO: 51 - DNA, 10247 bp, Artificial Sequence
  Description of Artificial Sequence Synthetic recombinant DNA molecule
ctagaactagtggatccggataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaaccccgggctgcaggaattctaccgggtaggggaggcgcttttcccaaggcagtctggagcatgcgctttagcagccccgctggcacttggcgcatcacaagtggcctctggcctcgcacacattccacatccaccggtagcgccaaccggctcccttctttggtggccccttcgcgccaccttctactcctcccctagtcaggaagttcccccccgccccgcagctcgcgtcgtgcaggacgtgacaaatggaagtagcacgtctcactagtctcgtgcagatggacaagcaccgctgagcaatggaagcgggtaggcctttggggcagcggccaatagcagcttggctccttcgctttctgggctcagaggctgggaaggggtgggtccgggggcgggctcaggggcgggctcaggggcggggcgggcgcgaaggtcctccggacccggcattctgcacgcttcaaaagcgcacgtctgccgcgctgttctcctcttcctcatctccgggccttcgacctgcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgcctgagaaaggaagtgagctgtaaaggctgagctctctctctgacgtatgtagcctctggttagcttcgtcactcactgttcttgactcagcatggcaatctgatgaaatcccagctgtaagtctgcataaattgatgatctattaaacaataaagatgtccactaaaatggaagttttttactgtcatactttgttaagaagggtgagaacagagtacctacattttgaatggaaggattggagctacgggggtgggggtgggggtgggattagataaatgcctgctctttactgaaggctctttactattgctttatgataatgtttcatagttggatatcataatttaaacaagcaaaaccaaattaagggccagctcattcctccactcatgatctatagatctatagatctctcgtgggatcattgtttntctgatccactggaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtgcggccgggccatgcaggccacgacatgataagatacattgatgagtttggacaaaccacaactagaatgcagtgaaaaaaatgctttatttgtgaaatttgtgatgctattgctttatttgtaaccattataagctgcaataaacaagttcttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgttagcttgatttgacagtggctgggggttgcgccgccgggttttataggaagccacagcggccactcgagccataaaaggcaactttaggaacggcggggggtgattggattcgagtcgtttattcaccggccttgccgcacagtgcagcatttttttaccccctctcccctccttttgcgggggaaaaaaaaaaaaaaaaaaaaaaaaggagaagagaaaaaaagcgagcgagagagaaagcgagattgaggaagaggatgaagagtttggcgatgggtgctggttccgtaggcccagatggacaagaatagcccccgcccttgcggacagtatcccattcagtgactcagatcagatcaagcggccgccagtgtgatggatatctgcagaattccagcacactggcggccgttactagtggatcctcaatcagtccatcacctgggtactttttctacccaatgctgaaagtgaatatttgagaaactatgtttctaactggaagaattctgaaactatgttcttctacaacatgtagacgatgtgtcacaatgaaaatgaaagttccttctgaagagctgtaaccctacatagcctaggatgccatttatactttctctagcccagaggctctgtatttctgctctcaggctacattactacataaccatcaaaccaactgcccgaaattgctgagcaattgatcataacatacagacctgattcagatatataaattacatttttatttttatcgtaccaaaacatacaggtgaaaactttcaaattcaggcaactgacatgccaggtgcctacctatctgtgaagaccagagcactgtaagacacggaatagctggttgaaaagtgaaaaatccagtgagttaggttgtttgattatacagaatcatagaattttcaaagtgaaaggacatggtgatcactgatttcagtgactatccaacaacattagcaagagcccctagggttccctaccagttccttcagaagcctccttcaaagctgtgagtgcatggccaaaggggaagctacattcatcaaccagactaatcagagaccagggagcagaaataaggttaacacggaatggagtacatttgctttgggggacgttcatgttttcttgcgcatactgaaaacagctgggtgctgattttggtataacactcagagcctcttttgataaaaaggcattctatatgaatgaaaaccctgttggcttcacattggtcaaagcaatggattttaaatagtgactcaacttcctagaaaactttgtatttagaaatgttttcttcatacagctaacaggcaaaactaatcaaaaccttgttgttagtctgatgtttattttctgtgcagcaaacaaactcccataaaaacctctgaggttttcttagttatgtttgcaattttttaagtgttgaactgcctaagtctctgatgtcatgacacttaattcatatattttcttgcttttgctagaactgaacaaacactccctatcaagattgaccctctctagaaataaaataaattgtcatatgaagtcaaaatatgacctatcaagtttatgatagctttctttgacatttatgggaggagaaaattataccatccatactacataggttggggtttaaataatatttgcctataaacaataagcaattgcagattgattttaagtaaacgtatgcaatgcctgtaagcaacaggaaatatattttacatcaatgtatgcactgcctgtaagtaacaggaattatattttacatgaatgtgtgaagtgataattttttaataggcaaaaattagtgttaaaagcggaactggcatgctttcctaggctgtctcagtgataatgacctcaaagtcaatgatttcttcatgaagatgaaataagtttattgcaacttgattgcatcacctaaaatacaacccttgacttggaagcttgcccttatgttttatgaggtctgaaataaaaaggaaactgatggaactgaaccagcaaattattttgcaatccatgactacagttgtatacagcagatgtagtcaattgttgaggaaagttggaccaaatttccatcctgttcagtactgctgatagctctattcaaaatacattcttgcctttagaggaactgacccattagtaatcatacagatagaattttgtgaactatttttaaagggtaggttattaatcaatgagtgactctcaaatatcttgatggtttccactgtgaaaaccaagtatctgcatgatagagcaaaaatttaattttctcagatgctttagagtacatatagatgaagactgccttcttactagatcagttttgagagagatgaaaacaaaagaaagaaaaaataggatagctgatgaaatgaaacttcaaatttaatttgatcatgttggcattttttattccttgagtgcattgaaaataactgattaatactagacacatataatactatcaagttacacctaaaatttattttgtcagccaggcaaccaatcagaaccaaatagtgaccaagaattattttttcaaacacgtttgtcttccaggaccttactgagttagttccacagatcatgaaaacatgggtcttttctcaggtcttctacatcatttcccttaagttgacacccaaagtcataagcttgactttagacacaggtttcaaaagtagaaagttttattctcttatgtttctatggctttgaagttttgcagaactataaaaataatttgttttaccctttccacaactcatggacgttccttgaagtgaaggaaacgaatactcgtctttgcataataaacagcaaaaaaaaaaaaaaaaaaaaaattaaatgaatctaaattctcatatgccatttaaagtttcagacccagacatcttcaggattttcttacatttaaagcagaatgacactactgatacacaaaagtaaattttaattctgtttttaatgcatgaggcaatataaaaattaatcatgatgatcattatatttgttctaacactaaagtttttcccataatcccccagatttttcaagtctcttttctcctcatggcagatgcccaaccacagagagaaaggttctataataaaactttaaaaacactgacctaaaagagaaatgttttatcgccatgcaatattctaaattctggcatttggtggaaaaactgtacacatttttggcaatggctttagtgtccactggtctggcagctataatgagaataataaagtatatggtgatgactgtttacttaccattatgagacatccccactgcggtacccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatggtggcaagcttaagtttaaacg

SEQ ID NO: 52 - DNA, 10514 bp, Artificial Sequence; identical to SEQ ID NO: 45
  Description of Artificial Sequence Synthetic recombinant DNA vector sequence

SEQ ID NO: 53 - DNA, 168 bp, Homo sapiens
cggaggacagtactccgctcggaggacagtactccgctcggaggacagtactccgctcgaggacagtactccgctcggaggacagtactccgatccgtcgactctagtccgctcggaggacagtactccgctcggaggacagtactccgctcggaggacagtactccg

SEQ ID NO: 54 - DNA, 22 bp, Artificial Sequence
  Description of Artificial Sequence Synthetic primer
gagaacccactgcttactggct

SEQ ID NO: 55 - DNA, 20 bp, Artificial Sequence
  Description of Artificial Sequence Synthetic primer
caagatcgccctcgcctttg

SEQ ID NO: 56 - DNA, 33 bp, Artificial Sequence
  Description of Artificial Sequence Synthetic primer
atatggtaccgcagtggggatgtctcataatgg

SEQ ID NO: 57 - DNA, 30 bp, Artificial Sequence
  Description of Artificial Sequence Synthetic primer
aattggatcctcaatcagtccatcacctgg

SEQ ID NO: 58 - DNA, 30 bp, Artificial Sequence
  Description of Artificial Sequence Synthetic primer
cgtctagagagtgtgtcctgtcagaagaac

SEQ ID NO: 59 - DNA, 31 bp, Artificial Sequence
  Description of Artificial Sequence Synthetic primer
atgcggccgcactcctgacctcaggtgatcc

SEQ ID NO: 60 - DNA, 29 bp, Artificial Sequence
  Description of Artificial Sequence Synthetic primer
cgtctagagaagacagtgaaggtcgtgac

SEQ ID NO: 61 - DNA, 29 bp, Artificial Sequence
  Description of Artificial Sequence Synthetic primer
gcgcggccgcgtctaacctaggagcccac

SEQ ID NO: 62 - DNA, 28 bp, Artificial Sequence
  Description of Artificial Sequence Synthetic primer
gctctagaggacgaatgccaagatctga

SEQ ID NO: 63 - DNA, 29 bp, Artificial Sequence
  Description of Artificial Sequence Synthetic primer
caagcggccgccagtgtgatggatatctg

SEQ ID NO: 64 - DNA, 5218 bp, Artificial Sequence
  Description of Artificial Sequence Synthetic recombinant DNA sequence
gccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatccgatgttttctgagttacttttgtatccccaccccccctcgaggatcgatccgctcggaggacagtactccgctcggaggacagtactccgctcggaggacagtactccgctcgaggacagtactccgctcggaggacagtactccgatccgtcgactctagtccgctcggaggacagtactccgctcggaggacagtactccgctcggaggacagtactccgctcgaggacagtactcgattcgaggacattttgacacccccataatatttttccagaattaacagtataaattgcatctcttgttcaagagttccctatcactctctttaatcactactcacagtaacctcaactcctgcccaagcttggtaccaccatggacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaatctagagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtc

SEQ ID NO: 65 - DNA, 28 bp, Artificial Sequence
  Description of Artificial Sequence Synthetic primer
ggggtaccaaagaaggtaggctgagggg

SEQ ID NO: 66 - DNA, 32 bp, Artificial Sequence
  Description of Artificial Sequence Synthetic primer
cgggatccgtacaagacagttagctagttggc

SEQ ID NO: 67 - DNA, 34 bp, Artificial Sequence
  Description of Artificial Sequence Synthetic primer
ggggtaccattcagcaggccactacaggactctc

SEQ ID NO: 68 - DNA, 32 bp, Artificial Sequence
  Description of Artificial Sequence Synthetic primer
gatcgcggccgctgtgctcgacattggtggcc

SEQ ID NO: 69 - DNA, 31 bp, Artificial Sequence
  Description of Artificial Sequence Synthetic primer
ggggtacctttgtggtgcttaaaaaatgagc

SEQ ID NO: 70 - DNA, 33 bp, Artificial Sequence
  Description of Artificial Sequence Synthetic primer
gatcgcggccgcctcatgaacaatgaaatctcc

SEQ ID NO: 71 - DNA, 2379 bp, Artificial Sequence
  Description of Artificial Sequence Synthetic Nurr1 targeting sequence
cgtacaagacagttagctagttggcaaaaccaaggagaattctgtgacaagggaaactcaggacaaatcctgctaggcagttctggaggggaagcgccctgcgcctgcagtactgacctgtgaccatagccagggcagcaatgcaggagaaggcagaaatgtcgatgttcatattctgcaagttggaggagaattcaacaatggaatcaatccattccccaaagccacgaacgcattgcaacctgtgcaagaccaccccattgcaaaagatgagtttaccctccactgggttggacctgcaattaataccaaagagagagaggggagaaaaagagagagagaaaagctaaacaactaattacttgaagagcaataaatgagggatttaagagtcacctaattactgaagagttaattaaaatgtagatccagtggaccttgaaagggtttaatttcataacaatcaagaacgccccctatcccacctccattccattcatccaaggcttctgggctcgcttcttctcgtctttttttgttttctttctttttcttcctttctccgacttccatttcctattctgtctttttctctaccccaccctctggtttcccttcctccctttcttttcctttcttgatttctctcacagcctccctggattgtctcctccctcccttattacctgtatgctaatcgaaggacaaacagttctaagaaagctgattcaaaaagcaggtcttggtcggctttgggcaggtctgcgaagccagggatcttctctgcccagccccggatgatctccatggagccagtcaggagatcatagaattgctggatatgctgggtgtcatctccactcatttgatagtcagggttcgcctggaactggaatttcattttaaaaagcacttaatgaggttctctaaaatatataacccgtgaaattgctaaccccgtttctaataggggagccaggtttttataacaattaaacctctctctgaccagtaaggaaattagatgttccggggcaattaattcaattagggatggtgctacgaggtcgctgctttaaatatgtaaattgtcatttccatacagactaaatacagtgccatccagccctgggaaacgttcactcttatctccacaaaacaattgacatgttagtattcatgctagtcccagagtgggcctcgagctgagacgcccttttgcaaatcttaataaaattgcagtccctaccaggtttgctcaacatacttcctccaattaaagcccgcatatttttcacagctggagtaaaaggatctgaccacttgatcccccaccccactggcatctttacaccctctccttcccttccctgacccccagacagcccccacatccttccccagcacagtgccaggaaggatgaggattaaagcatctatctctatggaggactgggaataaagtagattgggaagggggagagtttgcacagcccctggatttcatggtagaggcactggtgtatatggaagaggaggggcgatggggtagtggggtagtgggaattcacacagcttcaaaccctgtggcgtgtccctctcctccttcctccatcctgtcctcatgtctatgcccccagctacatggatttgtctgaagatctgaatcttgaggccctggccagagctgcgaggcatatacagccttgcttgcctttctttacccccgttgaatctgagagttaatgacggatgtggggaggggtcctgcccatctgtcggtttgtccacatgatatcccccccgccagcttcttaccctggaatagtccaggctggtcatagccgggttggagtcgacatgggccctgacgagggcactgatcagactcaccgggggcgaagggggagagggctcctgtgggctcttcggtttcgagggcaaacgacctctccggccttttaaactgtctgtgcgaaccactgcaaaggaagagccctgttagcgccgcttttccgagcccaggcccagctgctgcctcggtccctccccggggaaggccgcagccgcggggcaccaggctgagcggctgagggccccagtgcttgtaaagccttcactgactagaagcattaaaaaatgcggggttattttatgtcttcctccaaatgggtcgtatagttaaaggagagaagggcctggcggctttctctagggaaggccgggcaagcaggcagctgcagggtcctggaggccatactgagggggagtcggagatccccagcaccgggaagtggaacgtgatgctggagtatgagcagtggtttcctaaaggcgcaaactggagggtcggcagctcccctcagcctaccttctttg

SEQ ID NO: 72 - DNA, 8426 bp, Artificial Sequence
  Description of Artificial Sequence Synthetic pCDGal4-DBD-Nurr1 sequence
tctctggctaactagagaacccactgcttactggcttatcgaaattaatacgactcactatagggagacccaagctggctagcgtttaaacttaagcttgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccgggtaccaaagaaggtaggctgaggggagctgccgaccctccagtttgcgcctttaggaaaccactgctcatactccagcatcacgttccacttcccggtgctggggatctccgactccccctcagtatggcctccaggaccctgcagctgcctgcttgcccggccttccctagagaaagccgccaggcccttctctcctttaactatacgacccatttggaggaagacataaaataaccccgcattttttaatgcttctagtcagtgaaggctttacaagcactggggccctcagccgctcagcctggtgccccgcggctgcggccttccccggggagggaccgaggcagcagctgggcctgggctcggaaaagcggcgctaacagggctcttcctttgcagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccagggtaagaagctggcgggggggatatcatgtggacaaaccgacagatgggcaggacccctccccacatccgtcattaactctcagattcaacgggggtaaagaaaggcaagcaaggctgtatatgcctcgcagctctggccagggcctcaagattcagatcttcagacaaatccatgtagctgggggcatagacatgaggacaggatggaggaaggaggagagggacacgccacagggtttgaagctgtgtgaattcccactaccccactaccccatcgcccctcctcttccatatacaccagtgcctctaccatgaaatccaggggctgtgcaaactctcccccttcccaatctactttattcccagtcctccatagagatagatgctttaatcctcatccttcctggcactgtgctggggaaggatgtgggggctgtctgggggtcagggaagggaaggagagggtgtaaagatgccagtggggtgggggatcaagtggtcagatccttttactccagctgtgaaaaatatgcgggctttaattggaggaagtatgttgagcaaacctggtagggactgcaattttattaagatttgcaaaagggcgtctcagctcgaggcccactctgggactagcatgaatactaacatgtcaattgttttgtggagataagagtgaacgtttcccagggctggatggcactgtatttagtctgtatggaaatgacaatttacatatttaaagcagcgacctcgtagcaccatccctaattgaattaattgccccggaacatctaatttccttactggtcagagagaggtttaattgttataaaaacctggctcccctattagaaacggggttagcaatttcacgggttatatattttagagaacctcattaagtgctttttaaaatgaaattccagttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtaataagggagggaggagacaatccagggaggctgtgagagaaatcaagaaaggaaaagaaagggaggaagggaaaccagagggtggggtagagaaaaagacagaataggaaatggaagtcggagaaaggaagaaaaagaaagaaaacaaaaaaagacgagaagaagcgagcccagaagccttggatgaatggaatggaggtgggatagggggcgttcttgattgttatgaaattaaaccctttcaaggtccactggatctacattttaattaactcttcagtaattaggtgactcttaaatccctcatttattgctcttcaagtaattagttgtttagcttttctctctctctttttctcccctctctctctttggtattaattgcaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacaggtcagtactgcaggcgcagggcgcttcccctccagaactgcctagcaggatttgtcctgagtttcccttgtcacagaattctccttggttttgccaactagctaactgtcttgtacggatccactagtaacggccgccagtgtgctggaattctgcagatatccatcacactggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagc

SEQ ID NO: 73 - DNA, 9701 bp, Artificial Sequence
  Description of Artificial Sequence Synthetic pKI-Gal4-DBD-Nurr1 sequence
tgcacgcttcaaaagcgcacgtctgccgcgctgttctcctcttcctcatctccgggccttcgacctgcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgcctgagaaaggaagtgagctgtaaaggctgagctctctctctgacgtatgtagcctctggttagcttcgtcactcactgttcttgactcagcatggcaatctgatgaaatcccagctgtaagtctgcataaattgatgatctattaaacaataaagatgtccactaaaatggaagttttttactgtcatactttgttaagaagggtgagaacagagtacctacattttgaatggaaggattggagctacgggggtgggggtgggggtgggattagataaatgcctgctctttactgaaggctctttactattgctttatgataatgtttcatagttggatatcataatttaaacaagcaaaaccaaattaagggccagctcattcctccactcatgatctatagatctatagatctctcgtgggatcattgtttntctgatccactggaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtgcggccgggccatgcaggccacgacatgataagatacattgatgagtttggacaaaccacaactagaatgcagtgaaaaaaatgctttatttgtgaaatttgtgatgctattgctttatttgtaaccattataagctgcaataaacaagttcttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgttagcttgatttgacagtggctgggggttgcgccgccgggttttataggaagccacagcggccactcgagccataaaaggcaactttaggaacggcggggggtgattggattcgagtcgtttattcaccggccttgccgcacagtgcagcatttttttaccccctctcccctccttttgcgggggaaaaaaaaaaaaaaaaaaaaaaaaggagaagagaaaaaaagcgagcgagagagaaagcgagattgaggaagaggatgaagagtttggcgatgggtgctggttccgtaggcccagatggacaagaatagcccccgcccttgcggacagtatcccattcagtgactcagatcagatcaagcggccgccagtgtgatggatatctgcagaattccagcacactggcggccgttactagtggatccgtacaagacagttagctagttggcaaaaccaaggagaattctgtgacaagggaaactcaggacaaatcctgctaggcagttctggaggggaagcgccctgcgcctgcagtactgacctgtgaccatagccagggcagcaatgcaggagaaggcagaaatgtcgatgttcatattctgcaagttggaggagaattcaacaatggaatcaatccattccccaaagccacgaacgcattgcaacctgtgcaagaccaccccattgcaaaagatgagtttaccctccactgggttggacctgcaattaataccaaagagagagaggggagaaaaagagagagagaaaagctaaacaactaattacttgaagagcaataaatgagggatttaagagtcacctaattactgaagagttaattaaaatgtagatccagtggaccttgaaagggtttaatttcataacaatcaagaacgccccctatcccacctccattccattcatccaaggcttctgggctcgcttcttctcgtctttttttgttttctttctttttcttcctttctccgacttccatttcctattctgtctttttctctaccccaccctctggtttcccttcctccctttcttttcctttcttgatttctctcacagcctccctggattgtctcctccctcccttattacctgtatgctaatcgaaggacaaacagttctaagaaagctgattcaaaaagcaggtcttggtcggctttgggcaggtctgcgaagccagggatcttctctgcccagccccggatgatctccatggagccagtcaggagatcatagaattgctggatatgctgggtgtcatctccactcatttgatagtcagggttcgcctggaactggaatttcattttaaaaagcacttaatgaggttctctaaaatatataacccgtgaaattgctaaccccgtttctaataggggagccaggtttttataacaattaaacctctctctgaccagtaaggaaattagatgttccggggcaattaattcaattagggatggtgctacgaggtcgctgctttaaatatgtaaattgtcatttccatacagactaaatacagtgccatccagccctgggaaacgttcactcttatctccacaaaacaattgacatgttagtattcatgctagtcccagagtgggcctcgagctgagacgcccttttgcaaatcttaataaaattgcagtccctaccaggtttgctcaacatacttcctccaattaaagcccgcatatttttcacagctggagtaaaaggatctgaccacttgatcccccaccccactggcatctttacaccctctccttcccttccctgacccccagacagcccccacatccttccccagcacagtgccaggaaggatgaggattaaagcatctatctctatggaggactgggaataaagtagattgggaagggggagagtttgcacagcccctggatttcatggtagaggcactggtgtatatggaagaggaggggcgatggggtagtggggtagtgggaattcacacagcttcaaaccctgtggcgtgtccctctcctccttcctccatcctgtcctcatgtctatgcccccagctacatggatttgtctgaagatctgaatcttgaggccctggccagagctgcgaggcatatacagccttgcttgcctttctttacccccgttgaatctgagagttaatgacggatgtggggaggggtcctgcccatctgtcggtttgtccacatgatatcccccccgccagcttcttaccctggaatagtccaggctggtcatagccgggttggagtcgacatgggccctgacgagggcactgatcagactcaccgggggcgaagggggagagggctcctgtgggctcttcggtttcgagggcaaacgacctctccggccttttaaactgtctgtgcgaaccactgcaaaggaagagccctgttagcgccgcttttccgagcccaggcccagctgctgcctcggtccctccccggggaaggccgcagccgcggggcaccaggctgagcggctgagggccccagtgcttgtaaagccttcactgactagaagcattaaaaaatgcggggttattttatgtcttcctccaaatgggtcgtatagttaaaggagagaagggcctggcggctttctctagggaaggccgggcaagcaggcagctgcagggtcctggaggccatactgagggggagtcggagatccccagcaccgggaagtggaacgtgatgctggagtatgagcagtggtttcctaaaggcgcaaactggagggtcggcagctcccctcagcctaccttctttggtacccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatggtggcaagcttaagtttaaacgctagaactagtggatccggataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaaccccgggctgcaggaattctaccgggtaggggaggcgcttttcccaaggcagtctggagcatgcgctttagcagccccgctggcacttggcgcatcacaagtggcctctggcctcgcacacattccacatccaccggtagcgccaaccggctcccttctttggtggccccttcgcgccaccttctactcctcccctagtcaggaagttcccccccgccccgcagctcgcgtcgtgcaggacgtgacaaatggaagtagcacgtctcactagtctcgtgcagatggacaagcaccgctgagcaatggaagcgggtaggcctttggggcagcggccaatagcagcttggctccttcgctttctgggctcagaggctgggaaggggtgggtccgggggcgggctcaggggcgggctcaggggcggggcgggcgcgaaggtcctccggacccggcattc

SEQ ID NO: 74 - DNA, 2379 bp, Artificial Sequence; identical to SEQ ID NO: 71
  Description of Artificial Sequence Synthetic nucleotide sequence for GR region of homology

SEQ ID NO: 75 - DNA, 9597 bp, Artificial Sequence
  Description of Artificial Sequence Synthetic pCDGal4-DBD-GR sequence
taacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccgggtaccattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtaagatgcaaaacataaaagagcaactatataaacctttgtgttttcttcagcaaaaacactttggcttttatatcatcgtgagcccatggcttatcttgtttctcttagttctggggactatgaaggggagagtcaggtgaatacaggtgatagggagtttataataaaacatttacattactccctgcttttcaaatcattatgcacaggatggtaatttcacataggatgatgtaatatcagaattcaagttacaagactcactcaaaactccttttacactgaagtttggggaaagaaaatgtttttagttaattccatttgttttccttcattgtgccacttttaaaaatcaggttgtttgtaagattggtaaacatcaagtatgttgattgtcaaaatttgtactaaagtagaatgattttaacccttcactaaatgaaatgctacacattgaatgtaattttaaagataattttaaataaaagttaccctattggaatttggtgtggaatggcagaggtcaatgttagtgtcagctctgactttaaagacagggaattgacaagcctgtgttcacgcaaatagttagggagagagcaagaaagtaacctgacctcctgtcatccttgttttattaagggggaaagaggtgtgaatagcagggcaaatgttttgcttaactcattgattaatacctcaagccaagattcttttctgttttttaaaatcaatacataatagttgtacatatttactgtacatatttatatttagggggtacatgtaataatttaataaaagcatacaacgtgtaaggatcaaatcagagtaactgggatatccatcacctcaaacatttgtttggggaacattccaaatcttctcttttagctattttgaaatataaagtaaattattgttaactatagtcatcctgttgtgctactgaacactaaaacttatttcttctaactgtatttttgcacccgtcaaccattcccgcttcatccccatcaccactatctttcccggtcactggtaaccgccaagccaagaattttggctattttactatttagttcatgtttacttaagcagacagaggtgacaaaactggctttttttttttttttttacattaaaagctattaaaaagcacctagggggctgggtgcgatggctcacgcctgtaatcccagcactttgggaagcccaggtgggtggatcagttgaggtcaggagttcgagaccagcctggccagcatagcaaaaccccatctctactaaaattacaaaaattagccgggcatggtggtatgaatctgtattcctagctacttgggaggctggcactgagaatcacttgaacccgggaggcggaggttgcagtgagccgagatggcaccattgcactccagcctgggtgacagagcaagactttgtctcaattaaaaaaaaaaaaaaaaaaaaaaacacaagagggtttgtgagtcttaaagtgtcagatgacagaagaaaactgtgtctacctagtatttaatttccattttctgttaggggtgcccttgttttgacagggctaattgatctcattgctccttggcaattcccacagagatgatcttctgaagagtgttgcctcatacctttatttctcttaattcaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagtaagttgtatgtgtgtcattttccctgtattcatagggtatctttaaccagctgatgttttcctgattgactgctattgtgataattcaggactgaaacaatcctactaggtatctaggatctaggcaaactggaaatagagttatgagtgcttggggcaggacaagtgtaatgtaaagcaaatgtacatgtggcattattactgtcccaggacatgtttgaggatatttaacagcatatctgaggttagtaaagtctgtcgcaagcaacaaggaatcttactgtgatatcatttacataaccctattccagaaagaaaaaggagcatggtaaaactcatgtggattcagtggggacaattgtagatgaggatatctaggctgatggggtgggacatatggacccagacacaagaggtatctctttgcatggcaaggctcacccagtgtctgtggtttaagaatatgggaacaaatttgttttgtttaactgagagaagaccaagcctttaagattttataaatcagctattctcttatcctctaagcttattcctgtgtctgcgaaatacttcaggtgtccatttccccttacctcattgcagttgtttcctcactcgttttctccctccagtgtaacgttcatcatgttggctaatgtttgcttcctcaagcacagtctgactgcatcacatatctccccagtacacagattgtcttcagtatcttcccactgaccctccagtacatattctgcatgatttcagactttccagaatctgacctcacttcctctcccattgttttccttcacacactcttcattcccatccatcctttccagcatactcttagactcttggtgttcacatcaccagatacacagcagagaagtcacatcctagttactctcactttctaccttgtattactacttttcgtacccctagcttattgctattagtacaatgtaaacagggagttcacacacacatacccctggtctaagaagaataaaaaatgaaggagatttctgtttgtatagaaaacagaagtcaccttgacttttattgccaaaaagaggactgttcaaactactgcatcacaatgtaacaagattaggtagttggatccaattttaaattaactggtaaatatatttagtttctggggaaactgaagacattattactcatcataatcctaccatgctgtttaaaaaataccatgttggcagtatttgttttttagtcactttctaatatgtaatttgaaggcatttaagtggaattaaaagcataaacagatttgtatgaaacaccaacttatcctggtttataaaactaacctaatttagggtttttattattagggcattcagatttagctttaagcagtcacagcaaaatctaatcatgccacatacattccttacataaagtgggatttataattttttttcctcaacagatttacattagtttcattttcattaagggatatgtacttcctattcttgtgttctcatgctgctgcctgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgt

SEQ ID NO: 76 - DNA, 10872 bp, Artificial Sequence
  Description of Artificial Sequence Synthetic pKI-Gal4-DBD-GR sequence
cagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtgcggccgggccatgcaggccacgacatgataagatacattgatgagtttggacaaaccacaactagaatgcagtgaaaaaaatgctttatttgtgaaatttgtgatgctattgctttatttgtaaccattataagctgcaataaacaagttcttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgttagcttgatttgacagtggctgggggttgcgccgccgggttttataggaagccacagcggccactcgagccataaaaggcaactttaggaacggcggggggtgattggattcgagtcgtttattcaccggccttgccgcacagtgcagcatttttttaccccctctcccctccttttgcgggggaaaaaaaaaaaaaaaaaaaaaaaaggagaagagaaaaaaagcgagcgagagagaaagcgagattgaggaagaggatgaagagtttggcgatgggtgctggttccgtaggcccagatggacaagaatagcccccgcccttgcggacagtatcccattcagtgactcagatcagatcaagcggccgcaggcagcagcatgagaacacaagaataggaagtacatatcccttaatgaaaatgaaactaatgtaaatctgttgaggaaaaaaaattataaatcccactttatgtaaggaatgtatgtggcatgattagattttgctgtgactgcttaaagctaaatctgaatgccctaataataaaaaccctaaattaggttagttttataaaccaggataagttggtgtttcatacaaatctgtttatgcttttaattccacttaaatgccttcaaattacatattagaaagtgactaaaaaacaaatactgccaacatggtattttttaaacagcatggtaggattatgatgagtaataatgtcttcagtttccccagaaactaaatatatttaccagttaatttaaaattggatccaactacctaatcttgttacattgtgatgcagtagtttgaacagtcctctttttggcaataaaagtcaaggtgacttctgttttctatacaaacagaaatctccttcattttttattcttcttagaccaggggtatgtgtgtgtgaactccctgtttacattgtactaatagcaataagctaggggtacgaaaagtagtaatacaaggtagaaagtgagagtaactaggatgtgacttctctgctgtgtatctggtgatgtgaacaccaagagtctaagagtatgctggaaaggatggatgggaatgaagagtgtgtgaaggaaaacaatgggagaggaagtgaggtcagattctggaaagtctgaaatcatgcagaatatgtactggagggtcagtgggaagatactgaagacaatctgtgtactggggagatatgtgatgcagtcagactgtgcttgaggaagcaaacattagccaacatgatgaacgttacactggagggagaaaacgagtgaggaaacaactgcaatgaggtaaggggaaatggacacctgaagtatttcgcagacacaggaataagcttagaggataagagaatagctgatttataaaatcttaaaggcttggtcttctctcagttaaacaaaacaaatttgttcccatattcttaaaccacagacactgggtgagccttgccatgcaaagagatacctcttgtgtctgggtccatatgtcccaccccatcagcctagatatcctcatctacaattgtccccactgaatccacatgagttttaccatgctcctttttctttctggaatagggttatgtaaatgatatcacagtaagattccttgttgcttgcgacagactttactaacctcagatatgctgttaaatatcctcaaacatgtcctgggacagtaataatgccacatgtacatttgctttacattacacttgtcctgccccaagcactcataactctatttccagtttgcctagatcctagatacctagtaggattgtttcagtcctgaattatcacaatagcagtcaatcaggaaaacatcagctggttaaagataccctatgaatacagggaaaatgacacacatacaacttactcattaataatcagatcaggagcaaaacacagcaggtttgcacttgattgtctatatgatctccaccccagagcaaatgccataagaaacatccaggagtactgcagtagggtcatttggtcatccaggtgtaagttcctgaaacctgaattaagagaaataaaggtatgaggcaacactcttcagaagatcatctctgtgggaattgccaaggagcaatgagatcaattagccctgtcaaaacaagggcacccctaacagaaaatggaaattaaatactaggtagacacagttttcttctgtcatctgacactttaagactcacaaaccctcttgtgtttttttttttttttttttttttaattgagacaaagtcttgctctgtcacccaggctggagtgcaatggtgccatctcggctcactgcaacctccgcctcccgggttcaagtgattctcagtgccagcctcccaagtagctaggaatacagattcataccaccatgcccggctaatttttgtaattttagtagagatggggttttgctatgctggccaggctggtctcgaactcctgacctcaactgatccacccacctgggcttcccaaagtgctgggattacaggcgtgagccatcgcacccagccccctaggtgctttttaatagcttttaatgtaaaaaaaaaaaaaaaaagccagttttgtcacctctgtctgcttaagtaaacatgaactaaatagtaaaatagccaaaattcttggcttggcggttaccagtgaccgggaaagatagtggtgatggggatgaagcgggaatggttgacgggtgcaaaaatacagttagaagaaataagttttagtgttcagtagcacaacaggatgactatagttaacaataatttactttatatttcaaaatagctaaaagagaagatttggaatgttccccaaacaaatgtttgaggtgatggatatcccagttactctgatttgatccttacacgttgtatgcttttattaaattattacatgtaccccctaaatataaatatgtacagtaaatatgtacaactattatgtattgattttaaaaaacagaaaagaatcttggcttgaggtattaatcaatgagttaagcaaaacatttgccctgctattcacacctctttcccccttaataaaacaaggatgacaggaggtcaggttactttcttgctctctccctaactatttgcgtgaacacaggcttgtcaattccctgtctttaaagtcagagctgacactaacattgacctctgccattccacaccaaattccaatagggtaacttttatttaaaattatctttaaaattacattcaatgtgtagcatttcatttagtgaagggttaaaatcattctactttagtacaaattttgacaatcaacatacttgatgtttaccaatcttacaaacaacctgatttttaaaagtggcacaatgaaggaaaacaaatggaattaactaaaaacattttctttccccaaacttcagtgtaaaaggagttttgagtgagtcttgtaacttgaattctgatattacatcatcctatgtgaaattaccatcctgtgcataatgatttgaaaagcagggagtaatgtaaatgttttattataaactccctatcacctgtattcacctgactctccccttcatagtccccagaactaagagaaacaagataagccatgggctcacgatgatataaaagccaaagtgtttttgctgaagaaaacacaaaggtttatatagttgctcttttatgttttgcatcttacctggtattgcctttgcccatttcactgctgcaatcacttgccgccctcctaacatgttgagcgtagtcatgatcctccaagttgagtctggaacagagctatcatatcctgcatataacacttcaggttcaataacctccaacagtgacaccagggtaggggtgagttgtggtaacgttgcaggaactattgttttgttaccaggattttcagaggtttcttgtgagactcctgtagtggcctgctgaatggtacccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatggtggcaagcttaagtttaaacgctagaactagtggatccggataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaaccccgggctgcaggaattctaccgggtaggggaggcgcttttcccaaggcagtctggagcatgcgctttagcagccccgctggcacttggcgcatcacaagtggcctctggcctcgcacacattccacatccaccggtagcgccaaccggctcccttctttggtggccccttcgcgccaccttctactcctcccctagtcaggaagttcccccccgccccgcagctcgcgtcgtgcaggacgtgacaaatggaagtagcacgtctcactagtctcgtgcagatggacaagcaccgctgagcaatggaagcgggtaggcctttggggcagcggccaatagcagcttggctccttcgctttctgggctcagaggctgggaaggggtgggtccgggggcgggctcaggggcgggctcaggggcggggcgggcgcgaaggtcctccggacccggcattctgcacgcttcaaaagcgcacgtctgccgcgctgttctcctcttcctcatctccgggccttcgacctgcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgcctgagaaaggaagtgagctgtaaaggctgagctctctctctgacgtatgtagcctctggttagcttcgtcactcactgttcttgactcagcatggcaatctgatgaaatcccagctgtaagtctgcataaattgatgatctattaaacaataaagatgtccactaaaatggaagttttttactgtcatactttgttaagaagggtgagaacagagtacctacattttgaatggaaggattggagctacgggggtgggggtgggggtgggattagataaatgcctgctctttactgaaggctctttactattgctttatgataatgtttcatagttggatatcataatttaaacaagcaaaaccaaattaagggccagctcattcctccactcatgatctatagatctatagatctctcgtgggatcattgtttntctgatccactggaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatc

SEQ ID NO: 77 - DNA, 3250 bp, Artificial Sequence
  Description of Artificial Sequence Synthetic nucleotide sequence for MR region of homology sequence
tttgtggtgcttaaaaaatgagcatgtggtgcctcctatggtccaggttaataccagactttgataaagttctatggtagaacatgtttccctggcacctgtcctccctgcctgacagactcaagctcttcttttgttctttattttatttatttattttttaagatggattctcattctgtcgcccaggctggagttcagtgacatgatattggctcactgcatcccaggtttcaagcgattctcctgcctcaggctcacaagtagctggggttacaggtgcctgccaggacgcccaactaaattttgtatttttagtagagacagggtttcatgatggtggcaaggctggtctcaaattcctgacctcaagtgatctgcttgcctcagcctcccaaagtctgggattacaggcatgggccactgcaccaggccttcttttgttttttatcacctcccccatctccaggggcatcatgttatttttctgcctgtggagtttcactgggcaagctcctcttcaaagaatcagtcagtggagctttccccaacttttacagctgctgtctgaccacaggtaatctaagaaactgctttagattaatgaactgggacaatgtaatggcaatttctaagtgatgtttctgtagattaagaggggccttattaattaaataccatgaaacaaaaaaaaatgactaaaataggaattccagggacaatttaaagggtgctgggggccaggtgattattttttagacagaagagtcattccaggtctcttacaacttacttgctaggccttgccttacttggtctacacttagattaaccctcgttatgagccgctaataatgtgctcaacttgtgatttcatgaacagaatgtattttaccttattaatatttttggatgaaattttccattttgatcaacataagaagtgtatgtcattttttttatttttaatttcttttttttttttgagacagagtctcactctgttgctcaggctggagtatagtggtgggatctcggctcactgcagcctctgccgcccaggttcaagcgattctcctgcctcagcctcctgagtagctgggattacaggcacctgccaccaagcaggactaatttttgtgtttttagtagaaacggggtttcaccatcttggctaggctggtcttgaactcctgacctcgtgatccatctgcctcagtaagccacctgccttggcctcccaaagtgctgggattacaggcgtgagccactgcgcccagccgtatctgtcattttaacaataaaatccttaaccctgcattctcggaagaaatgagcacattaaccatatttttcctatacacttgatcaatttgttatcaatttaaaaaatctgatatcaggatttcatagaacgcaaactcctcctgcatggttggagatggcgaagtcagttgcccagacttgttaagttcaggatgcagcctgtgaaaggagaggcaatcctacctggaagtacctttgcccacttcacgacttggatcatctgtttgcctgctaagcggttgagcgtggagagcagattttcggctgtatctggttttgagctgtcatagcctgcatatacaatttcaggttcaatgttttcaaggaccataacgggggaaggtgtgagcgctcgtgagattgtggagagctgaggaaccagtgctgtgttgaccgagggttcttttgcaggagcgatgtacgttgtcccttcctctgggctttgcgggggtgggggtgggggtgggggctgctgctgctgtggctgctcctcgtgaatccctttcttttcatcttttgcattacttttcttgctttcatcaccaacctatttctcatctgcctcctcccctactgaaacccctgttttcctattgccagagagagccatctgtcagaatatggctgtagttactatcataattcaaatattcccttttaagtatttcttgacccaacaattacaaagaagcactgctagatacaaacataaataaaatgtgctaatgaggaatgcaaacagtattaaaatgtaaacttaaaataggaaggaatttgtggccaaagaaaaactgattacgattctttcagtcttttaaaatgcatcataaaactaaaaatgttttgctacagaataacagtttggcttcttttatgatggcaaagaagggtaattacgattgtcatgaaaacacattgagcagctgtatactgttaacaaaagaaccattatatgagctcgtaaaagctggcactttatatattcacatttcctagaaattagattgtcttcccaattggagtcgataccaaaagagactgttttctattaattgtggctttgtaccagtcaattttcagagccaactggatcatctttcttttgggatatatcccacatattattatgtggaatttatatatactgatttatatacactcagaagcatacagtataatgttgcaaagagtaattttttaagttcccagagatctgtaaatttttaacgttaaaaaatgccagtttcagtagattctttcaccaacgtacatctgtttagaaattactgaaagaaatcataacgcataactctgcagtttattttatgaaggctaattaataggtacttactcattaaagactaggtctggtgcaaaatagagaaattggctgttcgtatgtttgtacgatctccagctcaaggcaaatgatgatagacacatccaagaatactggattagggtaatttggtcctcaagaggcaagtttttaaatcctgaagaacaaaacaattaatcacagaaatacacttagcatttaagtacattccaggaagatgcttcttaagtcaaccccaaacagccacctttctttcacttttctcttttctgacagactcaaatcaattcaacagtcatttacagagtgcccaccactggcccagtcctgttccacatttcactttaaattcatcctaaattctagatgagggtaaactgccagggcaaactaaaattgcaaagttcagcaagtttcagtgtggatgacagcctaaaacccttggttagattgagagaatttgtattaacctgtaaaacttgcttcgtcccacccacaattaataacactaaaataaagggtaatttaataactgtcaaaccctgtgtatctgcaataatggtgggagatttcattgttcatgag

SEQ ID NO: 78 - DNA, 9212 bp, Artificial Sequence
  Description of Artificial Sequence Synthetic pCDGal4-DBD-MR sequence
agcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccgggtacctttgtggtgcttaaaaaatgagcatgtggtgcctcctatggtccaggttaataccagactttgataaagttctatggtagaacatgtttccctggcacctgtcctccctgcctgacagactcaagctcttcttttgttctttattttatttatttattttttaagatggattctcattctgtcgcccaggctggagttcagtgacatgatattggctcactgcatcccaggtttcaagcgattctcctgcctcaggctcacaagtagctggggttacaggtgcctgccaggacgcccaactaaattttgtatttttagtagagacagggtttcatgatggtggcaaggctggtctcaaattcctgacctcaagtgatctgcttgcctcagcctcccaaagtctgggattacaggcatgggccactgcaccaggccttcttttgttttttatcacctcccccatctccaggggcatcatgttatttttctgcctgtggagtttcactgggcaagctcctcttcaaagaatcagtcagtggagctttccccaacttttacagctgctgtctgaccacaggtaatctaagaaactgctttagattaatgaactgggacaatgtaatggcaatttctaagtgatgtttctgtagattaagaggggccttattaattaaataccatgaaacaaaaaaaaatgactaaaataggaattccagggacaatttaaagggtgctgggggccaggtgattattttttagacagaagagtcattccaggtctcttacaacttacttgctaggccttgccttacttggtctacacttagattaaccctcgttatgagccgctaataatgtgctcaacttgtgatttcatgaacagaatgtattttaccttattaatatttttggatgaaattttccattttgatcaacataagaagtgtatgtcattttttttatttttaatttcttttttttttttgagacagagtctcactctgttgctcaggctggagtatagtggtgggatctcggctcactgcagcctctgccgcccaggttcaagcgattctcctgcctcagcctcctgagtagctgggattacaggcacctgccaccaagcaggactaatttttgtgtttttagtagaaacggggtttcaccatcttggctaggctggtcttgaactcctgacctcgtgatccatctgcctcagtaagccacctgccttggcctcccaaagtgctgggattacaggcgtgagccactgcgcccagccgtatctgtcattttaacaataaaatccttaaccctgcattctcggaagaaatgagcacattaaccatatttttcctatacacttgatcaatttgttatcaatttaaaaaatctgatatcaggatttcatagaacgcaaactcctcctgcatggttggagatggcgaagtcagttgcccagacttgttaagttcaggatgcagcctgtgaaaggagaggcaatcctacctggaagtacctttgcccacttcacgacttggatcatctgtttgcctgctaagcggttgagcgtggagagcagattttcggctgtatctggttttgagctgtcatagcctgcatatacaatttcaggttcaatgttttcaaggaccataacgggggaaggtgtgagcgctcgtgagattgtggagagctgaggaaccagtgctgtgttgaccgagggttcttttgcaggagcgatgtacgttgtcccttcctctgggctttgcgggggtgggggtgggggtgggggctgctgctgctgtggctgctcctcgtgaatccctttcttttcatcttttgcattacttttcttgctttcatcaccaacctatttctcatctgcctcctcccctactgaaacccctgttttcctattgccagagagagccatctgtcagaatatggctgtagttactatcataattcaaatattcccttttaagtatttcttgacccaacaattacaaagaagcactgctagatacaaacataaataaaatgtgctaatgaggaatgcaaacagtattaaaatgtaaacttaaaataggaaggaatttgtggccaaagaaaaactgattacgattctttcagtcttttaaaatgcatcataaaactaaaaatgttttgctacagaataacagtttggcttcttttatgatggcaaagaagggtaattacgattgtcatgaaaacacattgagcagctgtatactgttaacaaaagaaccattatatgagctcgtaaaagctggcactttatatattcacatttcctagaaattagattgtcttcccaattggagtcgataccaaaagagactgttttctattaattgtggctttgtaccagtcaattttcagagccaactggatcatctttcttttgggatatatcccacatattattatgtggaatttatatatactgatttatatacactcagaagcatacagtataatgttgcaaagagtaattttttaagttcccagagatctgtaaatttttaacgttaaaaaatgccagtttcagtagattctttcaccaacgtacatctgtttagaaattactgaaagaaatcataacgcataactctgcagtttattttatgaaggctaattaataggtacttactcattaaagactaggtctggtgcaaaatagagaaattggctgttcgtatgtttgtacgatctccagctcaaggcaaatgatgatagacacatccaagaatactggattagggtaatttggtcctcaagaggcaagtttttaaatcctgaagaacaaaacaattaatcacagaaatacacttagcatttaagtacattccaggaagatgcttcttaagtcaaccccaaacagccacctttctttcacttttctcttttctgacagactcaaatcaattcaacagtcatttacagagtgcccaccactggcccagtcctgttccacatttcactttaaattcatcctaaattctagatgagggtaaactgccagggcaaactaaaattgcaaagttcagcaagtttcagtgtggatgacagcctaaaacccttggttagattgagagaatttgtattaacctgtaaaacttgcttcgtcccacccacaattaataacactaaaataaagggtaatttaataactgtcaaaccctgtgtatctgcaataatggtgggagatttcattgttcatgaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagtt

SEQ ID NO: 79 - DNA, 10517 bp, Artificial Sequence
  Description of Artificial Sequence Synthetic pKI-Gal-DBD-MR sequence
atcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatgcaggtcgaaggcccggagatgaggaagaggagaacagcgcggcagacgtgcgcttttgaagcgtgcagaatgccgggtccggaggaccttcgcgcccgccccgcccctgagcccgcccctgagcccgcccccggacccaccccttcccagcctctgagcccagaaagcgaaggagccaagctgctattggccgctgccccaaaggcctacccgcttccattgctcagcggtgcttgtccatctgcacgagactagtgagacgtgctacttccatttgtcacgtcctgcacgacgcgagctgcggggcgggggggaacttcctgactaggggaggagtagaaggtggcgcgaaggggccaccaaagaagggagccggttggcgctaccggtggatgtggaatgtgtgcgaggccagaggccacttgtgatgcgccaagtgccagcggggctgctaaagcgcatgctccagactgccttgggaaaagcgcctcccctacccggtagaattcctgcagcccggggttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatccggatccactagttctagcgtttaaacttaagcttgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccgggtacctttgtggtgcttaaaaaatgagcatgtggtgcctcctatggtccaggttaataccagactttgataaagttctatggtagaacatgtttccctggcacctgtcctccctgcctgacagactcaagctcttcttttgttctttattttatttatttattttttaagatggattctcattctgtcgcccaggctggagttcagtgacatgatattggctcactgcatcccaggtttcaagcgattctcctgcctcaggctcacaagtagctggggttacaggtgcctgccaggacgcccaactaaattttgtatttttagtagagacagggtttcatgatggtggcaaggctggtctcaaattcctgacctcaagtgatctgcttgcctcagcctcccaaagtctgggattacaggcatgggccactgcaccaggccttcttttgttttttatcacctcccccatctccaggggcatcatgttatttttctgcctgtggagtttcactgggcaagctcctcttcaaagaatcagtcagtggagctttccccaacttttacagctgctgtctgaccacaggtaatctaagaaactgctttagattaatgaactgggacaatgtaatggcaatttctaagtgatgtttctgtagattaagaggggccttattaattaaataccatgaaacaaaaaaaaatgactaaaataggaattccagggacaatttaaagggtgctgggggccaggtgattattttttagacagaagagtcattccaggtctcttacaacttacttgctaggccttgccttacttggtctacacttagattaaccctcgttatgagccgctaataatgtgctcaacttgtgatttcatgaacagaatgtattttaccttattaatatttttggatgaaattttccattttgatcaacataagaagtgtatgtcattttttttatttttaatttcttttttttttttgagacagagtctcactctgttgctcaggctggagtatagtggtgggatctcggctcactgcagcctctgccgcccaggttcaagcgattctcctgcctcagcctcctgagtagctgggattacaggcacctgccaccaagcaggactaatttttgtgtttttagtagaaacggggtttcaccatcttggctaggctggtcttgaactcctgacctcgtgatccatctgcctcagtaagccacctgccttggcctcccaaagtgctgggattacaggcgtgagccactgcgcccagccgtatctgtcattttaacaataaaatccttaaccctgcattctcggaagaaatgagcacattaaccatatttttcctatacacttgatcaatttgttatcaatttaaaaaatctgatatcaggatttcatagaacgcaaactcctcctgcatggttggagatggcgaagtcagttgcccagacttgttaagttcaggatgcagcctgtgaaaggagaggcaatcctacctggaagtacctttgcccacttcacgacttggatcatctgtttgcctgctaagcggttgagcgtggagagcagattttcggctgtatctggttttgagctgtcatagcctgcatatacaatttcaggttcaatgttttcaaggaccataacgggggaaggtgtgagcgctcgtgagattgtggagagctgaggaaccagtgctgtgttgaccgagggttcttttgcaggagcgatgtacgttgtcccttcctctgggctttgcgggggtgggggtgggggtgggggctgctgctgctgtggctgctcctcgtgaatccctttcttttcatcttttgcattacttttcttgctttcatcaccaacctatttctcatctgcctcctcccctactgaaacccctgttttcctattgccagagagagccatctgtcagaatatggctgtagttactatcataattcaaatattcccttttaagtatttcttgacccaacaattacaaagaagcactgctagatacaaacataaataaaatgtgctaatgaggaatgcaaacagtattaaaatgtaaacttaaaataggaaggaatttgtggccaaagaaaaactgattacgattctttcagtcttttaaaatgcatcataaaactaaaaatgttttgctacagaataacagtttggcttcttttatgatggcaaagaagggtaattacgattgtcatgaaaacacattgagcagctgtatactgttaacaaaagaaccattatatgagctcgtaaaagctggcactttatatattcacatttcctagaaattagattgtcttcccaattggagtcgataccaaaagagactgttttctattaattgtggctttgtaccagtcaattttcagagccaactggatcatctttcttttgggatatatcccacatattattatgtggaatttatatatactgatttatatacactcagaagcatacagtataatgttgcaaagagtaattttttaagttcccagagatctgtaaatttttaacgttaaaaaatgccagtttcagtagattctttcaccaacgtacatctgtttagaaattactgaaagaaatcataacgcataactctgcagtttattttatgaaggctaattaataggtacttactcattaaagactaggtctggtgcaaaatagagaaattggctgttcgtatgtttgtacgatctccagctcaaggcaaatgatgatagacacatccaagaatactggattagggtaatttggtcctcaagaggcaagtttttaaatcctgaagaacaaaacaattaatcacagaaatacacttagcatttaagtacattccaggaagatgcttcttaagtcaaccccaaacagccacctttctttcacttttctcttttctgacagactcaaatcaattcaacagtcatttacagagtgcccaccactggcccagtcctgttccacatttcactttaaattcatcctaaattctagatgagggtaaactgccagggcaaactaaaattgcaaagttcagcaagtttcagtgtggatgacagcctaaaacccttggttagattgagagaatttgtattaacctgtaaaacttgcttcgtcccacccacaattaataacactaaaataaagggtaatttaataactgtcaaaccctgtgtatctgcaataatggtgggagatttcattgttcatgaggcggccgcttgatctgatctgagtcactgaatgggatactgtccgcaagggcgggggctattcttgtccatctgggcctacggaaccagcacccatcgccaaactcttcatcctcttcctcaatctcgctttctctctcgctcgctttttttctcttctccttttttttttttttttttttttttcccccgcaaaaggaggggagagggggtaaaaaaatgctgcactgtgcggcaaggccggtgaataaacgactcgaatccaatcaccccccgccgttcctaaagttgccttttatggctcgagtggccgctgtggcttcctataaaacccggcggcgcaacccccagccactgtcaaatcaagctaacgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagaacttgtttattgcagcttataatggttacaaataaagcaatagcatcacaaatttcacaaataaagcatttttttcactgcattctagttgtggtttgtccaaactcatcaatgtatcttatcatgtcgtggcctgcatggcccggccgcaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttccagtggatcaganaaacaatgatcccacgagagatctatagatctatagatcatgagtggaggaatgagctggcccttaatttggttttgcttgtttaaattatgatatccaactatgaaacattatcataaagcaatagtaaagagccttcagtaaagagcaggcatttatctaatcccacccccacccccacccccgtagctccaatccttccattcaaaatgtaggtactctgttctcacccttcttaacaaagtatgacagtaaaaaacttccattttagtggacatctttattgtttaatagatcatcaatttatgcagacttacagctgggatttcatcagattgccatgctgagtcaagaacagtgagtgacgaagctaaccagaggctacatacgtcagagagagagctcagcctttacagctcacttcctttctcaggcattcctttgccctcggacgagtgctggggcgtcggtttccactatcggcgagtacttctacacagccatcggtccagacggccgcgcttctgcgggcgatttgtgtacgcccgacagtcccggctccgg

SEQ ID NO: 80 - DNA, 32 bp, Artificial Sequence
  Description of Artificial Sequence Synthetic primer
gatctagagagccacaccccatgttgtctcac

SEQ ID NO: 81 - DNA, 34 bp, Artificial Sequence
  Description of Artificial Sequence Synthetic primer
gagcggccgcttgccaagggccctgtgaagcagg

SEQ ID NO: 82 - DNA, 5351 bp, Artificial Sequence
  Description of Artificial Sequence Synthetic pKI-CMV-SD sequence
tccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggaccggtccccccactcacctgccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcccgggctgcaggaattctaccgggtaggggaggcgcttttcccaaggcagtctggagcatgcgctttagcagccccgctggcacttggcgcatcacaagtggcctctggcctcgcacacattccacatccaccggtagcgccaaccggctcccttctttggtggccccttcgcgccaccttctactcctcccctagtcaggaagttcccccccgccccgcagctcgcgtcgtgcaggacgtgacaaatggaagtagcacgtctcactagtctcgtgcagatggacaagcaccgctgagcaatggaagcgggtaggcctttggggcagcggccaatagcagcttggctccttcgctttctgggctcagaggctgggaaggggtgggtccgggggcgggctcaggggcgggctcaggggcggggcgggcgcgaaggtcctccggacccggcattctgcacgcttcaaaagcgcacgtctgccgcgctgttctcctcttcctcatctccgggccttcgacctgcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgagctcgctgatcagcctcgactgtgccttctagttgccagccatctgttgtttgcccctcccccgtgccttcccattgtctgagtaggtgtcattctattctggggggtggggtggggcaggacagcaagggggaggattgggaagacaatagcaggcatgctggggatgcggtgggctctatggcttctgaggcggaaagaaccagctgggggatatcaagcttatcgataccgtcgacctcgagggggggcccggtacccagcttttgt

SEQ ID NO: 83 - DNA, 9131 bp, Artificial Sequence
  Description of Artificial Sequence Synthetic pKI-CMV-SD-Vanilloid sequence
cgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgagctcgctgatcagcctcgactgtgccttctagttgccagccatctgttgtttgcccctcccccgtgccttcccattgtctgagtaggtgtcattctattctggggggtggggtggggcaggacagcaagggggaggattgggaagacaatagcaggcatgctggggatgcggtgggctctatggcttctgaggcggaaagaaccagctgggggatatcaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgcttgccaagggccctgtgaagcagggtaccccaaaactccaacttgctgctgcccccagccccctagaactgaagtgtttccaggactgaacctctcaccccagaggcctcagaggtgagcaggcctagcagggcccaggcagtcctgcggccactgacgtcggcctgggagggtggacaggcccccggctcagagccatctgtggttctgggttagacccatccctcctcgtctctgggcctcaggtgcctcctctgcagggtgaggttgataaccaccgcgctgcctccctccccacacagaacaaggaaaacggccggggtggctgtcgtgggctgaattcagggaggatctcggtgatgaggatggctcagtccagaagagccaagcagactgtgtccctggaatagggtgagcggcagagactggcccacccacacccacaccgcactgcggggccctgctgtgcaacgggtccacacatcatcagccaggtgcccggcctgcctttgctcttcaacctcgctgccacagccactgagcttatacgagattttggattttgtgctttttgtcatttttatgaatcaacatctgtcaagcatcctggccttgaatgtttaggcgtgtgtgtgtgtgtgtgtgtgtgtgtgtctcactctgtcgcccagactagagtgcaatggggcaatctctgttcactgcaacctccacctcccaggttcaagcgattctcctccctcagcctcccaagtagctgggattacaggcgcccgccaccatgcgtggctaatgtttgtatttttagtagagacagggaacgtttaggctttaagtgatttggagctggttggtcttctaggcctaaacacctagagatcagctggccgctggcagcagcatgtgtagttggacacatgagctgatgctattttccactcccatcctgccccatccccatgcttcccctccctctggctgggcatccccactccctggcacccaggggtggcccatacatccctcccatagcctctattcccatcgggtctccacctaagggctttttcctctgtctacccttgcacaagtcctatcaccactcagatgctacttcccctcaggatccagcactgatgccgcctcctccatgaagcccccagatccctgccctgctcatgctccccgctgtcctccacacagtggcccagcccactgaccgcctgtcccacacagcctaccatgtggtccagcaattgctatctgtcactgatttatctctcccgcaccctgtgacctcctcctgggcagggacctttcctggtccatctccttatgaagaagctgtactggcctgggcgcgtggctcacgcctgtaatcccagcactttgggaggccaaggcaggtggatcacctgaggtcaggagttcgagaccagcctggccaacatggtgaaatcccatctctactaaaaaaaatacaaaaattagccgggcgtggtgtcgggcgcctgtaatcccagctactcgggaggctgaggcaggagaatcgcttgaacccaggatgtagaggttacagtgagctgagactgtgccactgcactccagcctgggcaacagagtgaaactctgtctcaaaaaaaaaaaaaaaaaaaaaaaaagaagcagcagcagcagcagcagcagcagctgcacacatggggaaataagagcaatcagacccttctaattgccatttccataggaagttagggagattactgttccagcagtggctacattccggaaaacagccgcagaagaaatgagaggcttctcaagtcctgcattttctttttttttttttttgaaatatataatcaggcaatctcccaagccagaatggctcagagagactcccaagtcctacattttcaagttaaaattcaggcagtgaaactccagagcacacaactgggccatggctgtaaaccagctacgttctgaatctcagatccatgaaaacacagaggcagcctcaccactccgcaggctgtgaacaggacggacggctggctactggccatctccctgcctgcctgttgatccactttcaacaggcctgagtctttctctgactcacccatcaggtggaccccctgcagcaggagtgttcccggccacagaaagccgattccctcatctcatcactcaatacctatctactgagtcctcctcctcagagtccagccctgcgctgggggcacagagaggagtcagctccccgctgagacagaaaccaggtactggctgggcggagggactgcaggagggggcagggtgctgaagaggggtccatggcagttggtgatggatgagctgagcctgggaagggaacagaggtgggctagggtctggagacagacagggaagcctagagcagctgaagaacgggaagtcagtgggacaggaagacaggaggccgggttagaaccagatggccgtcagccttaactccaaggaattgaaagccaacatggttgcatggatgatgctttggaagcctggggggccgggtgctagtcttccctttctgaagataacaccccgtcaaggaccgttcacagtgcggacagccctggcagcacttaaaaggcaccagaagcgaatgagaagcagaatcgttgatgcgtctgagcacgtgcacaaagcagacacggttctcaggactcgcattccttcacccacttcattcccacaacatccctaagagggaaatacttcaccgatgaggagaccgaggcacagagcagttatggcacgtccccgaggtcacacagtcagagatggaacccggacttgaccccaggcaggctggctccagagcatctccagtgctccaccacgatgctggaggtctggggctctttctcagcagttcaactcagacccagactccacagcagggcctgggggccaatcagaataggccctctcacccaaggggctaggcataagacctgaacctgaagcacctctcgctcaaacaagcagctcaggaaccccaaaatgcagtgtgaataagacccaggtcatacccatgcctggctgtcccagggatgtctatggtctccaagaataagctaagaataagaataggggctgctcgttaggcagcccagcaagagagcacctgttagccggagcctcccatgaacctttcggcaatgttctttgtcagcggcgttgccttttcctctgacgggtcccagtccaaccaaacccagccaacaggaagcagcctggattgtgctatcattcagtcagtcgacaaacgctgaatctatactgcacactctgcacctgattccctactggaaaccaagcttcttccagggaacggggtgggacaggaactgaggggtcatgtgccctgggctgggccggggaacagggtcaggacagccctccactgccaggcctggagtgtaccctgcacctaagcccgtggtgatgcacctgtcaccgcaaaacattctacagtgatcaggggaacttctaccgtgatttaggcaaacgagtgttggctgtgtgatggagatggtgattctggtttacacaagactcacaaactcatcaatctggactaaaggacaatcctaaaagaatcgattctgcccacagagacgtggtatgatctgagtctttctcaactccaaagccagaacagatactgcctaccctctgggggaaggatcccttctctccccaatgcaagtgagacaacatggggtgtggctctctagaactagtggatcccccgggaccggtccccccactcacctgccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcccgggctgcaggaattctaccgggtaggggaggcgcttttcccaaggcagtctggagcatgcgctttagcagccccgctggcacttggcgcatcacaagtggcctctggcctcgcacacattccacatccaccggtagcgccaaccggctcccttctttggtggccccttcgcgccaccttctactcctcccctagtcaggaagttcccccccgccccgcagctcgcgtcgtgcaggacgtgacaaatggaagtagcacgtctcactagtctcgtgcagatggacaagcaccgctgagcaatggaagcgggtaggcctttggggcagcggccaatagcagcttggctccttcgctttctgggctcagaggctgggaaggggtgggtccgggggcgggctcaggggcgggctcaggggcggggcgggcgcgaaggtcctccggacccggcattctgcacgcttcaaaagcgcacgtctgccgcgctgttctcctcttcctcatctccgggccttcgacctgcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatg

SEQ ID NO: 84 - DNA, 1121 bp, Artificial Sequence
  Description of Artificial Sequence Synthetic EYPFP sequence
cttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggatccttctagagtcgacgatcgaagttagcttgatttgacagtggctgggggttgcgccgccgggttttataggaagccacagcggccactcgagccataaaaggcaactttaggaacggcggggggtgattggattcgagtcgtttattcaccggccttgccgcacagtgcagcatttttttaccccctctcccctccttttgcgggggaaaaaaaaaaaaaaaaaaaaaaaaggagaagagaaaaaaagcgagcgagagagaaagcgagattgaggaagaggatgaagagtttggcgatgggtgctggttccgtaggcccagatggacaagaatagcccccgcccttgcggacagtatcccattcagtgactcagatcagatcaagc

SEQ ID NO: 85 - DNA, 10557 bp, Artificial Sequence
  Description of Artificial Sequence Synthetic pKI-CMV-SD-Vanilloid-YFP sequence
tttttaccccctctcccctccttttgcgggggaaaaaaaaaaaaaaaaaaaaaaaaggagaagagaaaaaaagcgagcgagagagaaagcgagattgaggaagaggatgaagagtttggcgatgggtgctggttccgtaggcccagatggacaagaatagcccccgcccttgcggacagtatcccattcagtgactcagatcagatcaagccggccgcttgccaagggccctgtgaagcagggtaccccaaaactccaacttgctgctgcccccagccccctagaactgaagtgtttccaggactgaacctctcaccccagaggcctcagaggtgagcaggcctagcagggcccaggcagtcctgcggccactgacgtcggcctgggagggtggacaggcccccggctcagagccatctgtggttctgggttagacccatccctcctcgtctctgggcctcaggtgcctcctctgcagggtgaggttgataaccaccgcgctgcctccctccccacacagaacaaggaaaacggccggggtggctgtcgtgggctgaattcagggaggatctcggtgatgaggatggctcagtccagaagagccaagcagactgtgtccctggaatagggtgagcggcagagactggcccacccacacccacaccgcactgcggggccctgctgtgcaacgggtccacacatcatcagccaggtgcccggcctgcctttgctcttcaacctcgctgccacagccactgagcttatacgagattttggattttgtgctttttgtcatttttatgaatcaacatctgtcaagcatcctggccttgaatgtttaggcgtgtgtgtgtgtgtgtgtgtgtgtgtgtctcactctgtcgcccagactagagtgcaatggggcaatctctgttcactgcaacctccacctcccaggttcaagcgattctcctccctcagcctcccaagtagctgggattacaggcgcccgccaccatgcgtggctaatgtttgtatttttagtagagacagggaacgtttaggctttaagtgatttggagctggttggtcttctaggcctaaacacctagagatcagctggccgctggcagcagcatgtgtagttggacacatgagctgatgctattttccactcccatcctgccccatccccatgcttcccctccctctggctgggcatccccactccctggcacccaggggtggcccatacatccctcccatagcctctattcccatcgggtctccacctaagggctttttcctctgtctacccttgcacaagtcctatcaccactcagatgctacttcccctcaggatccagcactgatgccgcctcctccatgaagcccccagatccctgccctgctcatgctccccgctgtcctccacacagtggcccagcccactgaccgcctgtcccacacagcctaccatgtggtccagcaattgctatctgtcactgatttatctctcccgcaccctgtgacctcctcctgggcagggacctttcctggtccatctccttatgaagaagctgtactggcctgggcgcgtggctcacgcctgtaatcccagcactttgggaggccaaggcaggtggatcacctgaggtcaggagttcgagaccagcctggccaacatggtgaaatcccatctctactaaaaaaaatacaaaaattagccgggcgtggtgtcgggcgcctgtaatcccagctactcgggaggctgaggcaggagaatcgcttgaacccaggatgtagaggttacagtgagctgagactgtgccactgcactccagcctgggcaacagagtgaaactctgtctcaaaaaaaaaaaaaaaaaaaaaaaaagaagcagcagcagcagcagcagcagcagctgcacacatggggaaataagagcaatcagacccttctaattgccatttccataggaagttagggagattactgttccagcagtggctacattccggaaaacagccgcagaagaaatgagaggcttctcaagtcctgcattttctttttttttttttttgaaatatataatcaggcaatctcccaagccagaatggctcagagagactcccaagtcctacattttcaagttaaaattcaggcagtgaaactccagagcacacaactgggccatggctgtaaaccagctacgttctgaatctcagatccatgaaaacacagaggcagcctcaccactccgcaggctgtgaacaggacggacggctggctactggccatctccctgcctgcctgttgatccactttcaacaggcctgagtctttctctgactcacccatcaggtggaccccctgcagcaggagtgttcccggccacagaaagccgattccctcatctcatcactcaatacctatctactgagtcctcctcctcagagtccagccctgcgctgggggcacagagaggagtcagctccccgctgagacagaaaccaggtactggctgggcggagggactgcaggagggggcagggtgctgaagaggggtccatggcagttggtgatggatgagctgagcctgggaagggaacagaggtgggctagggtctggagacagacagggaagcctagagcagctgaagaacgggaagtcagtgggacaggaagacaggaggccgggttagaaccagatggccgtcagccttaactccaaggaattgaaagccaacatggttgcatggatgatgctttggaagcctggggggccgggtgctagtcttccctttctgaagataacaccccgtcaaggaccgttcacagtgcggacagccctggcagcacttaaaaggcaccagaagcgaatgagaagcagaatcgttgatgcgtctgagcacgtgcacaaagcagacacggttctcaggactcgcattccttcacccacttcattcccacaacatccctaagagggaaatacttcaccgatgaggagaccgaggcacagagcagttatggcacgtccccgaggtcacacagtcagagatggaacccggacttgaccccaggcaggctggctccagagcatctccagtgctccaccacgatgctggaggtctggggctctttctcagcagttcaactcagacccagactccacagcagggcctgggggccaatcagaataggccctctcacccaaggggctaggcataagacctgaacctgaagcacctctcgctcaaacaagcagctcaggaaccccaaaatgcagtgtgaataagacccaggtcatacccatgcctggctgtcccagggatgtctatggtctccaagaataagctaagaataagaataggggctgctcgttaggcagcccagcaagagagcacctgttagccggagcctcccatgaacctttcggcaatgttctttgtcagcggcgttgccttttcctctgacgggtcccagtccaaccaaacccagccaacaggaagcagcctggattgtgctatcattcagtcagtcgacaaacgctgaatctatactgcacactctgcacctgattccctactggaaaccaagcttcttccagggaacggggtgggacaggaactgaggggtcatgtgccctgggctgggccggggaacagggtcaggacagccctccactgccaggcctggagtgtaccctgcacctaagcccgtggtgatgcacctgtcaccgcaaaacattctacagtgatcaggggaacttctaccgtgatttaggcaaacgagtgttggctgtgtgatggagatggtgattctggtttacacaagactcacaaactcatcaatctggactaaaggacaatcctaaaagaatcgattctgcccacagagacgtggtatgatctgagtctttctcaactccaaagccagaacagatactgcctaccctctgggggaaggatcccttctctccccaatgcaagtgagacaacatggggtgtggctctctagaactagtggatcccccgggaccggtccccccactcacctgccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcccgggctgcaggaattctaccgggtaggggaggcgcttttcccaaggcagtctggagcatgcgctttagcagccccgctggcacttggcgcatcacaagtggcctctggcctcgcacacattccacatccaccggtagcgccaaccggctcccttctttggtggccccttcgcgccaccttctactcctcccctagtcaggaagttcccccccgccccgcagctcgcgtcgtgcaggacgtgacaaatggaagtagcacgtctcactagtctcgtgcagatggacaagcaccgctgagcaatggaagcgggtaggcctttggggcagcggccaatagcagcttggctccttcgctttctgggctcagaggctgggaaggggtgggtccgggggcgggctcaggggcgggctcaggggcggggcgggcgcgaaggtcctccggacccggcattctgcacgcttcaaaagcgcacgtctgccgcgctgttctcctcttcctcatctccgggccttcgacctgcatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgagctcgctgatcagcctcgactgtgccttctagttgccagccatctgttgtttgcccctcccccgtgccttcccattgtctgagtaggtgtcattctattctggggggtggggtggggcaggacagcaagggggaggattgggaagacaatagcaggcatgctggggatgcggtgggctctatggcttctgaggcggaaagaaccagctgggggatatcaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgcaacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaagctttacttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggatccttctagagtcgacgatcgaagttagcttgatttgacagtggctgggggttgcgccgccgggttttataggaagccacagcggccactcgagccataaaaggcaactttaggaacggcggggggtgattggattcgagtcgtttattcaccggccttgccgcacagtgcagcatt

SEQ ID NO: 86 - DNA, 3799 bp, Artificial Sequence
  Description of Artificial Sequence Synthetic nucleotide sequence for Vanilloid region of homology
ttcaagcgattctcctgcctcagcctcccgagtagctgggattacaggcgcccgacaccacgcccggctaatttttgtattttttttagtagagatgggatttcaccatgttggccaggctggtctcgaactcctgacctcaggtgatccacctgccttggcctcccaaagtgctgggattacaggcgtgagccacgcgcccaggccagtacagcttcttcataaggagatggaccaggaaaggtccctgcccaggaggaggtcacagggtgcgggagagataaatcagtgacagatagcaattgctggaccacatggtaggctgtgtgggacaggcggtcagtgggctgggccactgtgtggaggacagcggggagcatgagcagggcagggatctgggggcttcatggaggaggcggcatcagtgctggatcctgaggggaagtagcatctgagtggtgataggacttgtgcaagggtagacagaggaaaaagcccttaggtggagacccgatgggaatagaggctatgggagggatgtatgggccacccctgggtgccagggagtggggatgcccagccagagggaggggaagcatggggatggggcaggatgggagtggaaaatagcatcagctcatgtgtccaactacacatgctgctgccagcggccagctgatctctaggtgtttaggcctagaagaccaaccagctccaaatcacttaaagcctaaacgttccctgtctctactaaaaatacaaacattagccacgcatggtggcgggcgcctgtaatcccagctacttgggaggctgagggaggagaatcgcttgaacctgggaggtggaggttgcagtgaacagagattgccccattgcactctagtctgggcgacagagtgagacacacacacacacacacacacacacacgcctaaacattcaaggccaggatgcttgacagatgttgattcataaaaatgacaaaaagcacaaaatccaaaatctcgtataagctcagtggctgtggcagcgaggttgaagagcaaaggcaggccgggcacctggctgatgatgtgtggacccgttgcacagcagggccccgcagtgcggtgtgggtgtgggtgggccagtctctgccgctcaccctattccagggacacagtctgcttggctcttctggactgagccatcctcatcaccgagatcctccctgaattcagcccacgacagccaccccggccgttttccttgttctgtgtggggagggaggcagcgcggtggttatcaacctcaccctgcagaggaggcacctgaggcccagagacgaggagggatgggtctaacccagaaccacagatggctctgagccgggggcctgtccaccctcccaggccgacgtcagtggccgcaggactgcctgggccctgctaggcctgctcacctctgaggcctctggggtgagaggttcagtcctggaaacacttcagttctagggggctgggggcagcagcaagttggagttttggggtaccctgcttcacagggcccttggcaagcggccgctcagatctagagagccacaccccatgttgtctcacttgcattggggagagaagggatccttcccccagagggtaggcagtatctgttctggctttggagttgagaaagactcagatcataccacgtctctgtgggcagaatcgattcttttaggattgtcctttagtccagattgatgagtttgtgagtcttgtgtaaaccagaatcaccatctccatcacacagccaacactcgtttgcctaaatcacggtagaagttcccctgatcactgtagaatgttttgcggtgacaggtgcatcaccacgggcttaggtgcagggtacactccaggcctggcagtggagggctgtcctgaccctgttccccggcccagcccagggcacatgacccctcagttcctgtcccaccccgttccctggaagaagcttggtttccagtagggaatcaggtgcagagtgtgcagtatagattcagcgtttgtcgactgactgaatgatagcacaatccaggctgcttcctgttggctgggtttggttggactgggacccgtcagaggaaaaggcaacgccgctgacaaagaacattgccgaaaggttcatgggaggctccggctaacaggtgctctcttgctgggctgcctaacgagcagcccctattcttattcttagcttattcttggagaccatagacatccctgggacagccaggcatgggtatgacctgggtcttattcacactgcattttggggttcctgagctgcttgtttgagcgagaggtgcttcaggttcaggtcttatgcctagccccttgggtgagagggcctattctgattggcccccaggccctgctgtggagtctgggtctgagttgaactgctgagaaagagccccagacctccagcatcgtggtggagcactggagatgctctggagccagcctgcctggggtcaagtccgggttccatctctgactgtgtgacctcggggacgtgccataactgctctgtgcctcggtctcctcatcggtgaagtatttccctcttagggatgttgtgggaatgaagtgggtgaaggaatgcgagtcctgagaaccgtgtctgctttgtgcacgtgctcagacgcatcaacgattctgcttctcattcgcttctggtgccttttaagtgctgccagggctgtccgcactgtgaacggtccttgacggggtgttatcttcagaaagggaagactagcacccggccccccaggcttccaaagcatcatccatgcaaccatgttggctttcaattccttggagttaaggctgacggccatctggttctaacccggcctcctgtcttcctgtcccactgacttcccgttcttcagctgctctaggcttccctgtctgtctccagaccctagcccacctctgttcccttcccaggctcagctcatccatcaccaactgccatggacccctcttcagcaccctgccccctcctgcagtccctccgcccagccagtacctggtttctgtctcagcggggagctgactcctctctgtgcccccagcgcagggctggactctgaggaggaggactcagtagataggtattgagtgatgagatgagggaatcggctttctgtggccgggaacactcctgctgcagggggtccacctgatgggtgagtcagagaaagactcaggcctgttgaaagtggatcaacaggcaggcagggagatggccagtagccagccgtccgtcctgttcacagcctgcggagtggtgaggctgcctctgtgttttcatggatctgagattcagaacgtagctggtttacagccatggcccagttgtgtgctctggagtttcactgcctgaattttaacttgaaaatgtaggacttgggagtctctctgagccattctggcttgggagattgcctgattatatatttcaaaaaaaaaaaaaaagaaaatgcaggacttgagaagcctctcatttcttctgcggctgttttccggaatgtagccactgctggaacagtaatctccctaacttcctatggaaatggcaattagaagggtctgattgctcttatttccccatgtgtgcagctgctgctgctgctgctgctgctgcttctttttttttttttttttttttttttgagacagagtttcactctgttgcccaggctggagtgcagtggcacagtctcagctcactgtaacctctacatcctggg

(19) United States
(12) Patent Application Publication
(10) Pub. No.: US 2005/0003367 A1
(43) Pub. Date: Jan. 6, 2005

(54) Methods and compositions for rapid development of screening assays

(76) Inventors: Michael Allen Whitney, San Diego, CA (US); Pamela Susan Sanders, San Diego, CA (US); Karin Zeh, San Diego, CA (US)

Correspondence Address: Nandakumar Govindaswamy;VERTEX PHARMACEUTICALS INCORPORATED, 130 Waverly Street, Cambridge, MA, 02139-4242, US

(21) Appl. No.: 10/656,029

(22) Filed: Sep. 5, 2003

Related U.S. Application Data
(60) Provisional application No. 60/408,297, filed on Sep. 5, 2002.

(51) Int. Cl. C12Q 1/68; C12N 15/85

(52) U.S. Cl. 435/6; 435/455

(57) ABSTRACT

The present invention relates to methods for creating a sensor cell, sensor cells and the use of those cells to assay the ability of a test compound to modulate the activity of a target gene and/or its expression product. The methods and cells of this invention utilize homologous recombination to control expression of an activatable domain of a target gene and a signal transduction system to detect activation of that expression product. The invention also relates to DNA sequences that are useful to in the homologous recombination steps of the methods of this invention.

This application claims priority to U.S. Provisional Patent Application Ser. No. 60/408,297 filed on Sep. 5, 2002 and incorporated herein by reference.

### TECHNICAL FIELD OF THE INVENTION

The present invention relates to methods for creating a sensor cell, sensor cells and the use of those cells to assay the ability of a test compound to modulate the activity of a target gene and/or its product. The methods and cells of this invention utilize homologous recombination to control expression of an activatable domain of a target gene which, when activated, causes a detectable change in a signal transduction detection system. The methods and cells can be utilized to measure the effect of modulating such expression and/or activation by test compounds. The invention also relates to DNA sequences that are useful to in the homologous recombination steps of the methods of this invention.

### BACKGROUND OF THE INVENTION

The identification and functional analysis of proteins involved in disease processes requires extensive expenditure of time and financial resources. Currently used strategies use cloning and expression to develop in vitro cellular or biochemical assays designed to detect the activity of the protein of interest and the effect of test compounds on that activity. Such approaches typically require access to and expression of full-length cDNAs, purification of the expressed protein in an active form, and knowledge of the mechanism of action of the protein of interest so that an appropriate activity assay can be set up.

Accordingly, these approaches are not readily applicable to orphan gene products that do not possess well characterized mechanisms of action, nor to gene products that are difficult to produce recombinantly in sufficiently high yield and in an active form. Many such targets may also be difficult to clone by traditional approaches, or are difficult to express with the correct, physiologically relevant characteristics. Additionally, many enzymes are encoded by very long mRNAs that are unstable in cloning vectors or otherwise difficult to work with and characterize.

Thus, there exists a need for simple and robust ways of rapidly developing assays for novel drug targets that simplify the cloning process, do not require extensive knowledge or analysis of the gene, and do not require an in depth analysis of the mechanism of action of the protein of interest.

Nuclear receptors are a class of ligand-regulated transcription factors that fit within the general category of genes that can exert complex biological responses through multiple signal transduction activation pathways. This can complicate the development of in vitro assays capable of measuring nuclear receptor function.

Nuclear receptors provide multi-cellular organisms with a means to directly control gene expression in response to a wide range of developmental, physiological and environmental cues, and have been successfully targeted for drug discovery. Specific examples of drugs targeted at nuclear receptors include Avanida® (rosiglitazone) and Actos® (pioglitazone) marketed by GlaxoSmithKline and Takeda Pharma, respectively. These two drugs target the function of the peroxisome proliferator associated receptor-γ (PPARγ) nuclear receptor and are used as insulin sensitizers for the treatment of type II diabetes.

Interest in the nuclear receptor family has grown recently with the realization that this class of molecules has served as outstanding targets for the discovery of new drugs. It is now believed that there are about 48 members of the nuclear receptor superfamily in humans. Furthermore, many of the newly identified nuclear receptors are called orphan receptors since their native physiological ligand and associated biological effects are presently unknown. Because of the known importance of the non-orphan or de-orphanized members of the nuclear receptor superfamily, orphan receptors potentially represent an important new source of viable targets for drug discovery.

In general nuclear receptors are composed of four independent functional modules. These are the modulator domain (ligand independent activation domain), the DNA binding domain (DBD), the hinge region, and the ligand binding domain (LBD) (ligand dependent activation domain) (FIG. 1). In some cases the sequence of the protein extends beyond the LBD at the carboxy-terminal end.

The modulator domain, which is sometimes also referred to as the A/B domain, displays a high degree of variability both in terms of length and primary sequence. In many cases alternative splicing, transcription from different promoters, and distinct translational start sites leads to the generation of multiple modulator domains, leading to the expression of many receptor isoforms from a single gene.

The modulator domain usually contains a transcriptional activation function, often referred to as AF-1 that mediates its activity by interactions with other members of the cellular transcription machinery. There are slight to non-detectable amino acid residue sequence homologies between members of the nuclear recptor superfamily within this domain

The DNA binding domains (DBDs) of nuclear receptors are the most conserved protein domains between family members. Typical DBDs comprise two zinc finger modules of about 66 to 70 amino acids each and a carboxyl -terminal extension (CTE) of about 25 amino acids. Individual superfamily DBDs may bind to DNA as a monomer, homodimer or heterodimer. Most heterodimeric complexes contain one of the Retinoid X receptors (RXRs) as a common partner. Direct recognition of the DNA is mediated by specific sub-domains (P-box) within the DBD as are dimerization sub-domains. The CTE plays dual roles in providing both protein-DNA and protein-protein interfaces.

Nuclear receptor DNA binding domains recognize specific nucleotide sequences, typically referred to as response elements (“REs”), within the chromosomal DNA. Response elements typically comprise one or two consensus core half-site sequences. For dimeric REs, the half sites can be configured as inverted, everted or direct repeats. For monomeric REs, a single half-site is preceded by a 5′ flanking A/T rich sequence. Half site sequences can deviate quite considerably from the consensus sequences, especially for dimeric REs in which a single conserved half site is usually sufficient to confer high affinity binding to the homo or heterodimer complexes. It is noteable that REs rarely contain two perfect consensus half sites, implying a degree of flexibility in the sequence-specific recognition of REs by nuclear recpetors.

The hinge region is highly variable both in length and primary sequence. Its main function serves to align the nuclear receptor DBD to the LBD. The hinge region also flexes to enable the DBD to rotate 180 degrees, allowing some nuclear receptors to bind as dimers to direct or inverted REs. The hinge region may also serve as a docking site for co-repressor proteins.

The nuclear receptor ligand binding domain functions to mediate ligand binding, dimerization heat shock protein interactions, nuclear localization, and transactivation functions. Nuclear receptor LBDs can be defined by a signature motif that overlaps with helix 4 in the structure of the protein. In addition, ligand dependent transactivation is dependent on the presence of a highly conserved activation function-2 motif (AF-2) localized at the carboxy-terminal end of the LBD. X-ray crystallographic experiments suggest that LBDs share a similar overall structure based around the folding of 11 to 13 helices into three layers that bury the ligand binding site within the core of the LBD. This restructuring reorients the terminal LBD α-helix, forming a pocket capable of recruiting transcriptional activators including steroid receptor co-activator (SRC)-1, -2, and -3, CREB binding protein (CBP) and other p300 transcriptional activators family members.

Because of the medical importance of orphan nuclear receptors and the role they play in regulating gene expression there is a need for screening assay development in order to develop new and novel therapeutic agents directed to these targets. Such methods should be feasible even in the absence of the known natural ligands, or an understanding of the specific signal transduction pathways engaged by individual nuclear receptors.

### SUMMARY OF THE INVENTION

The present invention solves the problems set forth above by utilizing: 1) homologous recombination techniques to direct the insertion of a heterologous promoter into a cell to drive expression of all or a portion of the target gene of interest; and 2) a signal transduction detection system also present in that cell which specifically responds to an activated form of that expression product and which produces an easily detected signal.

Prior to the applicants invention such approaches were not readily feasible because the screening methods traditionally used to characterize and select the responsive cells after recombination were relatively slow, time consuming and not readily applicable to detecting transient activation events on a large scale. Accordingly, homologous recombination mediated in situ gene modification has not previously been adopted as a method of generating cell based assays for drug discovery. The present inventors have solved this problem by coupling high throughput optical analysis and cellular selection to the identification of cells that have successfully incorporated new genetic elements by homologous recombination. Such cell-based assays have immediate industrial applicability for the identification of therapeutic agents as well as in the development of specific reagents for use in diagnostic and clinical analysis.

Furthermore such approaches use the endogenous genomic sequences that are present within the native cellular context, and do not require the use of full-length target gene cDNA clones, significantly simplifying the assay development process.

In a preferred aspect of the invention, the homologous recombination event replaces a portion of the native target gene with a DNA sequence encoding a heterologous domain. This results in an expressed product that is a fusion protein comprising the heterologous domain and an activatable domain that is homologous to the activatable domain encoded by the native target gene. In this embodiment, the heterologous domain is responsible for interacting with the signal transduction detection system following activation of the expression product. The advantages of this embodiment are that one need not know what domain of the target gene is responsible for interacting with the signal transduction system and the same signal transduction system can be used to assay multiple target gene products.

This preferred embodiment is particularly useful for assaying members of the nuclear receptor super family whose function is not yet known. This exploits the well-characterized genomic structure of the nuclear receptor superfamily and the ability to exchange functional protein domains within members of the nuclear receptor super family in situ. The invention can be applied across the entire protein class, as it is capable of producing novel cell based assays wherein the activation of an entire gene family can be monitored through a single common signal detection pathway. Thus the present invention provides for significant improvements in efficiency, particularly when developing several nuclear receptor assays in parallel.

In one aspect the present invention relates to a method of developing a sensor cell for determining the activity of a target gene in said cell comprising the steps of:
a) providing a homogeneous population of cells, wherein each of said cells comprises a signal transduction detection system,
b) introducing into said population of cells an isolated DNA construct comprising a promoter operatively linked to a targeting sequence, wherein:
   (i) said targeting sequence comprises a region of homology to said target gene sufficient to promote recombination of said DNA construct in said cells;
   (ii) said promoter is heterologous to said target gene;
   (iii) following said recombination said promoter controls transcription of a mRNA encoding a polypeptide comprising an activatable domain; and
   (iii) said-polypeptide is capable, upon activation of said activatable domain, of altering the signal detected from said signal transduction detection system,
c) incubating said population of cells under conditions which cause expression of said polypeptide;
d) incubating said population of cells under conditions which cause activation of said activatable domain of said polypeptide; and
e) selecting cells that have altered the signal detected from said signal transduction detection system.

According to another embodiment, the invention provides a recombinant sensor cell that is either made by the method described above or is equivalent to a cell made by that method.

In another aspect of this invention, a method of determining the activity of a target gene product or the effect of a test compund on such activity is provided. Such methods utilize the cells of the present invention.

In yet another embodiment, the invention provides DNA sequences that are useful in methods of creating the sensor cells of this invention.

### BRIEF DESCRIPTION OF FIGURES

FIG. 1 shows in panel A, the typical structure components and arrangement of a nuclear receptor, and in panel B shows a typical intron/exon structure for the genomic organization of a typical nuclear receptor.

FIG. 2 shows in panels A, B and C, three consecutive rounds of FACS sorting to enrich for cells expressing the MCR4 gene. Panel D shows the normalized responses for 10 clonal cell lines isolated after FACS selection in response to no stimuli (white bars) or in the presence of NDP-α-MSH (100 nM).

FIG. 3 shows the results of PCR amplication of genomic DNA extracted from the cell lines shown in FIG. 2 above. The results demonstrate the specific amplification of a band of the correct molecular weight consistent with the homologous recombination of the DNA construct into the MCR4 gene.

FIG. 4 shows dose response profiles for 3 cell clones isolated after FACS selection treated with various concentrations of NDP-α-MSH.

FIG. 5 shows the results of a mock high throughput screen using the clone MC4.49 in a 3456 well nanowell plate format.

FIG. 6 shows in panel A the results of a FACS sort of a transformed library to select for EYFP negative cells. Panels B, C and D show conseceutive FACS sorts to enrich for cells specifically responsive to PPAR gamma stimulation.

FIG. 7 shows a dose response profile for one of the clones isolated after FACS selection, clone PPARg 4G5, in response to the agonists, rosigilitazone and troglitazone.

FIG. 8 shows a dose response profile for one of the clones isolated after FACS selection, clone PPARg 4G5, in response to the various concentrations of the antagonist BADGE.

FIG. 9 shows the results of a dose response curve using the clone PPARg 4G5 in a 3456 well nanowell plate format.

FIG. 10 shows the Nurr1/RXR FACS sorting strategy.

FIG. 11 shows the 9-cis RA dose response profile of Nurr1 clones.

FIG. 12 shows the molecular validation of Nurr1 clones.

FIG. 13 shows the HEK/UAS/GAL4-Nurr1 clone, 1E10.

FIG. 14 shows the GR FACS sorting strategy.

FIG. 15 shows the Dexamethasone dose response profile of GR clones.

FIG. 16 shows the HEK/UAS/GAL4-GR clone, 2F8.

FIG. 17 shows a comparison of several sorted clones for response to aldosterone.

FIG. 18 shows the HEK/UAS/GAL4-MR clone, 1B4.

FIG. 19 shows antagonism of response to aldosterone by spironolactone. Aldosterone dose response was used to determine in the presence of indicated concentration of spironolactone (A), with results used to determine pA2 by Schild regression.

FIG. 20 shows nucleic acid sequence for the Nurr1 Targeting Sequence (SEQ ID NO:71).

FIGS. 21a to 21d shows the nucleic acid sequence for pCDGal4-DBD-Nurr1 (SEQ ID NO:72).

FIGS. 22a to 22d shows the nucleic acid sequence for pKI-Gal4-DBD-Nurr1 (SEQ ID NO:73).

FIG. 23 shows the nucleic acid sequence for GR region of homology (SEQ ID NO:74).

FIGS. 24a to 24d shows the nucleic acid sequence for pCDGal4-DBD-GR (SEQ ID NO:75).

FIGS. 25a to 25e shows the nucleic acid sequence for pKI-Gal4-DBD-GR (SEQ ID NO:76).

FIGS. 26a to 26b shows the nucleic acid sequence for MR region of homology (SEQ ID NO:77).

FIGS. 27a to 27d shows the nucleic acid sequence for pCDGal4-DBD-MR (SEQ ID NO:78).

FIGS. 28a to 28d shows the nucleic acid sequence for pKI-Gal4-DBD-MR (SEQ ID NO:79).

FIG. 29 shows capsaicin concentration-dependent calcium response of nine clones selected for final clone selection. Clones 5B5 and 5B11 exhibited the largest responses. The EC₅₀ for capsaicin against clone 5B11 was 400 nM using unoptimized assay conditions.

FIG. 30 shows capsaicin concentration-dependent calcium responses of VR1 expressed in HEK293 by homologous recombination in the presence and absence of capsazepine. Clone 5B5 EC₅₀ was 167 nM and clone 5B11 EC₅₀ was 106 nM.

FIG. 31 shows concentration-dependent responses of capazepine, a VR1 antagonist in a 96-well plate format for clone 5B11 and clone 5B5

FIG. 32 shows Capasaicin concentration-dependent calcium responses of VR1 expressed in HEK293 cells by homologous recombination. Capsaicin (squares) EC₅₀ was 103 nM and resiniferatoxin (circles) EC₅₀ was 427 nM; in 96-well plate format.

FIG. 33 shows the nucleic acid sequence for Vanilloid Region of Homology (SEQ ID NO:86).

FIG. 34 shows the nucleic acid sequence for eYPFP (SEQ ID NO:84).

FIG. 35 shows the nucleic acid sequence for pKI-CMV-SD (SEQ ID NO:82).

FIG. 36 shows the nucleic acid sequence for pKI-CMV-SD-Vanilloid (SEQ ID NO:83).

FIG. 37 shows the nucleic acid sequence for pKI-CMV-SD-Vanilloid-YFP (SEQ ID NO:85).

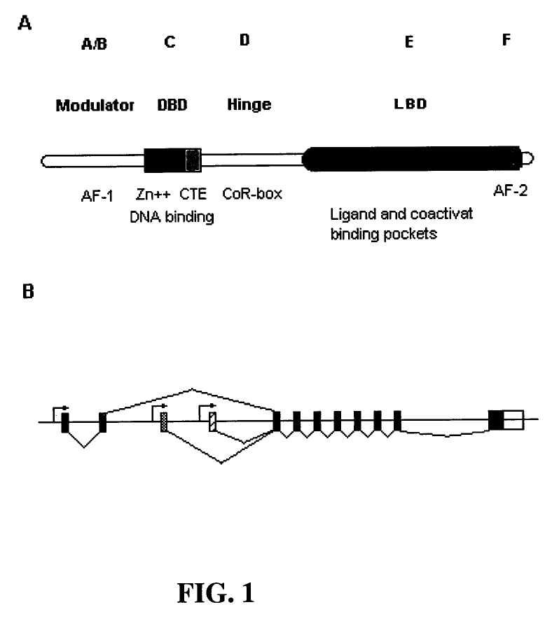

### DETAILED DESCRIPTION OF THE INVENTION

Generally, the nomenclature used herein and many of the fluorescence, computer, detection, chemistry, and laboratory procedures described below are those well known and commonly employed in the art. Standard techniques are usually used for chemical synthesis, fluorescence, optics, molecular biology, computer software, and integration. Generally, chemical reactions, cell assays and enzymatic reactions are performed according to the manufacturer's specifications where appropriate. The techniques and procedures are generally performed according to conventional methods in the art and various general references, including those listed below, which are herein incorporated by reference.

Lakowicz, J. R. “Topics in Fluorescence Spectroscopy,” (3 volumes) New York: Plenum Press (1991); Lakowicz, J. R. Emerging applications of fluorescence spectroscopy to cellular imaging: lifetime imaging, metal-ligand probes, multi-photon excitation and light quenching. Scanning Microsc. Suppl. Vol. 10, pages 213-24 (1996); Sambrook et al. “Molecular Cloning: A Laboratory Manual, 2^{nd} edition,” (1989) Cold Spring Harbor Laboratory Press, Cold Spring Harbor, N.Y.; “Cells: A Laboratory Manual, 1^{st} edition” (1998) Cold Spring Harbor Laboratory Press, Cold Spring Harbor, N.Y.; Optics Guide 5 (1990), Melles Griot and Co; A. W. Snyder and J. D. Love, “Optical Waveguide Theory,” (1983) Chapman and Hall, London.

According to one embodiment, the present invention provides a method of developing a sensor cell for determining the activity of a target gene in said cell, comprising the steps of:
a. providing a homogeneous population of cells, wherein each of said cells comprises a signal transduction detection system,
b. introducing into said population of cells an isolated DNA construct comprising a promoter operatively linked to a targeting sequence, wherein:
   i. said targeting sequence comprises a region of homology to said target gene sufficient to promote recombination of said isolated DNA construct;
   ii. said promoter is heterologous to said target gene;
   iii. following said recombination said promoter controls transcription of a mRNA encoding a polypeptide comprising an activatable domain; and
   iv. said polypeptide is capable, upon activation of said activatable domain, of altering the signal detected from said signal transduction system,
c. incubating said population of cells under conditions which cause expression of said polypeptide;
d. incubating said population of cells under conditions which cause activation of said activatable domain of said polypeptide; and
e. selecting cells that have altered the signal detected from said signal transduction system.

The term “target gene” as used herein refers to an endogenous gene in the cell, whether or not expressed at detectable levels in that cell, the activity of which is desired to be assayed. The “target gene” utilized in this invention typically codes upon expression for a polypeptide that comprises an activatable domain. The term “activatable domain” refers to a portion of a polypeptide that interacts with another substance within the cell or added to the cell or is otherwise modified, wherein following such interaction or modification (i.e., activation), the polypeptide is capable of directly or indirectly affecting the signal output of a signal transduction detection system present in the cell. Such activatable domains include, but are not limited to, ligand binding domains, domains containing phosphorylation and dephosphorylation sites, domains containing sulfation desulfation sites, domains capable of forming heteromultimers and homomultimers, domains containing glycosylation or deglycosylation sites, domains containing lipidation or delipidation sites, translocation (export or import) or subcellular targeting domains and protein degradation sequences.

In another embodiment, the endogenous target gene constitutively affects the signal output of a signal transduction detection system in the cell. In this embodiment, the target gene (or the portion thereof that is responsible for affecting that signal output) is placed under the control of an inducible promoter following recombination. The resulting gene is then considered “activatable,” as that term is used herein, in that induction of the promoter and concomitant expression of the encoded polypeptide “activates” the polypeptide and allows it to affect the signal output referred to above.

Preferably the genomic information for the target gene is readily available from genomic databases such as NCBI or ENSMBL. Preferred target genes are those genes directly involved in biological processes or signal transduction pathways. Such targets genes when expressed within cells give rise to proteins that play a significant role in the physiology or biology of an organism, and are typically directly or indirectly associated with a disease state or disease progression. Even more preferred are target genes that encode transcription factors, and particularly transcription factors comprising zinc finger DNA binding domains. Particularly preferred within this group are members of the nuclear receptor super family and most preferred are those target genes listed in Table 1.

TABLE 1

| Receptor name and | | |
|---|---|---|
| Subtype | Alternative Names | Accession no. |
| Group 0: | | |
| Adrenal hypoplasia protein, | AHC, DAX-1, NR0B1 | XM_010297 |
| Small heterodimer partner | SHP, NR0B2 | NM_021969 |
| Group 1: | | |
| Thyroid hormone receptor-α, | TRA1, NR1A1 | NM_003250 |
| high affinity | | |
| Thyroid hormone receptor-β | TRB1, NR1A2 | XM_002986 |
| Retinoic acid receptor-α | RAR-α, NR1B1 | NM_000964 |
| Retinoic acid receptor-β | RAR-β, NR1B2 | XM_053323 |
| Retinoic acid receptor-γ | RAR-γ, NR1B3 | XM_029728 |
| Peroxisome proliferator activated | PPARα, NR1C1 | NM_005036 |
| receptor-α | | |
| Peroxisome proliferator activated | PPARδ, PPARβ, NR1C2 | XM_004285 |
| receptor-δ | | |
| Peroxisome proliferator activated | PPARγ, NR1C3 | XM_003059 |
| receptor-γ | | |
| Thyroid hormone receptor, α- | EAR1, REV-ERBA, NR1D1, | NM_021724 |
| like | REV-ERB-β, BD73, NR1D2 | L31785 |
| RAR-related orphan receptor-A | ROR-A, NR1F1 | NM_002943 |
| RAR-related orphan receptor-B | ROR-B, ROR-β, NR1F2 | NM_006914 |
| RAR-related orphan receptor-C | ROR-C, ROR-γ, NR1F3 | NM_005060 |
| RAR-related orphan receptor-γt | ROR-γt, NR1F3 | |
| Liver X receptor-β | LXR-B, NR1H2 | XM_046419 |
| Liver X receptor-α | LXRA, NR1H3 | NM_005693 |
| Farnesyl X receptor | FXR, NR1H4 | NM_005123 |
| Vitamin D receptor | VDR, NR1I1 | XM_007046 |
| Pregnane X-receptor | PXR, NR1I2 | NM_003889 NM_022002 |
| | | AF364606 |
| Constitutive androstane receptor | CAR, NR1I3 | XM_042458 |
| Group 2: | | |
| Hepatocyte nuclear factor 4 alpha | HNF4-α, NR2A1 | NM_000457 |
| Hepatocyte nuclear factor 4 | HNF4-γ, NR2A2 | NM_0004133 |
| gamma | | |
| Retinoid X-receptor-α | RXRα, NR2B1 | NM_002957 |
| Retinoid X-receptor-β | RXRβ, NR2B2 | XM_042579 |
| Retinoid X-receptor-γ | RXRγ NR2B3 | XM_053680 |
| Thyroid hormone receptor-2 | TR2, NR2C1 | NM_003297 |
| Nuclear hormone receptor TR4 | TR4, NR2C2 | XM_042906 |
| Tailless (drosp.) homologue | TLL, TLX, NR2E1, | AF220532 |
| | PNR, ESCS, NR2E3 | NM_016346 |
| Transcription factor COUP-1 | EAR3, NR2F1 | NM_005654 |
| Transcription factor COUP-2 | NR2F2 | NM_021005 |
| ERBA-related gene-2 | EAR-2, NR2F6 | XM_008855 |
| Group 3: | | |
| Estrogen Receptor-α | ER-α, NR3A1, | XM_045967 |
| Estrogen Receptor-β | ER-β, NR3A2 | NM_001437 |
| Estrogen-Related Receptor-1 | ERR-1, NR3B1 | XM_048286 |
| Estrogen-Related Receptor-2 | ERR-2, NR3B2 | XM_041087 |
| Estrogen-Related Receptor-3 | ERR-3, NR3B3 | XM_039053 |
| Glucocorticoid Receptor | GR, NR3C1 | NM_000176 |
| Mineralocorticoid Receptor | MR, NR3C2 | XM_055775 |
| Progesterone Receptor | PR, NR3C3 | NM_000926 |
| Androgen Receptor | AR, NR3C4 | XM_010429 |
| Group 4: | | |
| Nerve growth factor-induced | NGFI-B/Nur77, NR4A1 | XM_083884 |
| transcript-B | | |
| Nur-related receptor-1 | Nurr1, NR4A2 | NM_006186 |
| Neuronal orphan receptor-1 | NOR-1, NR4A3 | XM_037370 |
| Group 5: | | |
| Steridogenic factor-1 | SF-1, NR5A1 | NM_004959 |
| LRH-1 | NR5A2 | XM_036634 |
| Group 6: | | |
| Germ Cell Nuclear Factor | GCNF, NR6A1 | XM_056232 |

As used herein the terms “PPAR gamma”, “PPAR g”, or “PPARγ” refers to Peroxisome Proliferator Activated Receptor-γ.

An embodiment of the present invention provides a recombinant cell line designated as HEK-293 MC4 c49 P4 ACD#12591 that is useful for identifying compounds that modulate Melanocortin Receptor (MC4R) activity. A deposit of the present cell line was made with the American Type Culture Collection, 10801 University Park, Manassas, Va. 20110-2209 on Aug. 19, 2003 and assigned ATCC Accession No. PTA-5409.

Another embodiment of the present invention provides a method of identifying compounds that modulate MC4R activity using the Cell Line designated by ATCC Accession No. PTA-5409.

A preferred aspect of this embodiment provides for the use of the Cell Line designated by ATCC Accession No. PTA-5409 for identifying compounds that modulate MC4R activity in a high throughput screen.

An embodiment of the present invention provides a recombinant cell line designated as HEK-293 PPARγ c4G5 P9 ACD#13607 that is useful for identifying compounds that modulate Peroxisome Proliferator-Activated Receptor gamma (PPARγ) activity. A deposit of the present cell line was made with the American Type Culture Collection, 10801 University Park, Manassas, Va. 20110-2209 on Aug. 19, 2003 and assigned ATCC Accession No. PTA-5405.

Another embodiment of the present invention provides a method of identifying compounds that modulate PPARγ activity using the Cell Line designated by ATCC Accession No. PTA-5405.

A preferred aspect of this embodiment provides for the use of the Cell Line designated by ATCC Accession No. PTA-5405 for identifying compounds that modulate PPARγ activity in a high throughput screen.

An embodiment of the present invention provides a recombinant cell line designated as HEK-293 GR c2F8 P5 ACD#13609 that is useful for identifying compounds that modulate Glucocorticoid receptor (GR) activity. A deposit of the present cell line was made with the American Type Culture Collection, 10801 University Park, Manassas, Va. 20110-2209 on Aug. 19, 2003 and assigned ATCC Accession No. PTA-5407.

Another embodiment of the present invention provides for a method of identifying compounds that modulate Glucocorticorticoid activity using the Cell Line designated by ATCC Accession No. PTA-5407.

Another embodiment of the present invention provides a method of identifying compounds that modulate Glucocorticorticoid receptor activity using the Cell Line designated by ATCC Accession No. PTA-5407.

A preferred aspect of this embodiment provides for the use of the Cell Line designated by ATCC Accession No. PTA-5407 for identifying compounds that modulate Glucocorticoid receptor activity in a high throughput screen.

An embodiment of the present invention provides a recombinant cell line designated as HEK-293 MR c1B4 PS ACD#13687 that is useful for identifying compounds that modulate Mineralocorticoid receptor (MR) activity. A deposit of the present cell line was made with the American Type Culture Collection, 10801 University Park, Manassas, Va. 20110-2209 on Aug. 19, 2003 and assigned ATCC Accession No. PTA-5408.

Another embodiment of the present invention provides a method of identifying compounds that modulate Mineralocorticorticoid receptor activity using the Cell Line designated by ATCC Accession No. PTA-5408.

A preferred aspect of this embodiment provides for the use of the Cell Line designated by ATCC Accession No. PTA-5408 for identifying compounds that modulate Mineralocorticoid receptor activity in a high throughput screen.

An embodiment of the present invention provides a recombinant cell line designated as HEK-293 Nurr1 c1E10 P7 ACD#13608 that is useful for identifying compounds that modulate Nurr1 receptor (Nurr1) activity. A deposit of the present cell line was made with the American Type Culture Collection, 10801 University Park, Manassas, Va. 20110-2209 on Aug. 19, 2003 and assigned ATCC Accession No. PTA-5406.

Another embodiment of the present invention provides a method of identifying compounds that modulate Nurr 1 receptor activity using the Cell Line designated by ATCC Accession No. PTA-5406.

A preferred aspect of this embodiment provides for the use of the Cell Line designated by ATCC Accession No. PTA-5406 for identifying compounds that modulate Nurr 1 receptor activity in a high throughput screen.

An embodiment of the present invention provides a recombinant cell line designated as HEK-293 C5B11 VR1 ACD#411 that is useful for identifying Vanilloid Receptor-1 Antagonist. A deposit of the present cell line was made with the American Type Culture Collection, 10801 University Park, Manassas, Va. 20110-2209 on ______ 2003 and assigned ATCC Accession No. ______.

Another embodiment of the present invention provides a method of identifying Vanilloid Receptor-1 antagonist using the Cell Line designated by ATCC Accession No. ______.

A preferred aspect of this embodiment provides for the use of the Cell Line designated by ATCC Accession No. ______ for identifying Vanilloid Receptor-1 antagonist in a high throughput screen.

The term “homogeneous population of cells,” as used herein refers to a cell line or a collection of cells whose similarity to one another, both in genotype and phenotype, is equivalent to the similarity between individual cells that exists in a typical cell line.

The choice of cells to utilize in this method is governed by two factors. First, the cells must possess the target gene of interest in its genome. Second, the cells must also possess a competent signal transduction detection system that is affected by the activated polypeptide whose expression is controlled by the heterologous promoter following recombination.

Many cell types can be used with the invention, including both primary and cultured cell lines derived from eukaryotic or prokaryotic cells that might or might not be immortalized and/or transformed. Preferably the cell line used will not express the target gene at significant levels endogenously. The preferred cell line will be from an organism for which the genome has been sequenced or the genomic sequence of the target gene is available in commonly used databases (e.g., NCBI). Such cells include, but are not limited to mammalian adult, fetal, or embryonic cells. These cells can be derived from the mesoderm, ectoderm, or endoderm and can be stem cells, such as embryonic or adult stem cells, or adult precursor cells. The cells can be of any lineage, such as vascular, neural, cardiac, fibroblasts, lymphocytes, hepatocytes, cardiac, hematopoeitic, pancreatic, epidermal, myoblasts, or myocytes. Other cells include baby hamster kidney (BHK) cells (ATCC NO. CCL10), mouse L cells (ATCC NO. CCLI.3), Jurkats (ATCC NO. TIB 152) and 153 DG44 cells (see, Chasin Cell. Molec. Genet., 12, p. 555 (1986)) human embryonic kidney (HEK) cells (ATCC NO. CRL1573), Chinese hamster ovary (CHO) cells (ATCC Nos. CRL9618, CCL61, CRL9096), PC12 cells (ATCC NO. CRL17.21) and COS-7 cells (ATCC NO. CRL1651).

Preferred established culture cell lines include Jurkat cells, CHO cells, neuroblastoma cells, P19 cells, F11 cells, NT-2 cells, and HEK 293 cells, such as those described in U.S. Pat. No. 5,024,939 and by Stillman et al., Mol. Cell. Biol., 5, pp. 2051-2060 (1985).

The term “signal transduction detection system,” as used herein refers to a protein and/or process in a cell that is affected in a measurable way by the expression and activation of the polypeptide whose expression is controlled by the heterologous promoter following homologous recombination. As detailed below, many different types of signal transduction detection systems can be utilized in the present invention. These include, but are not limited to reporter gene detection systems, change in membrane potential detection systems, post-translational modification detection systems and ionic change detection systems. Preferably such systems provide for the sensitive, rapid, detection of the activation of the target gene in a single living cell and are not cytotoxic. Preferred signal transduction systems include those that are compatible with high throughput screening, miniaturization, optical selection, and FACS analysis and provide for an optical readout. With respect to high throughput, it is preferable that the signal transduction detection system be capable of being used to screen more than 10 single living cells/second, more preferably greater than 100 living cells/second and most preferably greater than 1000 living cells/second.

The term “promoter” as used herein refers to a non-translated segment of DNA that controls the transcription and translation of a coding sequence to which it is operably linked. Promoters useful in the present invention include both constitutive and inducible promoters. It is preferred that the promoters used herein drive expression at a higher rate than the native promoter does with respect to the target gene. Examples of specific promoters useful in this invention include, but are not limited to, the herpes simplex thymidine kinase promoter, cytomegalovirus (CMV) promoter, SV40 promoter/enhancer, PGA promoter, regulatable promoters (such as the ecdysone, Tet-On, altered estrogen receptor (where tamoxifen becomes an agonist), PiP-ON, or metallothionein promoters, adenovirus late promoter, vaccinia virus 7.5K promoter and the like. Promoter/enhancer regions can also be selected to provide tissue specific expression.

The term “operably linked” refers to the relative position of the promoter with respect to a coding sequence such that the promoter controls the transcription of that coding sequence. Those of skill in the art are well aware of how to position a promoter with respect to a coding sequence to create such an operable linkage.

The term “heterologous” when describing the relationship between two or more elements denotes that those elements are not normally found in such a relationship in nature.

The term “targeting sequence” as used herein refers to a DNA sequence that is sufficiently homologous to a portion of the DNA sequence of a target gene to allow homologous recombination to occur within the cell. For the purposes of the present invention, a sequence is sufficiently homologous if it is capable of binding to a target sequence under highly stringent conditions such as, for example, hybridization to filter bound DNA in 0.5 M NaHPO₄, 7% sodium dodecyl sulfate (SDS), 1 mM EDTA at 65° C., and washing in 0.1×SSC/0.1% SDS at 68° C.

A targeting sequence should also, upon transcription and translation in the cell utilized in the methods of this invention, encode a polypeptide that has at least 90% identity to at least a portion of a polypeptide normally produced by that cell and which contains an activatable domain that is activated by the same mechanism as the the activatable domain present in the expression product of the target gene.

The targeting sequences used in this invention may contain intronic sequences that are properly processed by the transcription machinery in the cell. If intornic sequences are included, the targeting sequence will, upon homologous recombination, preferably conserve the endogenous sequence around the intron/exon boundary, and specifically the sequence within nine base pairs of the endogenous splice donor or acceptor sequence.

Preferably targeting sequences useful in this invention comprise 2.5 to 4 kb of genomic sequence derived from the target gene, but may be shorter in length. Typically the desired integration site is chosen to correspond to, or is close to, the start of the portion of the target gene that encodes the activatable domain so as to ensure the expression of an activatable domain following homologous recombination. Generally the targeting sequences used in the present invention are derived from, or corresponds to, the native, or naturally occurring genomic nucleotide sequence in the cell line or tissue being used. Alternatively, the targeting sequence is derived from or corresponds to a nucleotide sequence that is homologous to the target gene. Such homologous sequences are often equivalent to sequences encoding a related target gene in the same cell or in the genome of cells from a related species, genus, order, class, phylum or kingdom.

The first step in designing a targeting sequence is to obtain a copy of the target gene nucleotide sequence (for example, from the NCBI database or any other comparable databases containing genomic information), preferably from the same species from which the cell line to be used for the actual assay is derived. Next, the sequence is analyzed to determine the intron/exon structure and location of splice acceptor and donor sequences. This analysis is typically performed based on homology to known, related genes, and consensus splice acceptor/donor sequences. This can be achieved using publicly available software, such as the Basic Local Alignment Search Tool (“BLAST”).

Typically a targeting sequence is constructed by PCR amplification of a fragment of genomic DNA containing the target gene that is isolated from the target cell line or a related source. Alternatively, the desired fragment of genomic DNA can be isolated from a lambda, P1-phage or BAC library containing the complete genome of the target cell line or any other related source by procedures and protocols known to a person skilled in the art. After obtaining the desired genomic DNA fragment, oligonucleotide primers are designed to produce the desired targeting sequence by PCR.

Typically the sense primer contains one or more restriction enzyme recognition sites allowing for operable linkage of a promoter and, for those embodiments in which a heterologous modulator domain is included in the isolated DNA construct, in frame fusion of that domain to the targeting sequence. The antisense primer preferably contains one or more different restriction enzyme recognition sites allowing for easy cloning of the obtained PCR product into a vector for transfection into the host cells. Such procedures are outlined in more detail in the examples section.

Once the targeting sequence has been obtained, it is typically inserted into a vector already containing or suitable for the further insertion of the heterologous promoter and, in certain embodiments, a heterologous modulator domain. The vector containing all of the desired elements is then introduced to the host cells by any of a number of well-known techniques for getting exogenous DNA into cells. Preferably, the method utilized is electroporation. The procedure utilized to introduce exogenous DNA into cells may be repeated several times, to increase the probability of homologous recombination of the exogenous DNA into the targeted host cell locus.

Suitable vectors that can be used in conjunction with the presently disclosed invention include, but are not limited to, plasmids, and viral vectors. Vectors can include herpes simplex virus vectors, adenovirus vectors, adeno associated virus vectors, retroviral vectors, lentiviral vectors, pseudorabies virus, alpha-herpes virus and the like.

Such vectors may also be engineered to contain selectable markers, that provide for ease in the identification of recipient cells that have at least partially incorporated the DNA construct into the chromatin of the cell. Selection can be based on the use of antibiotic, calorimetric, enzymatic or fluorescent markers to identify cells that have undergone an integration event. Also specifically contemplated for use in the present invention is the use of direct functional selection based on any of the signal transduction detection systems described herein to directly identify cells in which recombination has successfully resulted in the production of an activated peptide that alters the signal from that signal transduction detection system.

The selectable marker gene can be incorporated into the described vectors as a self-contained expression cassette including a selectable marker, promoter for expressing the selectable marker, ribosome binding/translation start site, and polyadenylation sequence. Alternatively, the marker gene can be placed in the vector such that it is expressed from a vector promoter, or it can be engineered to functionally incorporate an independent ribosome entry site (IRES) that facilitates marker expression.

Once the exogenous DNA has been introduced into the population of cells, the cells are incubated under conditions that cause said promoter to drive transcription of an mRNA that encodes said polypeptide and the translation of said mRNA into the encoded polypeptide. When a constitutive promoter is utilized, such conditions are typically standard growth conditions. Accordingly, the conditions are likely to be similar or identical to those conditions employed for growth of the cells following introduction of the exogenous DNA. If an inducible promoter is used, the conditions will include exposing the cells to the appropriate stimulus that induces said promoter. This may include addition of exogenous chemicals to the growth media, change in growth temperature, change in the concentration of one or more media components, change in the pH of the media, etc.

In the next step of this method of the invention, the cells are incubated under conditions that cause activation of said activatable domain of said polypeptide. The selection of such conditions will, of course, be dependent upon the nature of the activatable domain (i.e., what conditions are known to cause activation). In many instances, activation occurs under the same growth conditions utilized to cause expression of the polypeptide. Alternatively, activation may require one or more of addition of exogenous chemicals to the growth media, change in growth temperature, change in the concentration of one or more media components, change in the pH of the media, etc.

Once activation has been allowed to occur, cells that have altered the signal detected from said signal transduction detection system are selected. The method of selecting such cells will be dependent upon the nature of the signal transduction detection system. It will be apparent to those of skill in the art that the generation of a signal from the signal transduction detection system may require the exposure of the cells to an exogenous chemical necessary to detect such signal. As detailed below in the disclosure of exemplary signal transduction detection systems, such exogenous chemicals often include a fluorescent compound that alters either the strength or wavelength of its fluorescence emission, depending upon the state of the signal transduction. Alternatively, the signal transduction causes the production of a naturally fluorescent product, thus eliminating the need to add any exogenous chemicals. It is preferred that the signal transduction detection system produces an optical readout, preferably a fluorescent readout. This is because cells can be easily sorted using a fluorescence activated cell sorter (FACS) on the basis of their fluorescent emission without damaging the cells. Alternative methods include the use of genomic PCR or RT-PCR methods that allow for the direct molecular identification of cells lines that have successfully recombined targeting vectors to the desired genetic loci.

It is also preferred that cells be subjected to two or more rounds of activation and sorting, with periods of cell growth in between sortings. This ensures that the cells have stably incorporated and maintained both the exogenous DNA encoding the polypeptide comprising the activatable domain and the signal transduction detection system. It also allows for selection of those cells that have the highest sensitivity in terms of signal transduction detection system readout to activation. Such cells will allow for more accurate detection of any changes in activation and therefore be more useful in assaying for modulators of the target gene.

In a preferred embodiment, the method of developing a sensor cell for determining the activity of a target gene in said cell is one wherein:
a. said target gene encodes a polypeptide comprising a first modulator domain;
b. said isolated DNA construct further comprises a second modulator domain heterologous to said target gene, wherein said second modulator domain is positioned in said DNA construct relative to said targeting sequence such that following recombination said promoter controls the transcription of an mRNA encoding a polypeptide comprising an activatable domain and said second modulator domain, but lacking said first modulator domain; and
c. upon activation of said activatable domain said modulator domain is capable of altering the signal detected from said signal transduction system.

In this embodiment the homologous recombination event replaces the modulator domain endogenous to the target gene with a modulator domain that is known to be compatible with and can affect the signal output of the signal trasnsduction detection system in the cell. In this manner, one can create a cell useful to assay the activity of a target gene without knowing the endogenous signal transduction pathway. Moreover, utilizing a defined modulator domain allows one to create multiple sensor cells, wherein each cell is able to assay a different target gene activity, starting from a single cell line harboring the same signal transduction detection. This is achieved by utilizing a DNA sequence encoding a modulator domain that is compatible with that signal transduction detection system and fusing it in frame with different targeting sequences, each of which is homologous to a different target gene.

The term “modulator domain,” as used herein, refers to portion of polypeptide that allows the activated form of that polypeptide (i.e., a polypeptide whose activatable domain has been activated) to interact with a signal transduction system. A wide range of modulator domains heterologous to a target gene can be used in the present invention to enable the activation of the polypeptide produced following recombination to be specifically coupled to a defined signal transduction detection system. In general the choice of the modulating domain is dependent on the target gene being modified.

For example, for the modification of target proteins that function as transcription factors, suitable modulating domains include DNA binding domains. In general any heterologous DNA binding domain may be used to replace the DNA binding domain that is normally present in an endogenous target transcription factor. Preferred modulating domains for such targets include those that have well defined DNA recognition motifs that enable the use of a standard signal transduction detection system to be used to detect activation of the target protein in question. Particularly preferred for use in the present invention are DNA binding domains containing one or more zinc finger motifs.

When the target gene encodes a poorly characterized nuclear receptor, DNA binding domains derived from well characterized nuclear receptors or other well characterized transcription factors are particularly useful as heterologous modulator domains. The DNA binding domains from such well-characterized nuclear receptors often well-characterized DNA recognition elements, and defined signal transduction activation pathways. The use of such heterologous modulator domains in the methods of this invention enable the activation of a poorly characterized receptor to be functionally coupled to a defined readout from a signal transduction detection system. Such DNA binding domains include those derived from the nuclear receptors listed in Table 1.

In addition to the use of DNA binding domains from nuclear receptors, heterologous modulator domains useful in this invention may be derived from yeast or bacterially derived transcriptional regulators, co-regulators, repressors and transcription factors comprising zinc finger DNA binding domains. Preferably, such domains are selected from members of the GAL4 and Lex A/Umud super families.

GAL4 (GenBank Accession Number P04386, SEQ ID NO:25) is a positive regulator for the expression of galactose-induced genes such as Gal1, Gal2, Gal7, Gal 10 and Mel1. The DNA binding domain recognizes the 17 base pair sequence (5′-CGGRNNRCYNYNCNCCG-3) (SEQ ID NO:27) in the upstream activating sequences of these genes. GAL4 is structurally and functionally similar to LAC9 of Kluyveromyces lactis.

The DNA binding domain of the yeast Gal4 protein comprises at least the first 74 amino acids of SEQ ID NO:25 [see for example, Keegan et al., Science, 231, pp. 699-704 (1986)]. Preferably for use in the present invention as a modulator domain, the first 96 amino acids of the Gal4 protein (SEQ ID NO:25) are used. Most preferably, the first 147 amino acid residues of yeast Gal4 protein (SEQ ID NO:25) are used.

Full length LexA (GenBank accession number ILEC, (SEQ ID NO:26)) is composed of a structurally distinct N-terminal DNA binding domain and a C-terminal catalytic domain separated by a short hydrophilic hinge region. Members of the LexA family repress a number of genes involved in the response to DNA damage including the RecA and LexA proteins themselves. LexA (SEQ ID NO:26) binds to the 14 bp sequence 5′-CGAACNNNNGTTCG-3′ (SEQ ID NO:28). In the presence of single stranded DNA, RecA interacts with LexA causing an autocatalytic cleavage that disrupts the DNA binding interaction of the protein leading to de-repression of the SOS regulon, the induction of the SOS response, and eventually characteristic error-prone DNA replication and the repair of DNA damage.

For use in the present invention, preferably the first 202 or more amino acids of the LexA protein (SEQ ID NO:26) are used. Most preferably the first 211 amino acid residues of LexA protein (SEQ ID NO:26) are used.

In another embodiment, the invention provides a recombinant sensor cell comprising: a} a signal transduction detection system; and b) a promoter operatively linked to a DNA sequence that encodes a polypeptide comprising an activatable domain, wherein: i) said activatable domain is homologous to all or a portion of a polypeptide encoded by a target gene; ii) said promoter is heterologous to said target gene; and iii) upon expression of said polypeptide and activation of said activatable domain the signal detected from said signal transduction detection system is altered.

In a preferred emodiment, the encoded polypeptide additionally comprises a modulator domain that is heterologous to said target gene, and, upon expression of said polypeptide and activation of said activatable domain, said modulator domain causes the signal detected from said signal transduction system to be altered.

The sensor cells of this invention are preferably created using the methods described above. The preferred signal transduction detection system, promoter, activatable domain, modulator domain and other components of the sensor cells are similar to those described for the method of making a sensor cell.

The sensor cells of this invention are particularly useful for assaying the activity of the target gene and for identifying compounds that are potential modulators of that target gene activity. Accordingly, another embodiment of this invention provides a method of determining if a test compound is a modulator of a target gene or a target gene product comprising the steps of: a) providing a recombinant sensor cell according to this invention; b) incubating said cell in the presence of a test compound under conditions which enable expression of said polypeptide; c) incubating said cell under conditions which enable activation of said activatable domain of said polypeptide; and d) measuring the signal detected from said signal transduction system in said cell. The signal detected is typically compared to the signal detected from the same type of cell that is not exposed to a test compound.

In another embodiment, the invention provides a method of determining the activity of a target gene product comprising the steps of: a) providing a recombinant sensor cell according to this invention; b) incubating said cell under conditions which enable expression of said polypeptide; c) incubating said cell under conditions which enable activation of said activatable domain of said polypeptide; and d) measuring the signal detected from said signal transduction system in said cell. In this embodiment, one would typically compare the singal detected from such a cell with the signal detected from a similar cell wherein the activatable domain of the polypeptide had not been activated.

The preferred signal transduction detection system, promoter, activatable domain, modulator domain (if present) and other components of the sensor cells are similar to those described for the method of making a sensor cell. Even more preferred is that the target gene is a member of the nuclear receptor super family. More preferably, the target gene is an orphan nuclear receptor and the polypeptide additionally comprises a modulator domain that is heterologous to said orphan nuclear receptor.

In yet another embodiment, the invention provides an isolated DNA construct that is useful in the method of making sensor cells of this invention. Such a DNA construct comprises a promoter operatively linked to a DNA sequence which encodes a targeting sequence and a modulator domain, wherein: a) each of said promoter, targeting sequence and modulator domain are heterologous to one another; b) said targeting sequence comprises a region of homology to an endogenous target gene sufficient to promote recombination of said DNA construct; and c) said modulator domain is positioned in said DNA construct with respect to said targeting sequence, such that following said recombination said promoter controls transcription of an mRNA that encodes a polypeptide comprising an activatable domain and said modulator domain.

Signal Transduction Detection Systems

A. Reporter Gene Detection Systems

i) Defined Reporter Gene Constructs

A reporter gene detection system comprises two components—cis-acting and trans-acting elements. Cis-acting elements are non-translated regions of DNA such as promoters, transcription binding sequences, Kozak sequences, enhancers, response elements and 3′ polyadenylation sequences that interact with cellular proteins to carry out transcription and translation of the reporter gene coding sequence. For the purposes of this invention, one or more of these control sequences must be modulatable by the polypeptide whose expression is controlled by the heterologous promoter operatively linked to the targeting sequence following recombination.

The trans-acting sequences encode the reporter gene detection system. This is typically an RNA or protein product that on expression is either directly detectable or detectable through the use of a reagent that interacts with the reporter gene product.

Typically, the reporter gene construct will be cloned into a vector to facilitate the production of plasmid comprising the reporter gene construct. Suitable vectors that can be used in conjunction with the presently disclosed invention include, but are not limited to, non-retroviral plasmids, as well as retroviral vectors such as, herpes simplex virus vectors, adenovirus vectors, adeno-associated virus vectors, retroviral vectors, lentiviral vectors, pseudorabies virus, herpes simplex virus and the like.

A reporter gene construct is generally introduced into a cell using any of a wide variety of known cloning techniques, such as transfection, electroporation, retroviral transduction, and adenoviral transduction.

Selection of those cells that have stably incorporated the reporter gene construct is typically accomplished via induction of reporter gene expression and detection of those cells producing the highest level of reporter gene product as detected by the appropriate functional readout. Additionally, the reporter gene construct may comprise a selectable marker gene, such as an antibiotic resistance gene, a gene encoding a fluorescent product, a gene encoding a colorimetirc product, or a gene encoding a detectable enzyme, thus allowing the detection of the selectable marker to be used to select and enrich for cell clones comprising the reporter gene construct.

The selectable marker gene can be incorporated into the described vectors as a self-contained expression cassette including a selectable marker, promoter for expressing the marker, ribosome binding/translation start site, and polyadenylation sequence. Additionally, the selectable marker can be placed in the vector such that it is expressed from a vector promoter, and can optionally be engineered to functionally incorporate an internal ribosome entry site (IRES) that facilitates selectable marker expression.

The reporter gene construct may exist stably in the cell due to integration into the genome or in the form of a non integrated episomal plasmid (epigenetic). The cell may also harbor the reporter gene construct epigenetic in the form of an Epstein Barr viral construct. The reporter gene construct may be integrated into the genome of the host cell as a single copy or as multiple copies.

Typically, the best reporter cell lines are those that exhibit the best reporter gene induction upon stimulation and the lowest background reporter gene expression. Preferred reporter cell lines exhibit at least a 5-fold increase in reporter gene expression upon stimulation, and preferably at lest a 10-fold increase in reporter gene expression.

The reporter gene detection system may comprise two independent cDNAs or reporter genes that are transcriptionally linked together but give rise to two independent separate proteins. This can be achieved, for example, via the use of an IRES sequence or an A2-self-splicing element. This approach enables the use of one reporter gene to provide for a high throughput optical detection strategy (i.e., FACS), while the other reporter gene can be used for screening. For example, the reporter genes GFP and alkaline phosphatase could be combined via IRES sequence to provide for both an optical detection system (via GFP expression) and a highly sensitivity enzymatic readout from alkaline phosphatase enzymatic activity. Another example contemplated by the invention is a construct encoding luciferase and β-lactamase under control of the P-lactamase promoter, wherein the luciferase and β-lactamase coding sequences are coupled via an IRES or A2 self-splicing element.

ii) Random Integration Gene Trap

Another potential source of reporter gene constructs are those created using random insertion of a reporter gene into the host cell genome (“gene trap”). This approach is described in U.S. Pat. No. 5,928,888, the disclosure of which is herein incorporated by reference. Cell lines created by this methodology possess a reporter gene under the control of one of the cells native promoter. Thus, stimuli that normally turn on the gene controlled by that native promoter, now express the reporter gene. In practice, such reporter genes may be under the control of a promoter that is modulated by other proteins in the same biochemical pathway. Thus, such cells effectively contain a reporter gene construct that functions as a signal transduction detection system for those other proteins in the biochemical pathway. For example, applicants have used the gene trap approach to create a cell line where the reporter gene (6-lactamase) is under control of the EGR-3 control sequences. That cell line was identified by its ability to respond to T-cell receptor activation by phytohemagglutinin (PHA), which directly induces EGR-3 expression. However, applicants have also found that the same cell line also responds through a number of G protein-coupled receptors (GPCR)s. Thus, this cell line could be used to in the methods of the present invention to assay modulators of GPCR.

iii) Reporter Genes

The choice of reporter gene suitable for use in the present invention includes any enzyme capable of catalyzing the creation of a detectable product. Specific examples include, without limitation, alkaline phosphatase, β-galactosidase, chloramphenicol acetyltransferase, β-glucuronidase, peroxidase, β-lactamase, catalytic antibodies, luciferases and other bioluminescent proteins. It is to be understood that for those reporter genes that do not readily lead to, or generate, a live cell assay that produces an optical signal, it preferred to include at least one additional signal transduction detection system that does generate an optical signal compatible with high throughput functional analysis.

This is so because not all cells that are transformed with a reporter gene DNA construct will stably take up and express the reporter gene at sufficient levels to be useful in the methods of this invention. One must separate those cells that stably express the reporter gene at sufficiently high levels from those cells that do not, without killing the cells. This is best achieved through cell sorting based upon optical detection (e.g., FACS). Such duel detection systems can include any one of the reporter genes disclosed herein coupled with, for example, a voltage sensor, a biosensor, an ion sensitive dye or a reporter gene that does give rise to an optical signal, such as, for example, a naturally fluorescent protein, β-lactamase, β-galactosidase etc.

Alkaline phosphatase, including human placental and calf intestinal alkaline phosphatase (for example, GenBank Accession #U89937), can be measured using colorimetric, fluorescent and chemiluminescent substrates [Berger, J., et al. (1988) Gene, 66, pp. 1-10; Kain, S. R., Methods. Mol. Biol., 63, pp. 49-60 (1997)]. Alkaline phosphatase is widely used in transcriptional assays, and is typically by measuring secreted alkaline phosphatase (SeAP).

β-galactosidase (β-Gal), the gene product of the bacterial gene LacZ, is also widely used as a reporter gene for transcriptional analysis and may be assayed via histochemical, fluorescent or chemiluminescent substrates, either within intact, or permeabilized cells [U.S. Pat. No. 5,070,012; and Bronstein, I., et al., J. Chemilum. Biolum., 4, pp. 99-111 (1989)].

β-glucuronidase (GUS) is widely used for transcriptional analysis in higher plants and may also be assayed using a variety of histochemical and fluorescent substrates [U.S. Pat. No. 5,599,670].

Chloramphenicol acetyltransferase (CAT), encoded by the bacterial Tn9 gene, is widely used for transcriptional assays and is traditionally measured using a radioisotopic assay in cell extracts [Gorman et al., Mol. Cell. Biol., 2, pp. 1044-51 (1982].

Catalytic antibodies are also amenable for use as reporter genes, if the reaction catalyzed by the antibody results in the formation of a detectable product. Useful examples include the aldolase specific antibodies 38C2 and 33F12 that catalyze the synthesis of novel fluorogenic retro-aldol reactions [List et al., Proc. Natl. Acad. Sci. USA, 95, pp. 15351-15355 (1998)]. Typical antibody substrates are cell permeant, nonpolar, organic molecules that are not substrates for the natural enzymes and are thus good markers of enzyme activity.

A large number of β-lactamases have been isolated and characterized, all of which are suitable for use in accordance with the present invention [for review see, Ambler, R. P., Phil. Trans. R. Soc. Lond., 289, pp. 321-331 (1980)]. The coding regions and encoded proteins of a exemplary β-lactamases, employed in the methods described herein include SEQ ID NOS:1 through 10. Nucleic acids encoding proteins with β-lactamase activity can be obtained by methods known in the art, for example, by polymerase chain reaction of cDNA using primers based on a DNA sequence in SEQ ID NOS: 1, 3, 5, 7 or 9.

Preferably, β-lactamase polynucleotides utilized in the present invention encode an intracellular form of a protein with beta-lactamase activity that lacks a functional signal sequence. This provides the advantage of trapping the normally secreted β-lactamase protein within the cell, which enhances the signal to noise ratio of the signal associated with β-lactamase activity, and enables the individual cells to be subjected to FACS upon the addition to the cells of a membrane permeant fluorescent substrate for the enzyme. For example, in any of the polypeptides of SEQ ID NOS: 2, 4, 6, 8 or 10, the signal sequence has been replaced with the amino acids Met-Ser. Accordingly, upon expression, β-lactamase activity remains within the cell. For expression in mammalian cells it may be preferable to use β-lactamase polynucleotides with nucleotide sequences preferred by mammalian cells. In some applications secreted forms of β-lactamase can be used with the methods described herein.

A variety of colorimetric and fluorescent substrates of β-lactamase are available. Fluorescent substrates include those capable of changes, either individually or in combination, of total fluorescence, excitation or emission spectra or fluorescence resonance energy transfer (FRET), for example those described in U.S. Pat. Nos. 5,741,657 and 5,955,604. Any membrane permanent β-lactamase substrate capable of being measured inside the cell after cleavage can be used in the methods and compositions of the invention. Membrane permanent β-lactamase substrates do not require permeablizing eukaryotic cells either by hypotonic shock or by electroporation. Generally, such non-specific pore forming methods are not desirable to use in eukaryotic cells because such methods injure the cells, thereby decreasing viability and introducing additional variables into the screening assay.

Preferably, the membrane permeant β-lactamase substrates are transformed in the cell into a β-lactamase substrate of reduced membrane permeability or that is membrane impermeant. Transformation inside the cell can occur via membrane-associated esterases or intracellular metabolites or organic molecules (e.g. sulfhydryl groups). Preferred substrates for measuring β-lactamase include CCF2/AM and CCF4/AM, and their free acid forms. The substrates CCF2/AM and CCF4/AM may be used inter-changeably for the purposes described herein.

Preferred bioluminescent proteins useful as reporter genes in the methods of this invention include firefly, bacterial or click beetle luciferases, aequorins and other photoproteins, (for example as described in U.S. Pat. Nos. 5,221,623, 5,683,888, 5,674,713, 5,650,289 and 5,843,746). Particularly preferred are bioluminescent proteins isolated from the ostracod Cypridina (or Vargula) hilgendorfii [Johnson and Shimomura, Methods Enzymol., 57, pp. 331-364 (1978); and Thompson, Nagata & Tsuji, Proc. Natl. Acad. Sci. USA, 86, pp. 6567-6571 (1989)].

Beyond the availability of bioluminescent proteins (luciferases) isolated directly from the light organs of beetles, cDNAs encoding luciferases of several beetle species are available. These include the luciferase of P. pyralis (firefly), the four luciferase isozymes of P. plagiophthalamus (click beetle), the luciferase of L. cruciata (firefly) and the luciferase of L. lateralis [deWet et al., Molec. Cell. Biol., 7, pp. 725-737 (1987); Masuda et al., Gene, 77, pp. 265-270 (1989); Wood et al., Science, 244, pp. 700-702 (1989); and European Patent Application Publication No. 0 353 464). Further, the cDNAs encoding luciferases of any other beetle species, which make bioluminescent proteins, are readily obtainable by the skilled using known techniques [de Wet et al., Meth. Enzymol., 133, pp. 3-14 (1986); Wood et al., supra].

Most firefly and click beetle luciferases are ATP-magnesium-dependent and require oxygen for light production. Typically light emission from these enzymes exhibits a burst in intensity followed by a rapid decrease in the first few seconds, followed by a lower sustained light emission. Relatively sustained light output at high rates can be accomplished in these systems by inclusion of coenzyme A, dithiothreitol and other reducing agents that reduce product inhibition and slow inactivation of the luciferase from byproducts of the light generating reaction (see U.S. Pat. Nos. 5,641,641 and 5,650,289, the disclosures of which are herein incorporated by reference). Such stable light emitting systems are preferred for use in the present invention.

Particularly preferred bioluminescent proteins are those derived from the ostracod Cypridina (or Vargula) hilgendorfii. The Cypridina luciferase (GenBank Accession No. U89490) uses no cofactors other than water and oxygen, and its luminescent reaction proceeds optimally at pH 7.2 and physiological salt concentrations, (Shimomura, O. et al, J. Cell. Comp. Physiol., 58, pp. 113-124 (1961)). By comparison, firefly luciferase has optimal activity at low ionic strength, alkaline pH and reducing conditions, that are typically quite different to those usually found within mammalian cells. The Cypridina luciferase produces a specific photon flux exceeding that of the optimized firefly system by a factor of at least 50 [Miesenbock and Rothman, Proc. Natl. Acad. Sci. USA, 94, pp. 3402-3407 (1997)].

Another preferred class of reporter genes useful in this invention is naturally fluorescent proteins such as the Green Fluorescent Protein (GFP) of Aequorea victoria [Tsien, R. Y. Ann. Rev. Biochem., 67, pp. 509-44 (1998)]. The use of naturally fluorescent proteins as reporter genes avoids the use of additional co-factors or fluorophores. Thus, such proteins provide the ability to monitor activities within defined cell populations, tissues, or in an entire transgenic organism. For example, by using cell type specific promoters and subcellular targeting motifs, it is possible to selectively target the naturally fluorescent protein to a discrete location within the cell to enable highly spatially defined measurements.

Naturally fluorescent proteins useful in the present invention have been isolated and cloned from a number of marine species including the sea pansies Renilla renifonnis, R. kollikeri and R. mullerei and from the sea pens Ptilosarcus, Stylatula and Acanthoptilum, as well as from the Pacific Northwest jellyfish, Aequorea victoria [Szent-Gyorgyi et al. (SPIE conference 1999); D. C. Prasher et al., Gene, 111, pp. 229-233 (1992)] and several species of coral [Matz et al, Nature Biotechnology, 17, pp. 969-973 (1999)]. These proteins are capable of forming a highly fluorescent, intrinsic chromophores through the cyclization and oxidation of internal amino acids within the protein that can be spectrally resolved from weakly fluorescent amino acids such as tryptophan and tyrosine.

Naturally fluorescent proteins have also been observed in other organisms, although in most cases these require the addition of some exogenous factor to enable fluorescence development. These incude the yellow fluorescent protein from Vibrio fischeri strain Y-1 [T. O. Baldwin et al., Biochemistry, 29, pp. 5509-15 (1990)], which requires flavins as fluorescent co-factors; Peridinin-chlorophyll a binding protein from the dinoflagellate Symbiodinium sp. [B. J. Morris et al., Plant Molecular Biology, 24, pp. 673-77 (1994), which fluoresces in the red spectrum; and phycobiliproteins from marine cyanobacteria such as Synechococcus, e.g., phycoerythrin and phycocyanin [S. M. Wilbanks et al., J. Biol. Chem., 268, pp. 1226-35 (1993), which require phycobilins as fluorescent co-factors, the insertion of which into the proteins requires auxiliary enzymes.

A variety of mutants of the GFP from Aequorea victoria have been created that have distinct spectral properties, improved brightness and enhanced expression and folding in mammalian cells compared to the native GFP, (e.g., SEQ ID NOS:11 and 12; Table 2) [Green Fluorescent Proteins, Chapter 2, pp. 19-47, Sullivan and Kay, eds., Academic Press; U.S. Pat. Nos. 5,625,048; 5,777,079; and 5,804,387]. In many cases these functional engineered fluorescent proteins have superior spectral properties to wild-type Aequorea GFP and are preferred for use as reporters in the present invention.

TABLE 2
Aequorea Fluorescent Proteins

| | | Quantum | | | |
|---|---|---|---|---|---|
| | | Yield (Φ) & | Excitation & | Relative | maximum |
| | Common | Molar | Emission Maxima | Fluorescence | fluoresecence |
| Mutations | Name | Extinction (ε) | (nm) | At 37° C. (%) | at pH 6 |
| S65T type | | | | | |
| S65T, S72A, | Emerald | Φ = 0.68 | 487 | 100 | 91 |
| N149K, | | ε = 57,500 | 509 | | |
| M15T, | | | | | |
| I167T | | | | | |
| F64L, S65T, | | Φ = 0.58 | 488 | 54 | 43 |
| V163A | | ε = 42,000 | 511 | | |
| F64L, S65T | EGFP | Φ = 0.60 | 488 | 20 | 57 |
| | | ε = 55,900 | 507 | | |
| S65T | | Φ = 0.64 | 489 | 12 | 56 |
| | | ε = 52,000 | 511 | | |
| Y66H type | | | | | |
| F64L, Y66H, | P4-3E | Φ = 0.27 | 384 | 100 | N.D. |
| Y145F, | | ε = 22,000 | 448 | | |
| V163A | | | | | |
| F64L, Y66H, | | Φ = 0.26 | 383 | 82 | 57 |
| Y145F | | ε = 26,300 | 447 | | |
| Y66H, | P4-3 | Φ = 0.3 | 382 | 51 | 64 |
| Y145F | | ε = 22,300 | 446 | | |
| Y66H | BFP | Φ = 0.24 | 384 | 15 | 59 |
| | | ε = 21,000 | 448 | | |
| Y66W type | | | | | |
| S65A, | W1C | Φ = 0.39 | 435 | 100 | 82 |
| Y66W, | | ε = 21,200 | 495 | | |
| S72A, | | | | | |
| N146I, | | | | | |
| M153T, | | | | | |
| V163A | | | | | |
| F64L, S65T, | W1B, CFP | Φ = 0.4 | 434 452 | 80 | 71 |
| Y66W, | | ε = 32,500 | 476 (505) | | |
| N146I, | | | | | |
| M153T, | | | | | |
| V163A | | | | | |
| Y66W, | hW7 | Φ = 0.42 | 434 452 | 61 | 88 |
| N146I, | | ε = 23,900 | 476 (505) | | |
| M153T, | | | | | |
| V163A | | | | | |
| Y66W | | | 436 | N.D. | N.D. |
| | | | 485 | | |
| T203Y type | | | | | |
| S65G, S72A, | Topaz, YFP | Φ = 0.60 | 514 | 100 | 14 |
| K79R, | | ε = 94,500 | 527 | | |
| T203Y | | | | | |
| S65G, V68L, | 10C | Φ = 0.61 | 514 | 58 | 21 |
| S72A, | | ε = 83,400 | 527 | | |
| T203Y | | | | | |
| S65G, V68L, | h10C+ | Φ = 0.71 | 516 | 50 | 54 |
| Q69K, | | ε = 62,000 | 529 | | |
| S72A, | | | | | |
| T203Y | | | | | |
| S65G, S72A, | | Φ = 0.78 | 508 | 12 | 30 |
| T203H | | ε = 48,500 | 518 | | |
| S65G, S72A | | Φ = 0.70 | 512 | 6 | 28 |
| T203F | | ε = 65,500 | 522 | | |
| T203I type | | | | | |
| T203I, | Sapphire | Φ = 0.64 | 395 | 100 | 90 |
| S72A, | | ε = 29,000 | 511 | | |
| Y145F | | | | | |
| T203I | H9 | Φ = 0.6 | 395 | 13 | 80 |
| T202F | | ε = 20,000 | 511 | | |

Non Aequorea, naturally fluorescent proteins, for example Anthozoan fluorescent proteins, and functional engineered homologs, thereof, are also suitable for use in the present invention including those shown in Table 3 below.

TABLE 3
Anthozoa Fluorescent Proteins

| | | Quantum | | | |
|---|---|---|---|---|---|
| | | Yield (Φ) & | | | |
| | | Molar | Excitation & | Relative | |
| Species | Protein Name | Extinction (ε) | Emission Max | Brightness | SEQ ID NO: |
| Anemonia | amFP486 | Φ = 0.24 | 458 | 0.43 | SEQ ID NOS: 13 |
| majano | | ε = 40,000 | 486 | | and 14 |
| Zoanthus sp | zFP506 | ε = 35,600 | 496, 506 | 1.02 | SEQ ID NOS: 15 |
| | | | | | and 16 |
| | zFP538 | Φ = 0.42 | 528, 538 | 0.38 | SEQ ID NOS: 17 |
| | | | | | and 18 |
| Discosoma | dsFP483 | Φ = 0.46 | 443 | 0.5 | SEQ ID NOS: 19 |
| striata | | ε = 23,900 | 483 | | and 20 |
| Discosoma sp | drFP583 | Φ = 0.23 | 558 | 0.24 | SEQ ID NOS: 21 |
| “red” | | ε = 22,500 | 583 | | and 22 |
| Clavularia sp | CFP484 | Φ = 0.48 | 456 | 0.77 | SEQ ID NOS: 23 |
| | | ε = 35,300 | 484 | | and 24 |

Methods of Measurement and Screening

Methods of performing screening assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y. L., San Diego: Academic Press (1989), pp. 219-243; Turro, N. J., Modern Molecular Photochemistry, Menlo Park: Benjamin/Cummings Publishing Col, Inc. (1978), pp. 296-361.

Fluorescence in a sample can be measured using a fluorimeter, a fluorescent microscope or a fluorescent plate reader. In general, all of these systems have an excitation light source that can be manipulated to create a light source with a defined wavelength maxima and band width that passes through excitation optics to excite the sample.

Typically, the excitation wavelength is designed to selectively excite the fluorescent sample within its excitation or absorption spectrum. For most FRET based assays the excitation wavelength is usually selected to enable efficient excitation of the donor while minimizing direct excitation of the acceptor. The sample, if fluorescent, emits radiation that has a wavelength different from the excitation wavelength. Optics then collects the emission from the sample, and directs it to one or more detectors, such as photomultiplier tubes or CCD cameras. Preferably the detector will include a filter to select specific wavelengths of light to monitor.

Suitable instrumentation for screening, include for example, fluorescence microplate readers include the CytoFluor™ 4000 available from PerSeptive Biosystems. Suitable instrumentation for high throughput miniaturized screening includes instruments capable of reading 1536 and 3456 multiwell plates. Such instruments enable miniaturized high throughput screening in volumes or about 1 to 5 μL. Suitable instrumentation for flow cytometry includes the FACS Vantage SE™ and FACS Vantage™ flow cytometers from Becton Dickinson (BD).

For FACS one can use two techniques for analyzing and sorting cells based on the β-lactamase/CCF2 reporter system. One technique uses log scale fluorescence analysis and fluorescence compensation. On some flow cytometers, this technique has the advantage that pulse processing is not required, and the instrument's dead time is minimized, thereby enabling higher sort throughput. The other technique uses linear scale fluorescence analysis, no fluorescence compensation and real-time ratio analysis, in a manner analogous to that typically used for Indo-1 (19). Either violet (typically 407 nm or 413 nm from a krypton laser), or UV excitation can be used for the beta lactamase substrates CCF2 or CCF4.

In general, violet excitation (413 nm) is preferred with CCF2 and CCF4 because this is closer to the excitation maximum (409 nm) of the donor molecule (coumarin) in CCF2, and because a great deal of avoiding UV excitation reduces cellular autofluorescence in the blue channel.

Using log scale fluorescence intensities and fluorescence compensation allows for two-dimensional visualization of the fluorescence distribution of a population, and has potential throughput advantages. In practice, 10% fluorescence compensation blue from green and the recropical is typically applied, due to the spectral overlap of the cleaved and un-cleaved forms of CCF2/AM OR CCF4/AM.

B. Voltage Sensors

Another class of signal transduction detection systems suitable for use with the present invention is high throughput assay systems capable of detecting transmembrane potential changes. These methods include, for example, automated patch clamping [(Hamill et al, Pfluegers Arch., 391, pp. 85-100 (1981)], FRET based voltage sensors, electrochromic transmembrane potential dyes [Cohen et al., Ann. Rev. Neurosci., 1, pp. 171-82 (1978)1, transmembrane potential redistribution dyes (Loew, L. M., Potentiometric membrane dyes, in: Fluorescent and Luminescent Probes for Biological Activity, ed. Mason, W. T., San Diego: Academic Press(1993), pp. 150-160), extracellular electrodes [Thomas et al., Exp. Cell. Res., 74, pp. 61-66 (1972)], field effect transistors [Fromherz et al., Science, 252, pp. 1290-1293 (1991], radioactive flux assays, ion sensitive fluorescent or luminescent dyes, ion sensitive fluorescent or luminescent proteins, the expression of endogenous proteins or the use of reporter genes or molecules.

Preferred methods include the use of optical readouts of transmembrane potential, or ion channel conductance. Such methods include the use of transmembrane potential or ion sensitive dyes, or molecules that typically exhibit a change in their fluorescent or luminescent characteristics as a result of changes in ion channel conductance or transmembrane potential.

A preferred optical method of analysis for use with the present invention has been described in U.S. Pat. No. 5,661,035, which is herein incorporated by reference. This approach typically comprises two reagents that undergo energy transfer to provide a ratiometric fluorescent readout that is dependent upon the transmembrane potential. Typically the approach uses a voltage sensing lipophilic dye and a voltage insensitive fluorophore associated with a cell membrane [Gonzalez et al., Drug Discovery Today, 4, pp. 431-439 (1999)].

In one embodiment, two dye molecules—a coumarin-linked phospholipid (CC2-DMPE) and an oxonol dye such as bis-(1,2-dibutylbarbituric acid) trimethine oxonol [DiSBAC₄ (3)]—are loaded into the plasma membrane of cells. CC2-DMPE partitions into the outer leaflet of the plasma membrane where it acts as a fixed FRET donor to the mobile, voltage sensitive oxonol acceptor. Cells with relatively negative potentials inside will push the negatively charged oxonol to the outer leaflet of the plasma membrane, resulting in efficient FRET (i.e. quenching of the coumarin donor and excitation of the oxonol acceptor). Depolarization results in rapid translocation of the oxonol to the inner surface of the plasma membrane, decreasing FRET. Because FRET can only occur over distances of less than 100 Å, excitation of the coumarin results in specific monitoring of oxonol movements within the plasma membrane.

The response times for these assays is readily altered by increasing or decreasing the hydrophobicity of the oxonol. For example, the more hydrophobic dibutyl oxonol DiSBAC₄(3) has a time constant of approximately 10 ms, significantly faster than the less hydrophobic diethyl oxonol DiSBAC₂(3).

Loading of the dyes is typically achieved at room temperature prior to the start of transmembrane potential measurements. Typically cells are loaded sequentially with the coumarin lipid followed by the oxonol. Typical loading concentrations for coumarin lipids range from about 4 to 15 μM (final concentration) and staining solutions are typically prepared in Hanks Balanced salt solution with 10 mM HEPES, 2 g/L glucose and about 0.02% Pluronic-127 at a pH of around 7.2 to 7.4. Loading is usually acceptable after about 30 minutes incubation, after which excess dye may be removed if desired. Oxonol dyes are typically loaded at a concentration between 2 and 10 μM for 25 minutes at room temperature, the more hydrophobic DiSBAC₄(3) is usually loaded in the presence of 2-3 μM Pluronic-127. Optimal loading concentrations vary between cell types and can be empirically determined by routine experimentation. Typically such optimization experiments are conducted by systematically titrating the concentrations of the first reagent, and then for each concentration tested, titrating the concentration of the second reagent. In this way it is possible to obtain both the optimal loading concentrations for each reagent, and the optimal relative ratio to achieve a maximal signal to noise ratio.

In some cases it may be preferred to add or load one or more of the FRET reagents with one or more light absorbing substances in order to reduce undesired light emission, as for example described in commonly owned U.S. Pat. Nos. 6,200,762; 6,214,563; and 6,221,612, the disclosures of which are herein incorporated by reference.

FRET based voltage sensors may also be derived from the use of other membrane-targeted fluorophores in conjunction with a mobile hydrophobic donor or acceptor. Other such compositions are disclosed, for example, in U.S. Pat. No. 6,342,379.

Methods of Measurement and Screening

Suitable instrumentation for measuring transmembrane potential changes via optical methods includes microscopes, multiwell plate readers and other instrumentation that is capable of rapid, sensitive ratiometric fluorescence detection. A preferred instrument of this type is described in U.S. Pat. No. 6,349,160. This instrument (the Voltage/Ion Probe Reader or VIPR™) is an integrated liquid handler and kinetic fluorescence reader for 96-well and greater multiwell plates. The VIPR™ reader integrates an eight channel liquid handler, a multiwell positioning stage and a fiber-optic illumination and detection system. The system is designed to measure fluorescence from a column of eight wells simultaneously before, during and after the introduction of liquid sample obtained from another microtiter plate or trough. The VIPR™ reader excites and detects emission signals from the bottom of a multiwell plate by employing eight trifurcated optical bundles (one bundle for each well). One leg of the trifurcated fiber is used as an excitation source, the other two legs of the trifurcated fiber being used to detect fluorescence emission. A ball lens on the end of the fiber increases the efficiency of light excitation and collection. The bifurcated emission fibers allow the reader to detect two emission signals simultaneously and are compatible with rapid signals generated by the FRET-based voltage dyes. Photomultiplier tubes then detect emission fluorescence, enabling sub-second emission ratio detection.

C. Bio-Sensors for Measuring Post Translational Modifications.

Another class of signal transduction detection systems are those based on naturally fluorescent or luminescent proteins that can be used to measure post-translational and other activities, including without limitation proteolysis, phosphorylation, dephosphorylation, glycosylation, methylation, sulfation, prenylation, redox, disulfide bond formation, intracellular ion concentrations and ADP-ribsoylation within living cells. Examples of such biosensors include without limitation, those disclosed in the following publications: Ting A. Y. et al., Proc Natl Acad Sci U.S.A., 98, pp. 15003-8 (2001); Zhang J. et al., Proc Natl Acad Sci U.S.A., 98, pp. 14997-5002 (2001); Chan F. K. et al., Cytometry, 44, pp. 361-8 (2001); Burdette S. C. et al., J. Am. Chem. Soc., 123, pp. 7831-41 (2001); Honda A. et al., Proc. Natl. Acad. Sci. U.S.A., 98, pp. 2437-42 (2001); Miyawaki A et al., Methods Enzymol., 327, pp. 472-500 (2000); Baird G. S. et al., Proc. Natl. Acad. Sci. U.S.A., 96, pp. 11241-6 (1999); Wachter R. M. et al., Curr. Biol., 9, pp. R628-9 (1999); Miyawaki A. et al., Proc. Natl. Acad. Sci. U.S.A., 96, pp. 2135-40 (1999); Jayaraman S. et al., J. Biol. Chem., 275, pp. 6047-50 (2000); U.S. Pat. Nos. 5,981,200; 5,925,558; 6,054,321; 6,077,707; 6,197,928; 5,998,204; 6,054,271; 6,008,378; 5,932,474 and 6,410,255, all of which are herein incorporated by reference.

Methods of Measurement and Screening

Typically the same or similar methods of analysis and screening can be applied to the measurement of fluorescent biosensors as previously described for other fluorophores. For example standard fluorimeters and fluorescent plate readers such as the CytoFluor 4000 can be used to measure fluorescence from the biosensors described above.

D. Ion Sensitive Dyes

i) Calcium Indicators

Fura-2 and indo-1 are UV light-excitable, ratiometric Ca²⁺ indicators that are generally considered to be interchangeable in most experiments. Fura-2 has become the dye of choice for ratio-imaging microscopy, in which it is more practical to change excitation wavelengths than emission wavelengths. Upon binding Ca²⁺, fura-2 exhibits an absorption shift that can be observed by scanning the excitation spectrum between 300 and 400 nm, while monitoring the emission at ˜510 nm. In contrast, indo-1 is the preferred dye for flow cytometry, where it is more practical to use a single laser for excitation—usually the 351-364 nm spectral lines of the argon-ion laser—and monitor two emissions. The emission maximum of indo-1 shifts from ˜475 nm in Ca²⁺-free medium to ˜400 nm when the dye is saturated with Ca²⁺. Modern two-photon excitation imaging techniques used with fura-2 and indo-1 avoid the deleterious effects of conventional ultraviolet illumination on living specimens. Indo-1 may be less subject to compartmentalization than fura-2, whereas fura-2 is more resistant to photobleaching than indo-1. Both fura-2 and indo-1 exhibit Kd values that are close to typical basal Ca²+ levels in mammalian cells (˜100 nM), and display high selectivity for Ca²+ binding relative to Mg²⁺. Nevertheless, Ca²+ binding is discernibly perturbed by physiological levels of Mg²⁺; the Kd for Ca²⁺ of fura-2 is ˜135 nM in Mg²⁺-free Ca²⁺ buffers and ˜224 nM in the presence of 1 mM Mg²⁺. Fura-2 and indo-1 also exhibit high affinities for other divalent cations such as Zn²⁺ and Mn²⁺.

Calcium concentrations above 1 μM produce almost complete binding saturation of fura-2 but very low fractional saturation of the low-affinity fura analog mag-fura-2. To bridge this gap in the Ca²⁺ measurement range of fura-type indicators, three additional ratiometric Ca²⁺ indicators may be used—fura-4F, fura-5F, and fura-6F—and their corresponding membrane-permeant AM ester derivatives.

Attachment of a single electron-withdrawing fluorine substituent at different positions on the BAPTA chelator moiety of fura-2 results in an increase of the Kd value to ˜770 nM, ˜400 nM and 5.3 μM for fura-4F, fura-5F and fura-6F, respectively. Except for the change in the Ca²⁺ concentration response range the Ca²⁺-dependent spectral shifts produced by fura-4F, fura-5F and fura-6F are essentially identical to those of fura-2 and the probes use the same optical filter sets. Fura-FF is a difluorinated derivative of fura-2 with a Kd value of ˜5.5 μM. Fura-FF has high selectivity for Ca²⁺, a wide dynamic range and low pH sensitivity, making it an optimal low-affinity Ca²⁺ indicator for most imaging applications. Although its spectroscopic characteristics are very similar to those of mag-fura-2, fura-FF has negligible Mg²⁺ sensitivity, making Ca²⁺ detection less susceptible to interference. These properties have made fura-FF particularly useful for spatial and functional characterization of intracellular Ca²⁺ stores and for tracking Ca²⁺ oscillations driven by the inositol 1,4,5-triphosphate receptor. The low-affinity indicator fura-FF could detect NMDA- and kainate-induced neuronal Ca²⁺ fluxes that were not detectable with the higher-affinity indicator fura-2. Fura-FF has also been used in combination with fura-2 and mag-fura-5 to compare the actions of Sr²⁺ and Ca²⁺ as mediators of synaptic transmission.

Indo-5F is an analog of indo-1 designed for measuring Ca²⁺ concentrations above 1 μM. Like indo-1, indo-5F exhibits Ca²⁺-dependent dual-emission, making it suitable for ratiometric detection by flow cytometry.

ii) Low-Affinity Calcium Indicators

The coumarin benzothiazole-based Ca²⁺ indicator BTC exhibits a shift in excitation maximum from about 480 nm to 400 nm upon binding Ca²⁺, permitting ratiometric measurements that are essentially independent of uneven dye loading, cell thickness, photobleaching and dye leakage. Its high selectivity and moderate affinity for Ca²⁺ (Kd ˜7 μM) allows accurate quantitation of high intracellular Ca²⁺ levels that are underestimated by fura-2 measurements.

Furthermore, because BTC is excited at longer wavelengths than the ratioable fura-2 and indo-1 indicators, cellular photodamage and autofluorescence may be less of a problem. When loaded into neurons as its AM ester, BTC exhibits little compartmentalization. However, prolonged excitation appears to cause conversion of the indicator to a calcium-insensitive form. BTC has been employed in investigations of Ca²⁺-dependent exocytosis in pancreatic cells, CHO fibroblasts and phaeochromocytoma cells.

Neuronal Ca²⁺ transients detected by the low-affinity Ca²⁺ indicators BTC and mag-fura-2 are significantly more rapid than those reported by the higher-affinity indicators fura-2 and Calcium Green-2.

BTC may also be useful as an indicator for Zn²⁺. Mag-fura-2 (also called furaptra), mag-fura-5 and mag-indo-1 (Fluorescent Magnesium Indicators) were originally designed to report intracellular Mg²⁺ levels however, these indicators actually have much higher affinity for Ca²⁺ than for Mg²⁺. Although Ca²⁺ binding by these indicators may complicate analysis when they are employed to measure intracellular Mg²⁺, their increased effective range and improved linearity for Ca²⁺ measurements has been exploited for measuring intracellular Ca²⁺ levels between 1 μM and 100 μM.

The spectral shifts of mag-fura-2, mag-fura-5 and mag-indo-1 are very similar to those of fura-2 and indo-1 but occur at higher Ca²⁺ concentrations. Because the off-rates for Ca²⁺ binding of these indicators are faster than those of fura-2 and indo-1, these dyes have been used to monitor action potentials in skeletal muscle and nerve terminals with little or no kinetic delay. The moderate Ca²⁺ affinity of mag-fura-2 and the tendency of its acetoxymethyl (AM) ester to accumulate in subcellular compartments have proven useful for in situ monitoring of inositol 1,4,5-triphosphate-sensitive Ca²⁺ stores. Mag-fura-2 has also been employed to follow Ca²⁺ transients in presynaptic nerve terminals, gastric epithelial cells and cultured myocytes. Imaging of mag-fura-2 using a single excitation wavelength (420 nm) is reported to improve the detection of high-level Ca²⁺ transients in various cells, including Purkinje neurons and frog muscle. Mag-indo-1 has been used to detect gonadotropin-releasing hormone-induced Ca²⁺ oscillations in gonadotropes and to investigate the role of Ca²⁺/K+ exchange in intracellular Ca²⁺ storage and release processes. Measurements of Ca²⁺ currents in presynaptic boutons and granule cell parallel fibers with mag-fura-5 and Magnesium Green indicators were shown to be superior to those made using fura-2 (data not shown). Mag-fura-2, mag-fura-5 and mag-indo-1 are available as cell-impermeant potassium salts or as cell-permeant AM esters.

iii) Visible Light Excitatable Ca²⁺ Indicators

Visible light-excitable indicators offer several advantages over UV light- excitable indicators, including i) efficient excitation with most laser-based instrumentation, including confocal laser-scanning microscopes and flow cytometers, ii) reduced interference from sample autofluorescence, iii) less cellular photodamage and light scatter, iv) stronger absorption by the dyes, which may permit the use of lower dye concentrations and therefore lower phototoxicity to live cells, v) compatibility with photoactivatable (“caged”) probes and other UV light-absorbing reagents, increasing options for multiparameter measurements, and vi) large Ca²⁺-dependent fluorescence intensity increases, resulting in sensitive detection of Ca²⁺ transients. Several of these indicators are described in detail below.

Fluo-3

The Ca²⁺ indicator fluo-3 was developed for use with visible-light excitation sources in flow cytometry and confocal laser-scanning microscopy. More recently, fluo-3 imaging has been extended to include two-photon excitation techniques. Fluo-3 imaging has revealed the spatial dynamics of many elementary processes in Ca²⁺ signaling. More recently, fluo-3 has been extensively used in cell-based high-throughput screening assays for drug discovery. Fluo-3 is essentially nonfluorescent unless bound to Ca²⁺ and exhibits a quantum yield at saturating Ca²⁺ of ˜0.14. The intact acetoxymethyl (AM) ester derivative of fluo-3 is also nonfluorescent, unlike the AM esters of fura-2 and indo-1.

The green-fluorescent emission (˜525 nm) of Ca²⁺-bound fluo-3 is conventionally detected using optical filter sets designed for fluorescein (FITC). The fluorescence output of fluo-3—the product of the molar absorptivity and the fluorescence quantum yield—may also vary significantly in different cellular environments. Fluo-3 lacks a significant shift in emission or excitation wavelength upon binding to Ca²⁺, which precludes the use of ratiometric measurements.

Simultaneous loading of cells with fluo-3 and Fura Red (see below), which exhibit reciprocal shifts in fluorescence intensity upon binding Ca²⁺, has enabled researchers to make ratiometric measurements of intracellular Ca²⁺ using confocal laser-scanning microscopy or flow cytometry. For ratiometric measurements, fluo-3 can also be co-loaded into cells with a Ca²⁺-insensitive dye. For instance, carboxy SNARF-1 AM, acetate—a dye that can be excited at the same wavelengths as fluo-3 but detected at much longer wavelengths—can serve as the Ca²⁺-insensitive dye, provided that the pH within the cells remains constant during the experiment. SNARF-4F carboxylic acid, which can be loaded into cells as its AM ester, has a lower pKa of ˜6.4, likely making it the preferred probe for this application. Co-loading of fluo-3 and carboxy SNARF-1 also permits the simultaneous imaging of Ca²⁺ transients and intracellular pH in experiments in which the concentrations of both ions are changing.

Fluo-4

Fluo-4 is an analog of fluo-3 with the two chlorine substituents replaced by fluorine atoms exhibits a Kd for Ca²⁺ of 345 nM. The fluorescence quantum yields of Ca²⁺-bound fluo-3 and fluo-4 are essentially identical. The absorption maximum of fluo-4 is blue-shifted about 12 nm compared to fluo-3, resulting in increased fluorescence excitation at 488 nm and consequently higher signal levels for confocal laser-scanning microscopy flow cytometry and microplate screening applications.

Intracellular Ca²⁺ measurements using fluo-3 have become preferred for certain types of high-throughput pharmacological screening. Applications of this technology include screening for compounds that affect G-protein-coupled receptors, and identifying receptors for ligands known to be pharmacologically active. The stronger fluorescence signals provided by fluo-4 are particularly advantageous in cell types such as human embryonic kidney (HEK 293) cells, which are seeded at low densities for pharmacological screening assays.

Rhod-2 and X-Rhod-1

The long-wavelength Ca²⁺ indicators rhod-2 and X-rhod-1 are valuable for experiments in cells and tissues that have high levels of autofluorescence, and also for detecting Ca²⁺ release generated by photoreceptors and photoactivatable chelators. Rhod-2 was originally reported to exhibit only a three- to four-fold enhancement of fluorescence upon binding Ca²⁺. X-rhod-1 is a Ca²⁺ indicator with excitation/emission maxima of ˜580/602 nm and a Kd for Ca²⁺ of 700 nM. It has spectral characteristics that are similar to Calcium Crimson indicator, but the fluorescence response of X-rhod-1 is much more sensitive to Ca²⁺ binding. The long-wavelength emission characteristics of X-rhod-1 allow simultaneous detection Ca²⁺ transients and green-fluorescent protein (GFP) with minimal crosstalk.

Low-Affinity Calcium Indicators Based on Fluo-3 and Rhod-2

With Ca²⁺ dissociation constants well above 1 μM, low-affinity Ca²⁺ indicators can be used to detect intracellular Ca²⁺ levels in the micromolar range—levels that would saturate the response of fluo-3 and rhod-2. Such elevated Ca²⁺ levels are generated by mobilization of intracellular Ca²⁺ stores and by excitatory stimulation of smooth muscle and neurons. Moreover, low-affinity indicators have faster ion dissociation rates, making them more suitable for tracking the kinetics of rapid Ca²⁺ fluxes than indicators with Kd values of Ca²⁺<1 μM.

Fluo-5F, Fluo-4FF, Fluo-5N and Mag-Fluo-4

Fluo-5F, fluo-4FF, fluo-5N and mag-fluo-4 (Fluo Calcium Indicators) are analogs of fluo-4 with much lower Ca²⁺-binding affinity, making them suitable for detecting intracellular Ca²⁺ levels in the 1 μM to 1 mM range. Fluo-5F, fluo-4FF, fluo-5N and mag-fluo-4 have Kd values for Ca²⁺ of ˜2.3 μM, ˜9.7 μM, ˜90 μM and ˜22 μM, respectively, as compared to fluo-4, which has a Kd for Ca²⁺ of ˜345 nM. These low Ca²⁺-binding affinities are ideal for detecting high concentrations of Ca²⁺ in the endoplasmic reticulum and neurons, as well as for tracking Ca²⁺ flux kinetics.

Like fluo-4, these indicators are essentially nonfluorescent in the absence of divalent cations and exhibit strong fluorescence enhancement with no spectral shift upon binding Ca²⁺. Because mag-fluo-4 is less Ca²⁺/Mg²⁺ selective than fluo-5N, it is also useful as an indicator for intracellular Mg²⁺ levels.

Rhod-5N

Rhod-5N has a lower binding affinity for Ca²⁺ than any other BAPTA-based indicator (Kd=˜320 μM) and is suitable for Ca²⁺ measurements from 10 μM to 1 mM. Like the parent rhod-2 indicator, rhod-5N is essentially nonfluorescent in the absence of divalent cations and exhibits strong fluorescence enhancement with no spectral shift upon binding Ca²⁺. Furthermore, rhod-5N has very little detectable response to Mg²⁺ concentrations up to at least 100 mM. Rhod-5N is available as a cell-impermeant potassium salt or as a cell-permeant AM ester.

Rhod-FF, X-Rhod-5F and X-Rhod-FF

The fluorinated analogs of rhod-2—rhod-FF X-rhod-5F and X-rhod-FF—have intermediate Ca²⁺ sensitivity relative to rhod-2 and mag-rhod-2. Their Ca²⁺ dissociation constants (Kd) are 19 μM, 1.6 μM and 17 μM, respectively, compared to ˜0.57 μM for rhod-2 and ˜70 μM for mag-rhod-2.

iv. Magnesium Indicators

Intracellular Mg²⁺ is important for mediating enzymatic reactions, DNA synthesis, hormonal secretion and muscular contraction. To facilitate the investigation of magnesium's role in these and other cellular functions, several different fluorescent indicators for measuring intracellular Mg²⁺ concentration have been developed, including furaptra, (mag-fura-2); mag-indo-1, and mag-fura-5. For applications such as confocal laser-scanning microscopy and flow cytometry, Magnesium Green and mag-fluo-4 are preferred indicators.

Mg²⁺ indicators are generally designed to maximally respond to the Mg²⁺ concentrations commonly found in cells, typically ranging from about 0.1 mM to 6 mM. Intracellular free Mg²⁺ levels have been reported to be ˜0.3 mM in synaptosomes, 0.37 mM in hepatocytes and 0.5-1.2 mM in cardiac cells, whereas the concentration of Mg²⁺ in normal serum is ˜0.44-1.5 mM. Mg²⁺ indicators also bind Ca²⁺. However, typical physiological Ca²⁺ concentrations (10 nM-1 μM) usually do not interfere with Mg²⁺ measurements because the affinity of these indicators for Ca²⁺ is low. Although Ca²⁺ binding by Mg²⁺ indicators can be a complicating factor in Mg²⁺ measurements, this property can also be exploited for measuring high Ca²⁺ concentrations (1-100 μM).

Magnesium Indicators Excited by UV Light (Mag-Fura-2, Mag-Fura-5 and Mag-Indo-1)

The dissociation constants for Mg²⁺ of mag-fura-5 and mag-indo-1 are 2.3 mM and 2.7 mM, respectively, slightly higher than that of mag-fura-2, which is 1.9 mM. Mag-fura-2 was first used to detect Mg²⁺ fluctuations in embryonic chicken heart cells and rat liver cells. The lower-affinity mag-fura-5 and mag-indo-1 indicators are sensitive to somewhat higher spikes in intracellular Mg²⁺. The affinities of mag-fura-2 and mag-indo-1 for Mg²⁺ are reported to be essentially invariant at pH values between 5.5 and 7.4 and at temperatures between 22° C. and 37° C.

As with their Ca²⁺-indicating analogs, mag-fura-2 undergoes an appreciable shift in excitation wavelength upon Mg²⁺ binding, and mag-indo-1 exhibits a shift in both its excitation and emission wavelengths.

Equipment, optical filters and calibration methods are very similar to those required for the Ca²⁺ indicators. The excitation-ratioable mag-fura-2 and mag-fura-5 indicators are most useful for fluorescence microscopy, whereas the emission-ratioable mag-indo-1 indicator is preferred for flow cytometry. Simultaneous flow cytometric measurements of Ca²⁺ and Mg²⁺ have been made using fluo-3 and mag-indo-1.

Magnesium Indicators Excited by Visible Light (Magnesium Green and Mag-Fluo-4)

Several visible light-excitable Mg²⁺ indicators, including Magnesium Green and mag-fluo-4 indicators are available. As with mag-fura-2, mag-fura-5 and mag-indo-1, these visible light-excitable Mg²⁺ indicators can also be used as low-affinity Ca²⁺ indicators and may be useful as indicators for Zn²⁺ and other metals.

Magnesium Green indicator exhibits a higher affinity for Mg²⁺ (Kd ˜1.0 mM) than does mag-fura-2 (Kd ˜1.9 mM) or mag-indo-1 (Kd ˜2.7 mM). This indicator also binds Ca²⁺ with moderate affinity (Kd for Ca²⁺ in the absence of Mg²⁺˜6 μM). The spectral properties of the Magnesium Green indicator are similar to those of the Calcium Green indicators. Upon binding Mg²⁺, Magnesium Green exhibits an increase in fluorescence emission intensity without a shift in wavelength.

The Magnesium Green indicator has been used to investigate the binding of free Mg²⁺ by the bacterial SecA protein and by protein tyrosine kinases. By exploiting the fact that ATP has greater Mg²⁺-binding affinity than ADP, researchers have used Magnesium Green to detect ATP hydrolysis in spontaneously contracting cardiomyocytes.

Mag-fluo-4 is an analog of fluo-4 with a Kd for Mg²⁺ of 4.7 mM and a Kd for Ca²⁺ of 22 μM, making it useful as an intracellular Mg²⁺ indicator as well as a low-affinity Ca²⁺ indicator. Mag-fluo-4 has a much more sensitive fluorescence response to Mg²⁺ binding than does our Magnesium Green indicator. Because physiological fluctuations of intracellular Mg²⁺ concentration are typically small, this increased sensitivity is a considerable advantage. Like fluo-4, mag-fluo-4 is essentially non-fluorescent in the absence of divalent cations and exhibits strong fluorescence enhancement with no spectral shift upon binding Mg²⁺. Mag-fluo-4 is available as a cell-impermeant potassium salt or as a cell-permeant AM ester.

Methods of Measurement and Screening

Suitable instrumentation for measuring intracellular ion changes based on the use of ion sensitive indicators via optical methods includes microscopes, multiwell plate readers and other instrumentation that is capable of rapid, sensitive ratiometric fluorescence detection. A preferred instrument of this type is described in U.S. Pat. No. 6,349,160, described above.

### EXAMPLES

General Methods

β-Lactamase Measurements.

Unless otherwise noted, β-lactamase reporter expression was determined by plating cells into 96 well plates at approx. 10⁵ cells/well, and loading with 1 μM CCF2-AM in Hanks' balanced salt solution (HBSS) containing phenol red at 22 ° C. for a period between 1 and 2 h. For visual determination, an epifluorescence microscope (Zeiss 25CFL) equipped with a 10× Fluor objective was used. The filter set contained a 405/20 exciter, a 420 dichroic mirror, and a 435 long-pass filter (Chroma Technologies, Brattleboro Vt.). Fluorescence was then quantified on a Cytofluor 4000 fluorescence plate reader using 395/12.5 nm excitation and emission was detected via 460/50 (blue) and 535/40 (green) band pass filters. Emission intensities from blank wells containing no cells were subtracted from wells containing cells. Calibration of β-lactamase enzyme levels were determined in cell lysates as described by Zlokarnik et al.(1998) Science 279 84-88. Replicate samples of cells were lysed by three cycles of freeze thawing in PBS. β-Lactamase activity in the lysate was measured from the rate of hydrolysis of 1 nmol of CCF2 in 100 μl in a Cytofluor fluorimeter.

Flow Cytometry.

Flow cytometry and cell sorting were conducted using a Becton Dickinson FACS™ Vantage™ with a Coherent Enterprise II™ argon laser producing 60 mW of 351-364 nm multi-line UV excitation. The flow cytometer was equipped with pulse processing and the Macrosort™ flow cell. Cells were loaded with 1 μM CCF2/AM for 1-2 h prior to sorting, and fluorescence emission was detected via 460/50 nm (blue) and 535/40 nm (green) emission filters, separated by a 490 nm long-pass dichroic mirror. Using the Automatic Cell Deposition Unit™ (ACDU™) on the FACS™ Vantage™, single cells were sorted into 96-well microtiter plates based on relative blue and green fluorescence from the β-lactamase substrate CCF2.

### Example 1

### Construction of MC4R Specific DNA Construct

The pKI-CMV-MC4R construct was derived from a pBluescript (Stratagene, La Jolla, Calif.) backbone containing the PGKhygro cassette cloned into the Eco RI and Hind III sites and designated pPGK-hygro. The CMV-promoter was amplified from pcDNA3.1/neo (Invitrogen, San Diego, Calif.) with primers CMV-1F (5′-CTAGACGTTGACATTGATTATTGAC-3′) (SEQ ID NO:29) and CMV-2R (5′-TCTAGAGCCAGTAAGCAGTGGGTTC-3′) (SEQ ID NO:30) and standard amplification procedures.

The PCR product was cloned into vector pCR2.1-TOPO (Invitrogen, San Diego, Calif.) and the correct sequence verified. The CMV promoter was then subcloned into pPGK-hygro via Xba I operably linking the CMV-promoter to direct the expression of a gene fragment into the opposite direction of the PGK promoter. To produce the homology regions of the targeting construct, genomic DNA was isolated from cell line P3D8 containing a 4XCRE-β-lactamase reporter gene with the QIAmp DNA Mini Kit (QIAGEN, Valencia, Calif.). P3D8 is based on HEK 293 (human embryonic kidney cell line 293, ATCC CRL-1573, ATCC Rockville, Md.).

The 3′ homology arm was generated with the primer pair MC4R-3F (5′-GCGGCCGCCCATTGCATTGGGATTGGTC-3′) (SEQ ID NO:31) and MC4-4R (5′-GCGGCCGCCCTCCAAGTCTTTATCTG-3′) (SEQ ID NO:32). Both primers contain an artificial Not I at the 5′ end. The PCR product has a length of approximately 450 bp and is homologous to the 5′ untranslated region of the MC4 receptor exon. The fragment was amplified from 250 ng genomic DNA with the High Fidelity Platinum PCR Kit (Invitrogen, San Diego, Calif.) under the following PCR conditions: denaturation, 94° C. for 30 sec, annealing 58° C. for 30 sec and extension 72° C. for 1 min for 35 cycles. The PCR product was purified with the QIAquick PCR Purification Kit (Qiagen, Valencia Calif.) cloned into pCR2.1-Topo (Invitrogen, San Diego, Calif.) and the sequence verified.

For the 5′ arm of homology the following primers pair were employed MC4-1F, containing a Kpn I site (5′-GTACCGGCTCGTAGAGAAATATGAACC-3′) (SEQ ID NO:33) and MC4-2R, harboring an Xho I site (5′-CTCGAGAGAGACTGAATTTCCCTTTT-3′) (SEQ ID NO:34). PCR amplification with 250 ng genomic DNA was performed with the Advantage-GC Genomic PCR Kit (Clontech, Palo Alto, Calif.) and 1.5 M GC-Melt using the following conditions, denaturation, 94° C. for 45 sec, annealing 42° C. for 45 sec and extension 68° C. for 5 min for 5 cycles followed by 35 cycles of denaturation, 94° C. for 45 sec, annealing at 62° C. for 45 sec and extension at 68° C. for 5 min. The 4.5 kb PCR-product corresponding to the promoter region of the MC4R gene was purified by agarose gel electrophoresis with the QIAquick Gel Extraction Kit (QIAGEN, Valencia, Calif.), cloned into pCR2.1-TOPO (Invitrogen, San Diego, Calif.) and the sequence verified.

To obtain the targeting vector the 3′ homology arm was inserted into pKI-CMV via Not I, and the 5′ arm via Kpn I/Xho I. The resulting plasmid pKI-CMV-MC4R was linearized by Kpn I digestion.

### Example 2

### Construction of Growth Hormone Receptor-Specific DNA Construct

In another approach, one arm of homology of human growth-hormone releasing hormone receptor (GHRHR) was amplified and cloned into pcDNA3.1/hygro (Invitrogen, San Diego, Calif.) to obtain vector pKI-GHRHR-A (SEQ ID NO:46). Primers GHRHR-3F (5′-GCGGCCGCGAAGGAAGATAGCCAAGGCTTA-3′) (SEQ ID NO:35) and GHRHR-4R (5′-GCGGCCGCTTAAAGATGCCACACTGCTGGTCT-3′) (SEQ ID NO:36) were designed containing Not I sites at the 5′end. The amplification product of these primers comprised Exon 1 containing the ATG and parts of Intron 1 with a length of approx. 4.2 kb. Amplification was performed with 250 ng genomic DNA using the Advantage-GC Genomic PCR Kit (Clontech, Palo Alto, Calif.) and 1.5 M GC-Melt under the following amplification conditions: denaturation, 94° C. for 45 sec; annealing, 42° C. for 45 sec; and extension, 72° C. for 7 min for 5 cycles followed by 35 cycles of denaturation, 94° C. for 45 sec; annealing, 62° C. for 45 sec; and extension, 72° C. for 7 min. The PCR-product was gel purified with the QIAquick Gel Extraction Kit (QIAGEN, Valencia, Calif.), cloned into pCR2.1-TOPO (Invitrogen, San Diego, Calif.), the sequence verified, and the fragment subcloned into pcDNA3.1-hygro (Invitrogen, San Diego, Calif.) via Not I sites. The correct orientation of the fragment was confirmed by restriction digest and sequencing analysis. The construct was then linearized by by digestion with Xho I.

Alternatively, the arm of homology was cloned into the basal vector pKI-CMV and used as such or a 5′ region of homology added using, for example; the primer pair GHRHR-1F (5′-GTCGACACCTGTCGGCTACTGGGATA-3′) (SEQ ID NO:37) and GHRHR-2R (5′-GATATCGTGGGACTCTGTTTCCAGCA-3′) (SEQ ID NO:38) containing Sal I and EcoR V sites respectively. The resulting PCR product had a length of approx. 1 kb and is homologous to the promoter area of the GHRHR gene. The fragment is cloned into pCR2.1-TOPO (Invitrogen, San Diego Calif.) and the sequence verified. Following verification, the fragment was introduced into pKI-CMV containing the 3′ homology fragment via Sal I/EcoR V digest. This vector was linearized by digestion with Sal I

### Example 3

### Construction of Inducible DNA Constructs

The constructs in Examples 1 and 2 can be modified to allow regulated expression by replacing the CMV promoter with an inducible promoter. This is useful, for example, if constitutive expression of a target gene is toxic or inhibits growth of the cells. This is also useful for those embodiments where “activation” occurs simply by expression of the polypeptide following recombination. And such a construct is useful in the uninduced state as a negative screening control in order to determine what affect on the signal transduction detection system is target-specific.

### Example 4

### Construction of Nuclear Receptor Gal4-DBD DNA Constructs

Plasmid constructs were made for in situ creation of fusion proteins in which the Gal4-DBD was fused to the ligand-binding domain of a target nuclear receptor. The Gal4-DBD cDNA was PCR amplified using primers

| | |
|---|---|
| 5′CGGGTCCCCGGCGATACAGTCAACTGTCT3′ | (SEQ ID NO:43) |
| and | |
| 5′TAAAGCTTGCCACCATGAAGCTACTGTCTT3′. | (SEQ ID NO:44) |

The PCR fragment generated with these primers added a Kpn I site on the 5′ end and a Hind III site on the 3′ end of the Gal4-DBD. The resulting PCR fragment was digested with Kpn I and Hind III and cloned into pcDNA3. 1.

The resulting plasmid was named pCDGal4-DBD (SEQ ID NO:40). It contains multiple cloning sites immediately 3′ of the Gal4-DBD sequence. This plasmid was further modified by insertion of genomic sequences from one of four nuclear receptors such that when the plasmid inserts into the genome by homologous recombination, a polypeptide comprising the Gal4-DBD fused to the LBD of the targeted nuclear receptor iwas translated. In an alternative approach, the Gal4-DBD was amplified with and cloned into pKI-CMV-EYFP (SEQ ID NO:39) via Sma I/Xba I resulting in vector pKI-CMV-Gal4-EYFP (SEQ ID NO:49).

Construction of pKI-Gal4-DBD-PPARα (SEQ ID NO:41)

To obtain targeting vector pKI-Gal4-DBD-PPARα (SEQ ID NO:41) a 2.8 kb genomic fragment is amplified using the primer pair: (5′GCTCTAGAGGACGAATGCCAAGATCTGA3′) (“PPARaFor”; SEQ ID NO:62) and (5′CAAGCGGCCGCCAGTGTGATGGATATCTG 3′) (“PPARaRev”; SEQ ID NO:63) as described above. This PCR fragment comprises part of exon 6 of PPARα and the corresponding intron sequences. The PCR fragment is cloned into pCR2.1 Topo, the sequence confirmed and finally placed into pKI-CMV-Gal4-EYFP (SEQ ID NO:49) via the Xba I/Not I restriction sites to produce the final vector pKI-Gal4-DBD-PPARα (SEQ ID NO:41). This vector is linearized with Sfi I prior to electroporation.

Construction of pKI-Gal4-DBD-PPARγ (SEQ ID NO:51)

To obtain targeting vector pKI-Gal4-DBD-PPARγ a 2.9 kb genomic fragment of PPAR gamma is amplified using primer pair (5′ATATGGTACCGCAGTGGGGATGTCTCATAATGG′) (“PPARgFor”; SEQ ID NO:56) and (5′ AATTGGATCCTCAATCAGTCCATCACCTGG 3′) (“PPARgRev”; SEQ ID NO:57) as described above. This PCR fragment comprises part of exon 6 and the corresponding intron sequences of PPAR gamma. The PCR fragment is cloned into pCR2.1 Topo, the sequence confirmed and placed into pCDGal4-DBD (SEQ ID NO:40) via the Kpn I/Bam HI restriction sites to make pCDGal4-DBD-PPARγ (SEQ ID NO:42). The Gal4-DBD-PPARγ targeting sequence is then digested with Nhe I/Not I and finally transferred into pKI-CMV-EYFP (SEQ ID NO:39) cut with Xba I/Not I to produce pKI-Gal4-DBD-PPARg. This vector is linearized with Sfi I prior to electroporation.

Construction of pKI-Gal4-DBD-LXR (SEQ ID NO:50)

To obtain targeting vector pKI-Gal4-LXR (SEQ ID NO:50) a 3.8 kb genomic fragment of LXR is be amplified using primer pair (5′ CGTCTAGAGAGTGTGTCCTGTCAGAAGAAC 3′) (“LXR-1Fg”; SEQ ID NO:58) and (5′ ATGCGGCCGCACTCCTGACCTCAGGTGATCC 3′) (“LXR-2Rg”; SEQ ID NO:59) as described above. This PCR fragment comprises part of exon 4, exon 5, exon 6 and the corresponding intron sequences of the LXR gene. The PCR fragment was cloned into pCR2.1 Topo, the sequence confirmed and subcloned into pKI-CMV-Gal4-EYFP (SEQ ID NO:49) via Xba I/Not I to produce the vector pKI-Gal4-DBD-LXR. This vector is linearized with Sfi I for electroporation into recipient cells.

Construction of pKI-Gal4-DBD-FXR (SEQ ID NO:52)

To produce vector pKIGal4-DBD-FXR (SEQ ID NO:52) a 3.1 kb genomic DNA fragment of the FXR gene comprising the the start of the LBD, Exon 7, Exon 8 and the respective introns is used. To amplify this genomic sequence primer pair (5′CGTCTAGAGAAGACAGTGAAGGTCGTGAC 3′) (“FXR-5Fg”; SEQ ID NO:60) and (5′ GCGCGGCCGCGTCTAACCTAGGAGCCCAC 3′) (“FXR-6Rg”; SEQ ID NO:61) was used. After amplification the PCR-product was cloned into pCR2. 1 Topo as described above and the sequence confirmed. The fragment was then subcloned using Xba I and Not I and inserted into pKI-CMV-Gal4-EYFP (SEQ ID NO:49). The vector was linearized for electroporation using Sfi I.

### Example 5

### Generation and Validation of MC4R Assay

Plasmid pKI-MC4R (SEQ ID NO:45) was linearized with Kpn I and introduced into cell line P3D8 containing a cyclic AMP respose element (CRE) configured to direct the β-lactamase reporter by electroporation. Cells were grown in DMEM with 4.5 mg/l glucose, L-glutamine and 25 mM HEPES, pH 7.6 (Invitrogen, San Diego, Calif.) supplemented with the following reagents: 10% fetal calf serum; 1× penicillin/streptomycin; 1× MEM-non-essential amino acids; 1× MEM-sodium pyruvate (DMEM complete) and 300 μg/ml Geneticin (all from Invitrogen, San Diego, Calif.). Electroporations were performed using a Gene Pulser II combined with a Capacitance Extender Plus (BioRad, Hercules, Calif.). Specifically, cells were removed from flasks, dissociated, centrifuged and resuspended into CytoMix (10 mM KCl; 0.15 mM CaCl₂; 10 mM K₂HPO/H₂PO₄, pH 7.6; 25 mM HEPES, pH 7.6; 2 mM EGTA; 5 mM MgCl₂) at a concentration of 1×10 cells/ml. Fifteen μg of linearized vector was added and 10 separate electroporations performed at 0.325 kV and 1.07 F. Cells were then replated in 150mm plates in DMEM complete media with 30 ug/ml hygromycin at a density suitable for selection. Cells harboring the targeting construct were selected for resistance to 30 μg/ml hygromycin (Roche, Indianapolis, Ind.). Based on serial dilutions of the initial electroporation mix, it was estimated that approximately 500,000 individual cell clones were obtained from the 10 electroporations.

Cells were prepared for FACS to identify cells where the β-lactamase reporter gene expression was inducible on the addition of the MC4 agonist. Cells were plated on Matrigel (Becton Dickenson, San Jose, Calif.) coated flasks the day prior to the FACS procedure at 50-60% confluence.

The following morning the media was changed to DMEM medium containing 1% FCS and the cells were incubated for 4 h with NDP-α-MSH (Phoenix Pharmaceuticals, Belmont, Calif.) at a concentration of 100 nM. Cells were removed from the flask, counted, centrifuged, resuspended into sorting buffer (PBS with 0.1 gram/liter Ca²⁺, 1.0% glucose, 1 mM EDTA, and 25 mM HEPES, pH 7.6) with 2 μM CCF2AM (Vertex Pharmaceuticals (San Diego), San Diego, Calif.) (made by adding 2 μls of 2mM CCF2-AM in dry DMSO into 16 μl 100 mg/mL Pluronic-F127 in DMSO per ml of sorting buffer) at a concentration of 0.8×10⁶ cells/ml and passed through a 40 μm cell strainer. The flow through was collected, placed into a T-75 flask and incubated on a shaker platform at 60 rpm for 1 h at room temperature in the dark. Cells were collected by centrifugation and resuspended in sorting buffer (PBS with 0.1 gram/liter Ca²⁺, 1.0% glucose, 1 mM EDTA and 25 mM HEPES, pH 7.6) at a concentration between 2.0-5.0×10⁶ cells/ml.

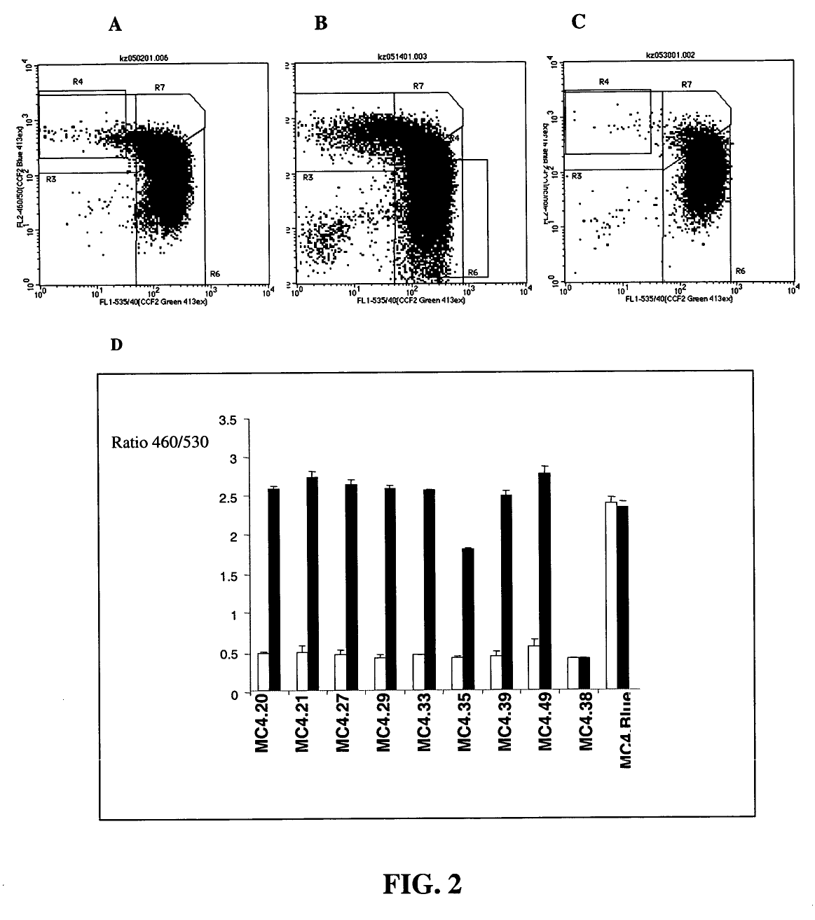

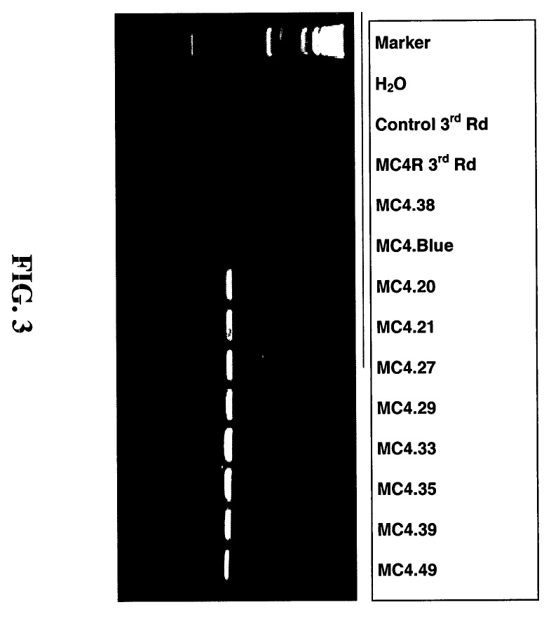

Cells were subjected to three consecutive rounds of FACS to enrich for cells harboring an activated MC4R gene with a Vantage SE (Becton Dickinson, San Jose, Calif.) (FIG. 2). Cells expressing β-lactamase converted the substrate, CCF2-AM, to a blue-fluorescent compound while cell without β-lactamase activity remain green-fluorescent indicating the presence of uncleaved substrate.

Cells were then resorted as described above, but no stimulus was added. During this round 1.3×10⁶ cells or 36.8% (green fluorescent) out of 6.3×10⁶ cells were sorted and collected. After expansion these cells were subjected to a third round of FACS sorting, executed as described for the first round except that single cells were sorted individually into each well of 20 separate 96 well plate that were previously coated with Matrigel (Becton Dickenson, San Jose, Calif.) and filled with 100 μl of media containing DMEM with 20% fetal bovine serum. For this sort 0.3×10⁶ cells (0.32% of total blue fluorescent cells) were selected from a total of 5.2×10⁶ cells. Selection media was added to each well two days after sorting.

After selection 350 distinct clones were dissociated from their 96 well plate and transferred onto new 96-well plates. Duplicate plates were generated by dissociating cells in the first set of plates and plating the cells into two duplicate sets of plates. The first plate represented a master plate while the second plate was used to identify cell clones expression MC4 receptor. To this end, after reaching near confluence, media was removed from the individual wells, cells were washed once with DMEM containing 1% fetal bovine serum and two duplicate wells were plated. One plate was stimulated, the other left untreated. Specifically, 100 μl of DMEM with 1% FBS (with or without NDP-α-MSH (100 nM)) was added to each of the duplicate wells. Cells were incubated for 4 h at 37° C. prior to assaying for β-lactamase activity.

To detect β-lactamase activity a 6×CCF2-AM loading solution was made by adding 6 μl of 2 mM CCF2-AM in dry DMSO into 60 μl 100mg/mL Pluronic-F127 in DMSO containing 0.1% acetic acid. Sixty-six μl of this resulting solution was then added to 1 ml of 24% (w/w) PEG400 with 12% ESS (40 mM Tartrazine and 40 mM NT-Red40 from Noveon-Hilton Davis, Cincinnati, Ohio) in water. This was then added to 100 μl of cells that had been preincubated in either the presence or absence of stimulus. Cells were then incubated for 1 h at room temperature. β-lactamase activity was then quantified using ratiometric readout (460/40 nm excitation filter; 530/30 nm emission filter) on a Cytoflor 4000 plate reader (Perseptive Biosystems, Framingham, Mass.). The activity from the unstimulated cells was compared to that of the stimulated cells (FIG. 2, panel D).

Fifty-one out of the 350 clones tested positive for β-lactamase activity after stimulation of MC4R ligand. The six positive clonal cell lines with the largest dynamic range were selected for molecular characterization. Genomic DNA was isolated each cell line using the QIAmp DNA Mini Kit (QIAGEN, Valencia, Calif.). 250 ng of genomic DNA, was taken and PCR reactions were set up with the following primers CMV-10R (5′-GAGAACCCACTGCTTACTGGCT-3′) (SEQ ID NO:47) and MC4-8Rg (5′-GCATTGCTGTGCAGTCTGTAA-3′) (SEQ ID NO:48). Primer CMV-10R binds to CMV-promoter while primer MC4-8Rg binds outside the targeting vector in the coding region of the exon 1 for MC4. The expected 960 bp PCR product was generated after amplification for 35 cycles with a protocol that included cycles of 94° C. for 45 sec to denature fragments, 57° C. for 45 sec for annealing and 72° C. for 1 min for extension from each cell line (FIG. 3). This PCR product was purified with the QlAquick PCR Purification Kit (QIAGEN, Valencia, Calif.) and cloned into pCR2.1-TOPO (Invitrogen, San Diego, Calif.). The size and sequence of the cloned PCR products confirmed the targeting vector had integrated into the genome by homologous recombination.

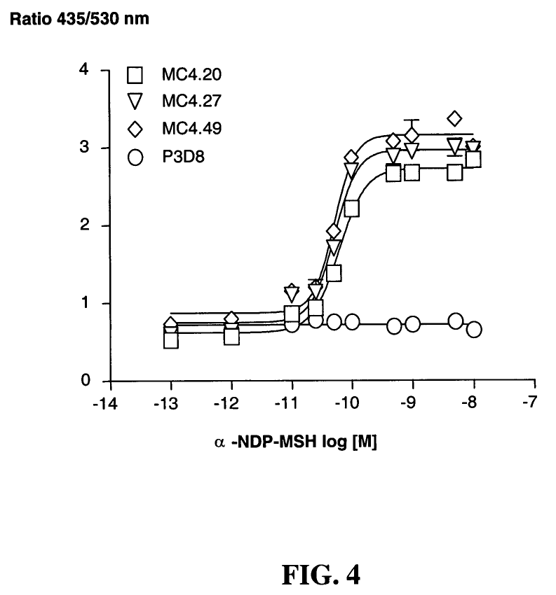

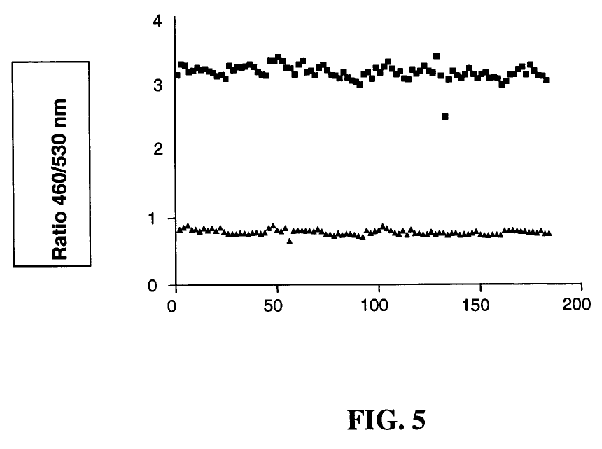

Three out of the eight characterized MC4 cell lines were chosen for further pharmacological characterization that included a dose response curve with NDP-α-MSH. All three cell lines displayed similar responses to the stimulus (FIG. 4). The calculated values were consistent with published data. Pretreating the cells with the MC4 antagonist HS024 showed that the response to either the natural ligand α-MSH or ligand NDP-α-MSH could be inhibited in a concentration-dependant manner. Based on these experiments clone MC4.49 was chosen for further assay development. To determine the assay validation ratio (AVR) a checkerboard assay was performed. To demonstrate compatibility of the assay for a high density-screening format including robotic handling the cell clone MC4.49 was analyzed in a dose response in miniaturized UHTSS format. Analysis of the AVR showed a robust performance of this assay (FIG. 5).

### Example 6

### Generation and Validation of GHRHR Assay

The pKI-GHRHR-A (SEQ ID NO:46) vector from Example 2 is linearized with Xho I and transfected into P3D8 cells by electroporation. Cells are grown in DMEM with 4.5 mg/l glucose, L-glutamine and 25 mM HEPES, pH 7.6 supplemented with 10% fetal bovine serum; 1× penicillin/streptomycin; 1× MEM-non-essential amino acids; 1× MEM-sodium pyruvate and 300 μg/ml Geneticin. Electroporations are performed using a Gene Pulser II combined with a Capacitance Extender Plus (BioRad, Hercules Calif.).

Specifically, cells are removed from the flasks by dissociation, centrifuged and resuspended into CytoMix (10 mM KCl, 0.15 mM CaCl₂, 10 mM K₂HPO₄/KH₂PO₄, 25 mM HEPES, 2 mM EGTA, 5 mM MgCl₂, pH 7.6) at a concentration of 1×10⁷ cells/ml and electroporated at 0.325 kV and 1.07 μF with 15 μg of linearized pKI-GBRHR-A (SEQ ID NO:46) plasmid DNA. A total of 10 electroporations are performed. Cells harboring the targeting construct were selected by adding 30 μg/ml hygromycin (Roche, Indianapolis, Ind.) and 300 μg/ml Geneticin to the media for two to three weeks.

Cells are prepared for sorting based on β-lactamase reporter gene read-out following stimulation with GHRH (Phoenix Pharmaceuticals, Belmont, Calif.) at a concentration of 100 nM. Specifically, cells were plated on Matrigel (Becton Dickenson, San Jose, Calif.)-coated flasks the day prior to the FACS sorting experiment at 50-60% confluence. The following morning the cells were washed with DMEM containing 1% fetal bovine serum. 100 nM GHRH was added in DMEM medium containing 1% fetal bovine serum. Cells were stimulated for 4 h, removed from the flask, and resuspended at a concentration of 0.8×10⁶ cells/ml into-Phosphate Buffered Saline (Invitrogen San Diego, Calif.)+1.0% glucose+1 mM EDTA and 25 mM HEPES, pH 7.6) containing 2 μM CCF2AM.

Cells were then passed through a 40 μm cell strainer, collected by centrifugation and resuspended in sorting buffer at a concentration between 2.0-5.0×10⁶ cells/ml. To enrich for cells harboring an activated GHRHR gene, four consecutive rounds of FACS sorting were performed using a FACS Vantage SE (Becton Dickinson, San Jose).

For FACS sorting, dye-loaded cells are excited with an argon laser and the emission detected via 460/50 nm (blue) and 535/40 nm (green) emission filters. Cells were sorted based on relative blue or green fluorescence from the β-lactamase substrate. During the first round, blue fluorescent cells following GHRH stimulation were collected from a total of 2×10⁸ cells. The collected cells were expanded prior to a second sort round of FACS where cells were not stimulated prior to sorting. During this round green fluorescent cells are collected from the total cells. After expansion the cells were subjected to a third round of FACS, executed as described for the first round. In parallel, single cells are sorted into 40 Matrigel (Becton Dickenson, San Jose, Calif.) coated 96-well plates containing DMEM plus 20% fetal bovine serum as described above. Two d after sorting, selection media was added to each well.

After selection, 450 distinct clones were were frown until reaching near confluence. The media was then removed from the individual wells, and the cells were washed once with DMEM containing 1% fetal bovine serum. The cells were then plated in two duplicate wells each on a different 96-well plate. One plate was stimulated by the addition of 100 μl of DMEM with 1% FBS containing either 10 or 100 nM GHRH (Phoenix Pharmaceuticals, Belmont, Calif.). The other plate was left untreated by adding just the media without any GHRH. Cells were then incubated for 4 h at 37°.

To detect β-lactamase activity, 66 μl of 6×CCF2-AM loading solution was added to 1 ml of 24% (w/w) PEG400 with 12% ESS (Vertex Pharmaceuticals (San Diego), San Diego Calif.) in water and the resulting solution added to 100 μl of cells that had been preincubated in either the presence or absence of stimulus. Cells were then incubated for 1 h at room temperature. β-lactamase activity was then quantified using ratiometric readout (460/40 nm excitation filter; 530/30 nm emission filter) on a Cytoflor 4000 plate reader (Perseptive Biosystems, Framingham, Mass.). The activity from the unstimulated cells was compared to that of the stimulated cells.

### Example 7

### Generation of Nuclear Receptor Gal4-DBD Assays

A HEK293 cell line was generated in which the UAS response element was linked to the β-lactamase reporter gene. Specifically, the 7×UAS response element (SEQ ID NO:53) was cloned in front of the β-lactamase reporter gene to create 7×UAS Bla-M (SEQ ID NO:64). Ten μg of the resulting UAS-BLA DNA was transfected into HEK293 cells and stable cell lines selected with DMEM containing 10% fetal bovine serum and 100 μg/ml Zeocin (Invitrogen, Carlsbad, Calif.). Single cells were isolated by FACS and expanded for further analysis. 60 single cell lines were tested by transient transfection with 10 μg of a VP16-Gal4-expressing plasmid vector (Clontech, Palo Alto, Calif.) followed by monitoring for β-lactamase expression 24 h after transfection. To monitor β-lactamase expression cells were loaded with 2 μM CCF2-AM and compared with untransfected control for each of the 60 cell lines tested. A single HEKUASBLA clone was selected with a 460 nm/530 nm ratio change from untransfected to transfected of about 5-fold.

Cells from this clonal cell line (HEKUASBLA) were transfected with the nuclear receptor-specific DNA constructs described in Example 4. Specifically, four plasmids (pKI-Gal4-DBD PPARα (SEQ ID NO:41), pKI-Gal4-DBD PPARγ (SEQ ID NO:42), pKI-Gal4-DBD LXR (SEQ ID NO:50), and pKI-Gal4-DBD FXR (SEQ ID NO:52)) were linearized and transfected into HEK cells to generate a populations of 100,000 independent stably transfected cells. In each case 1×10⁷ HEKUASBLA cells were electroporated with 15 μg of the individual DNA. Ten independent electroporations were done for each plasmid followed by selection in media containing DMEM, 10% fetal bovine serum and 100 μg/ml hygromycin. After between 2 and 3 weeks of selection the number of stable clones was counted using colony counting from serial diluted electroporations, and a population of 100,000 stable clones was mixed for FACS sorting using a FACS Vantage SE (Becton Dickinson, San Jose, Calif.). Cells were prepared for FACS by an overnight stimulation with a ligand specific for the individual nuclear receptor followed by loading with CCF2-AM for 1 h and sorting for blue fluorescent cells. Sorting for responsive cells was performed as described in Example 6 with substitution of the appropriate enzyme.

### Example 8

### Validation of PPARα Nuclear Receptor High Throughput Screen

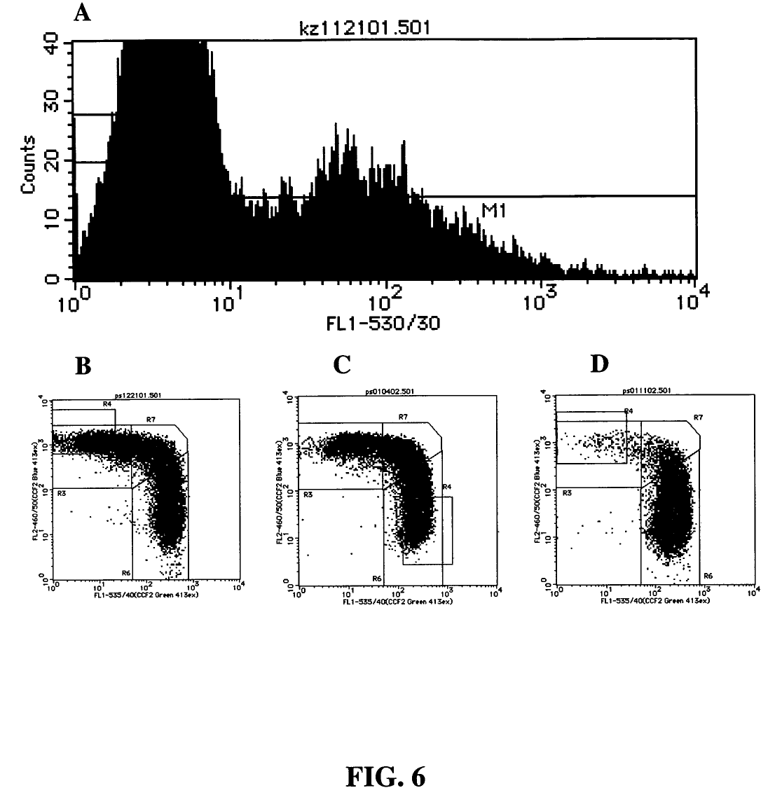

HEK UAS BLA-1 cells were electroporated with 15 μg linearized PCD-Gal4-DBD-PPARγ (SEQ ID NO:42) from the previous example. Ten independent electroporations were done for each plasmid followed by selection in media containing DMEM, 10% fetal bovine serum and 100 μg/ml hygromycin. After between 2 and 3 weeks of selection the number of stable cell lines was determined, using colony counting from serial diluted electroporations, and a population of approximately 100,000 stable clones was mixed for FACS sorting using a FACS Vantage SE (Becton Dickinson, San Jose, Calif.). Cells were prepared for FACS by stimulating 16 hours with 10 μM rosiglitazone followed by loading with CCF2-AM for 1 h and sorting for blue fluorescent cells. Sorting for responsive cells was performed as described in Examples 6 and 7 with substitution of the appropriate enzyme (FIG. 6).

In the first round of FACS sorting, EYFP negative cells were collected, (FIG. 6, panel A). These are cells that are negative for enhanced yellow fluorescent protein expression (Vertex Pharmaceuticals (San Diego), San Diego Calif.). This selection was intended to enrich for homologous versus random integration. From a total of 52×10⁶ cells, 13×10⁶ cells were obtained that did not express the fluorescent marker. These cells were allowed to recover and then two days prior to the FACS sorting based upon β-lactamase expression, the cells were serum-starved in phenol red free DMEM supplemented with 2% charcoal-stripped FCS. At least 16 h before FACS the agonist Rosiglitazone was added to the cells at a concentration of 10 μM. Sorting for β-lactamase expressing cells was performed as described in Examples 6 and 7 with substitution of the appropriate enzyme. During the first round 2.5×10⁵ or 8% of β-lactamase expressing cells were collected from a total of 32×10⁶ cells sorted, (FIG. 6, panel B). These cells were expanded and subjected to a second round of sorting as described in Example 6 (FIG. 6, panel C). At this time 30×10⁶ were sorted and 8×10⁶ or 26% were recovered. During the third and final round 15.8×10⁶ were sorted and 6.1×10⁴ or 0.4% were recovered (FIG. 6, panel D).

Single cells were sorted into 96 well plates and expanded. A total of 400 individual cells clones were picked and tested for responsiveness to the agonist rosiglitazone. To this end cell clones were transferred to a master plate and copied onto two assay plates. The next day or after cells had reached confluence the media was changed on the assay plates to DMEM-Assay. At least 16 h before the assay, cell clones on one copy of the assay plates were stimulated as described above. β-lactamase activity was detected in individual clones as described in Example 6. Out of the 400 cell lines tested 36 or 9% displayed β-lactamase activity with a dynamic range varying between 2- to 25-fold. Six cell lines displaying the most robust β-lactamase induction were selected for molecular characterization. Total RNA was extracted from each of the 6 cell lines and 2 non-inducible cell lines using the Qiagen RNeasy Total RNA Kit (Qiagen, Valencia, Calif.). cDNA was generated with the SuperScript First Strand Synthesis Kit (Invitrogen, Carlsbad, Calif.). PCR reactions were performed using primer CMV-10FR (5′ GAGAACCCACTGCTTACTGGCT 3′) (SEQ ID NO:54) binding to the CMV Promoter and primer PPARγ RT Rev3 (5° CAAGATCGCCCTCGCCTTTG 3′) (SEQ ID NO:55) corresponding to part of the LBD of PPARγ not included in the construct.

Analysis of the PCR reactions showed that all cell clones responding to Rosiglitazone yielded a PCR product while non-responding cell clones did not. Sequencing of the PCR products revealed the predicted sequence.

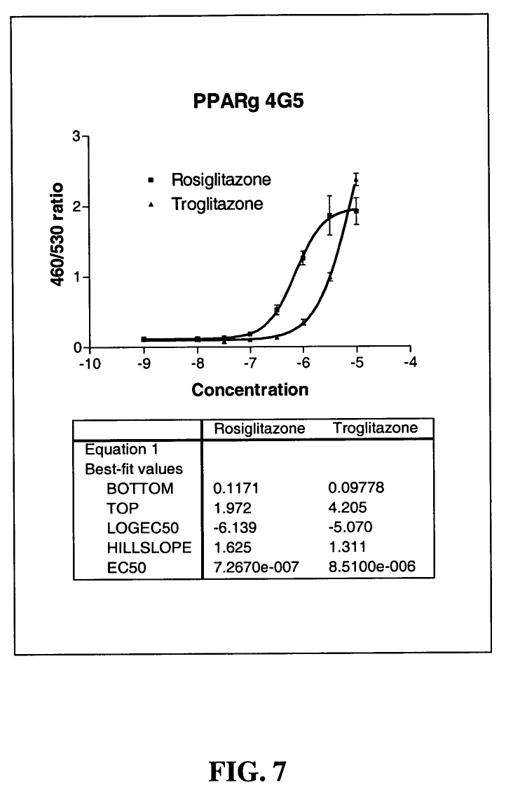

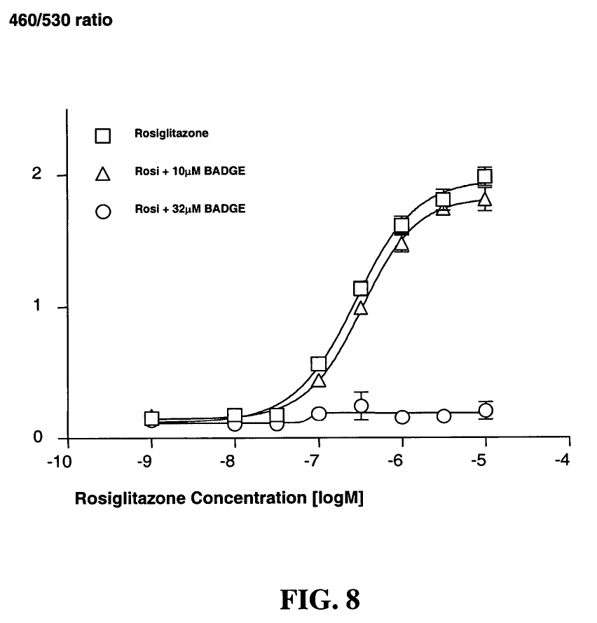

Based on these data, clone 4G5 was selected for assay development and validation. To determine the EC₅₀ a dose response curve was created with ligand rosiglitazone. The EC₅₀ was determined to be (430 nM) (FIG. 7). To further confirm the specificity of the signaling through the PPARγ-Gal4 fusion the Rosiglitazone-specific antagonist BADGE was employed. To this end cells were treated as described previously prior to addition of 10 μM, 32 μM or no BADGE. After a period of 1 h at 37° C. Rosiglitazone was added in increasing doses between 1 nM and 10 μM (FIG. 8). Inhibition of the cell response to the stimuli confirmed that β-lactamase expression is a result of PPARγ activation after ligand binding

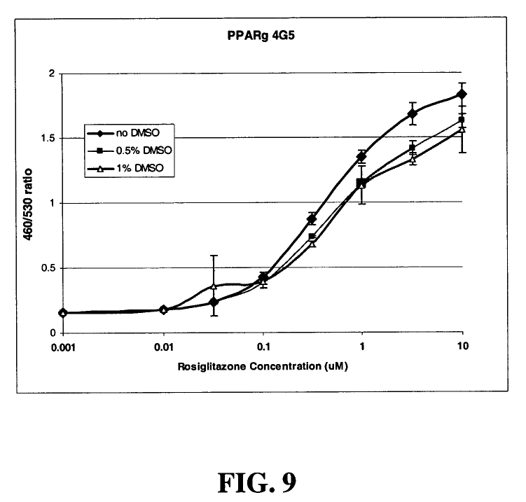

For miniaturized screening cells were set up the following manner. On day 1 cells were seeded onto a Matrigel coated flask. The following day the media was changed to DMEM-Assay media. On day 3 cells were harvested and plated into NanoWell™ plates at a density of 4000 cells/well. The NanoWell™ plates contained test compounds. After 16 h the cells were loaded with CCF4 as described above. β-lactamase ratiometric readout was determined using a topologically-corrected fluorescent plate reader (tc-PR)(FIG. 9).

This protocol was used to screen three independent compound libraries (as shown below). The assay validation ratio (AVR) is determined by the following formulae: 1.

$$\frac{3 ( {SD}_{sig} ) + 3 ( {SD}_{bas} )}{\vert Sig - Bas \vert} < 1.0 ;$$

wherein “Sig” is the mean 460 nm/530 nm ratio of the wells containing test compound; “Bas” is the mean 460 nm/530 nm ratio in the negative control wells (no test compound); “SD_{sig}” is the standard deviation for the test compound wells; and “SD_{bas}” is the standard deviation of the negative control wells. When this formula is satisfied, there will be a theoretically ≦1% false hit rate if the hit cutoff is set at the median value between the “sig” and “bas” readings.

| | Library Size | Dynamic Range | AVR | # of hits selected |
|---|---|---|---|---|
| Library 1 | 91,000 | 16.3 | 0.35 | 785 (0.86%) |
| Library 2 | 73,000 | 11.4 | 0.37 | 629 (0.86%) |
| Library 3 | 48,000 | 19.7 | 0.36 | 341 (0.71%) |

Out of the total of 1755 hits from the primary screen 1546 were selected for retesting. These 1546 hits were retested for dose response in the PPARγ assay and 1020 showed a dose response. Out of 1020 confirmed hits 810 were non-specific or fluorescent compounds. Therefore 210 compounds remained and are further evaluated in secondary assays.

### Example 9

### Construction of Additional Nuclear Receptor Gal4-DBD Constructs

This example teaches the construction of nuclear receptor GAL4-DBD DNA constructs for Nurr 1 (Nur-related receptor-1) Accession No. NM 006186, GR (Glucocorticoid receptor) Accession No. NM 000176, and MR (Mineralocorticoid receptor) Accession No. XM 055775. As taught in Example 4, plasmid constructs were made for in situ creation of fusion proteins in which the Gal4-DBD was fused to the ligand-binding domain of a target nuclear receptor.

Construction of pKI-Gal4-DBD-Nurr1

To obtain targeting, vector pKI-Gal4-DBD-Nurr1 a 2.4 kb genomic fragment for Nurr1 is amplified using primer pair (5′GGGGTACCAAAGAAGGTAGGCTGAGGGG 3′) (SEQ ID NO:65) (Nurr1For) and (5′ CGGGATCCGTACAAGACAGTTAGCTAGTTGGC 3′) (SEQ ID NO:66) (Nurr1Rev) as described above. This PCR fragment comprises part of exon 4, all of exons 5,6, and 7 and the corresponding intron sequences of Nurr1. The PCR fragment is cloned into pCR2.1 Topo, the sequence confirmed and placed into pCDGal4-DBD (SEQ ID NO:40) via the Kpn I/Bam HI restrictions sites to make pCDGal4-DBD-Nurr1. The Gal4-DBD-Nurr1 targeting sequence is then digested with Nhe I/Not I and finally transferred to pKI-CMV-EYFP (Seq ID NO:39) cut with Xba I Not I to produce pKI-Gal4-DBD-Nurr1. This vector is linearized with Sfi I prior to electroporation. As taught in Example 7, the linearized vector was then used to transfect cells from the clonal cell line (HEKUASBLA) by electroporation. Following the teachings of Example 7, a selection of suitable cells appropriate for high throughput screens were identified.

Construction of pKI-Gal4-DBD-GR

To obtain targeting vector pKI-Gal4-DBD-GR a 3.6 kb genomic fragment for the Glucocorticoid Receptor is amplified using primer pair (5′GGGGTACCATTCAGCAGGCCACTACAGGACTCTC 3′) (SEQ ID NO:67) (GR_For) and (5′ GATCGCGGCCGCTGTGCTCGACATTGGTGGCC3′) (SEQ ID NO: 68) (GR_Rev) as described above. This PCR fragment comprises part of exon 5, all of exon 6, and the corresponding intron sequences of GR. The PCR fragment is cloned into pCR2.1 Topo, the sequence confirmed and placed into pCDGal4-DBD (SEQ ID NO:40) via the Kpn I/Not I restrictions sites to make pCDGal4-DBD-GR. The Gal4-DBD-GR targeting sequence is then digested with Nhe I/Not I and finally transferred into pKI-CMV-EYFP (SEQ ID NO:39) cut with Xba I/Not I to produce pKI-Gal4-DBD-GR This vector is linearized with Sfi I prior to electroporation. As taught in Example 7, the linearized vector was then used to transfect cells from the clonal cell line (HEKUASBLA) by electroporation. Following the teachings of Example 7, a selection of suitable cells appropriate for high throughput screens were identified.

Construction of pKI-Gal4-DBD-MR

To obtain targeting vector pKI-Gal4-DBD-MR a 3.2 kb genomic fragment for the Mineralocorticoid Receptor is amplified using primer pair (5′ GGGGTACCTTTGTGGTGCTTAAAAAATGAGC 3′) (SEQ ID NO:69) (MR_For) and (5′ GATCGCGGCCGCCTCATGAACAATGAAATCTCC3′) (SEQ ID NO:70) (MR_Rev) as described above. This PCR fragment comprises part of exon 5, all of exon 6, and the corresponding intron sequences of MR. The PCR fragment is cloned into pCR2.1 Topo, the sequence confirmed and placed into pCDGal4-DBD (SEQ ID NO:40) via the Kpn INot I restrictions sites to make pCDGal4-DBD-MR. The Gal4-DBD-MR targeting sequence is then digested with Nhe I/Not I and finally transferred into pKI-CMV-EYFP (SEQ ID NO:39) to produce pKI-Gal4-DBD-MR. This vector is linearized with Sfi I prior to electroporation. As taught in Example 7, the linearized vector was then used to transfect cells from the clonal cell line (HEKUASBLA) by electroporation. Following the teachings of Example 7, a selection of suitable cells appropriate for high throughput screens were identified.

### Example 10

### Validation of Nurr 1 Nuclear Receptor High Throughput Screen

Strategy for Nurr1 Clone Isolation in the Absence of a Nurr 1 Ligand

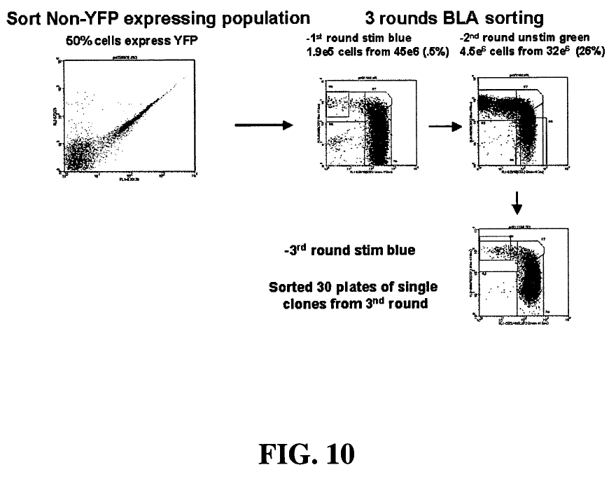

Nurr1 is documented to heterodimerize with the retinoid X receptor (RXR). In the absence of a known Nurr1 ligand, identification of Nurr1-responsive clones was done via response of Nurr1-RXR heterodimers to the RXR agonist, 9-cis-Retinoic Acid (9-cis RA). To this end, 9-cis RA was used to stimulate β-lactamase transcription from the GAL4-Nurr1/RXR heterodimer bound to the UAS promoter. Flow cytometry was employed to isolate clones expressing active GAL4-Nurr1/RXR heterodimers. The flow cytometry sorting strategy employed was as follows: first round, sort for YFP negative cells; second round, sort for 9-cis RA stimulated blue cells, third round, sort for unstimulated green cells and finally a fourth round of 9-cis RA stimulated blue cells (FIG. 10). This approach resulted in isolation of clones that are dependent on the functional activity of GAL4-Nurr1.

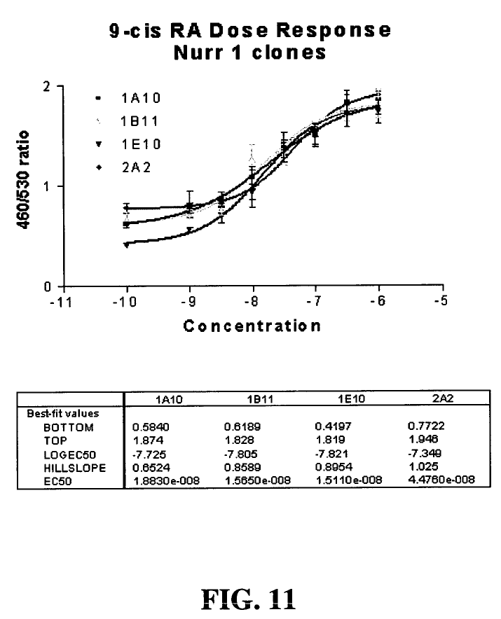

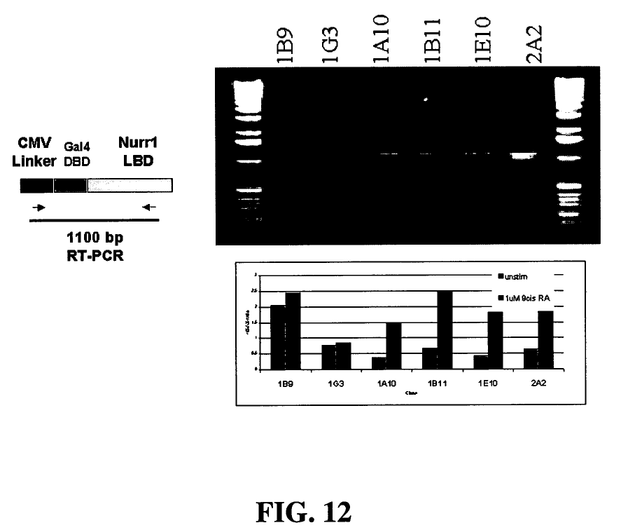

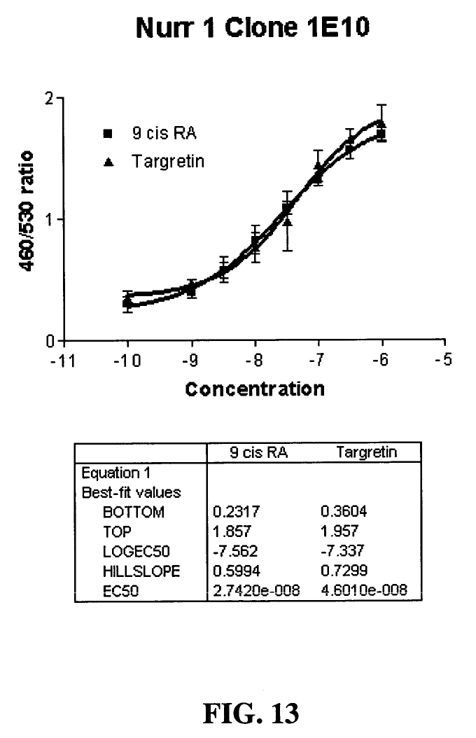

Individual HEK/UAS/GAL4-Nurr1 FACS sorted clones were expanded and quantitatively analyzed for β-lactamase expression. All 9-cis RA inducible clones were selected for further validation. Analysis of GAL4-Nurr1 integration into the genome was verified by RT-PCR with primers specific for targeted site of integration (FIG. 11 and FIG. 12). The best performing HEK/UAS/GAL4-Nurr1 clone, 1E10, was chosen for assay optimization and validation for ultra-high throughput screening (FIG. 13).

Cell Culture Conditions

Passage Conditions

Harvesting/Splitting Procedure for T-225 Flasks:

1. Coat new T-225 flask with 10 ml Matrigel; set aside at room temperature.

2. Aspirate media from flasks containing cells.

3. Wash cells with 10-20 ml PBS, aspirate.

4. Add 3 ml room temperature versene, rotate flask to cover cells with versene, let stand at room temperature for 2-5 min to dissociate cells.

5. Resuspend dissociated cells in 7 ml growth medium (10 mls total in flask). Triturate cells by pipetting briskly to remove clumps.

6. Split the cells at 1:10 or 1:15 if they are really dense. Use 30 ml of media per T-225. The cells will be confluent three to four days later.

7. Cells may be split at 1:2 to 1:10 depending on need. A flask at 80-90% confluency will yield approximately 20e6 cells per T-225 (˜1-7e6/ml).

Growth medium

DMEM (Gibco Cat #12430-054 which contains glucose, L-glut, 25 mM BEPES and pyridoxine HCl)

10% heat-inactivated and dialyzed FBS (55 mls/500 ml media)

1× Pen/Strep (5.5 ml Pen-Strep/500 ml media, Gibco cat #15140-122)

1 mM Sodium Pyruvate (5.5 ml 100 mM NaPyruvate/500 ml media, Gibco cat #11360-070)

1× Non-essential amino acids (5.5 ml NEAA/500 ml media, Gibco cat #11140-050)

Nanoplate Assay Validation

The assay was validated in the 3456 nanoplate format. The parameters tested include but are not limited to:
1) Cell density. The range of cell density tested was from 500 cells/well to 4000 cells/well. Optimal screening density was determined to be from 1500-2500 cells/well on the nanoplate assay. Use 2000 cells/well for nanoplate screening.
2) Culture time. Optimal culture time is between 16-24h. Allow 16-20h of incubation during screening.
3) Agonist concentration. 1 uM 9-cis RA stimulated cells act as positive controls during nanoplate screening.
4) CCF4 loading time. Use 1.5 h CCF4 loading time during screening.
5) DMSO sensitivity
6) Assessment of assay window in 3456 well-format with bioactive compound set Nanoplate experimental parameters used on the FRD:
   1) Assay media, DMEM, 0.1% BSA and P/S
   2) Cell stock, 1.7e6/ml. Dispense 1.2 ul into each appropriate nanoplate well giving a final cell density of 2000 cells/well
   3) Agonist stock, 4 uM 9-cis RA. Dispense 0.4 ul into positive control wells giving a final agonist concentration of 1 uM.
   4) Culture time 16-20 h
   5) Dispense 0.4 ul of 5×CCF4 into each well. Load for 1.5 h.
   6) Read on tcPR.

### Example 11

### Validation of GR Nuclear Receptor Hi2h Throughput Screen

Strategy for GR Responsive Clone Isolation.

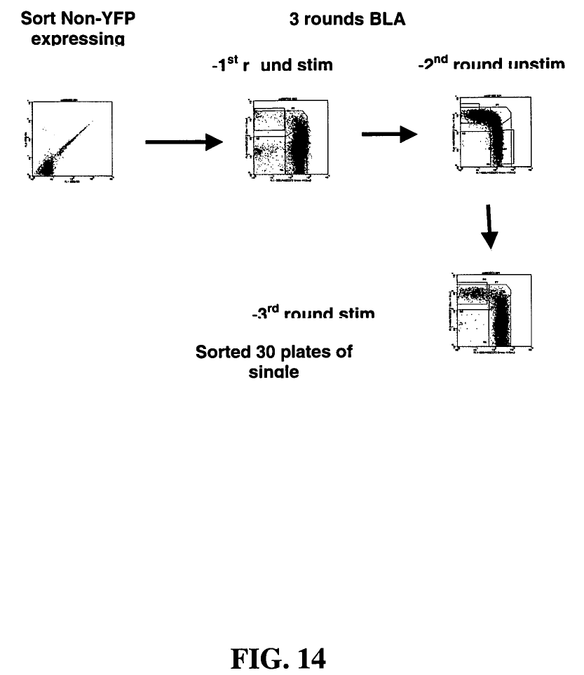

Flow cytometry was employed to isolate clones expressing active GAL4-GR chimeric receptor. The flow cytometry sorting strategy employed was as follows: first round, sort for YFP negative cells; second round, sort for dexamethasone stimulated blue cells, third round, sort for unstimulated green cells and finally a fourth round of dexamethasone stimulated blue cells (FIG. 14). This approach resulted in isolation of clones that are dependent on the functional activity of GAL4-GR.

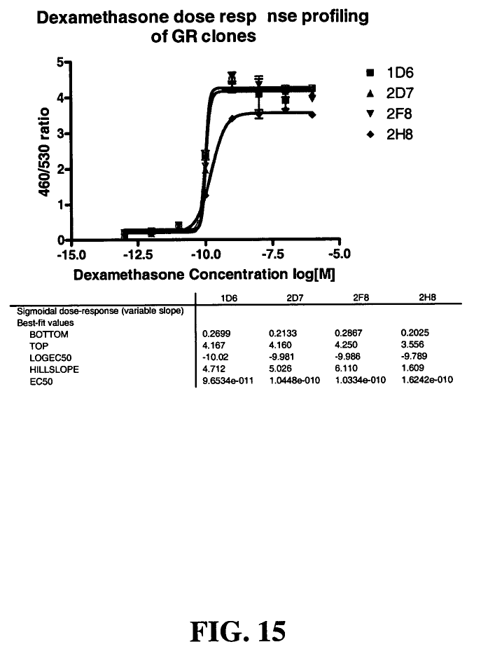

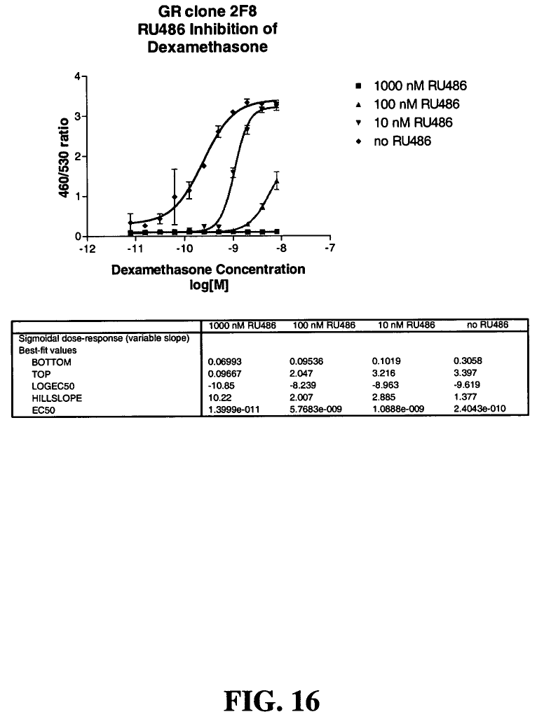

Individual HEK/UAS/GAL4-GR FACS sorted clones were expanded and quantitatively analyzed for β-lactamase expression. Dexamethasone induced a P-lactamase response in several clones with sub-nanomolar EC5Os (FIG. 15). The best performing HEK/UAS/GAL4-GR clone, 2F8, was chosen for assay optimization and validation for ultra-high throughput screening. The response to dexamethasone in clone 2F8 was completely suppressed in the presence of 1 uM of the glucocorticoid antagonist RU486 (FIG. 16). Analysis of GAL4-GR integration into the genome was verified by RT-PCR with primers specific for targeted site of integration.

Cell Culture Conditions

Passage Conditions

Harvesting/Splitting Procedure for T-225 Flasks:

1. Coat new T-225 flask with 10 ml Matrigel; set aside at room temperature.

2. Aspirate media from flasks containing cells.

3. Wash cells with 10-20 ml PBS, aspirate.

4. Add 3 ml room temperature versene, rotate flask to cover cells with versene, let stand at room temperature for 2-5 min to dissociate cells.

5. Resuspend dissociated cells in 7 ml growth medium (10 mls total in flask). Triturate cells by pipetting briskly to remove clumps.

6. Split the cells at 1:10 or 1:15 if they are really dense. Use 30 ml of media per T-225. The cells will be confluent three to four days later.

7. Cells may be split at 1:2 to 1:10 depending on need. A flask at 80-90% confluency will yield approximately 20e6 cells per T-225 (˜1-7e6/ml).

Growth Medium

DMEM (Gibco Cat #12430-054 which contains glucose, L-glut, 25 mM HEPES and pyridoxine HCl)

10% heat-inactivated and dialyzed FBS (55 mls/500 ml media)

1× Pen/Strep (5.5 ml Pen-Strep/500 ml media, Gibco cat #15140-122)
1 mM Sodium Pyruvate (5.5 ml 100 mM NaPyruvate/500 ml media, Gibco cat #11360-070)
1× Non-essential amino acids (5.5 ml NEAA/500 ml media, Gibco cat #11140-050)
   Nanoplate Assay Validation

The assay was validated in the 3456 nanoplate format. The parameters tested include but are not limited to:
1) Cell density. The range of cell density tested was from 2000 cells/well to 12000 cells/well. Optimal screening density was determined to be from 4000-6000 cells/well on the nanoplate assay. Use 4000 cells/well for nanoplate screening.
2) Culture time. Optimal culture time is between 16-24 h. Allow 16-20 h of incubation during screening.
3) Agonist Concentration. 1 uM Dexamethasone stimulated cells act as positive controls during nanoplate screening.
4) CCF4 loading time. Use 1.5 hr CCF4 loading time during screening.
5) DMSO sensitivity. The assay tolerates at least 0.7% DMSO with no change in performance.
6) Assessment of assay window in 3456 well-format with bioactive compound set Nanoplate experimental parameters used on the FRD:
   1) Assay media, DMEM, 2% CD-FBS, Pen/Strep
   2) Cell stock, 3.33e6/ml. Dispense 1.2 ul into each appropriate nanoplate well giving a final cell density of 4000 cells/well
   3) Agonist stock, 4 uM Dexamethasone. Dispense 0.4 ul into positive control wells giving a final agonist concentration of 1 uM.
   4) Culture time 16-20 h
   5) Dispense 0.4 ul of 5×CCF4 into each well. Load for 1.5 h.
   6) Read on tcPR.

### Example 12

### Validation of MR Nuclear Receptor High Throughput Screen

Isolation of Aldosterone-Responsive Clones

Flow cytometry was used to isolate clones expressing active GAL4-MR chimeric receptor. The flow cytometry sorting strategy employed successive rounds of sorting, for aldosterone-stimulated blue cell populations, and unstimulated green cell populations. This approach resulted in isolation of clones that are dependent on the functional activity of GAL4-MR.

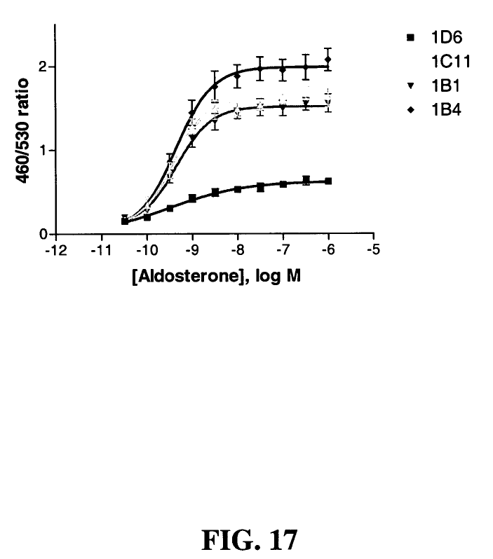

Individual HEK/UAS/GAL4-MR FACS sorted clones were expanded and quantitatively analyzed for β-lactamase expression. Several aldosterone-inducible clones were selected for further validation (FIG. 17). Analysis of GAL4-MR integration into the genome was verified by RT-PCR with primers specific for targeted site of integration. The best performing HEK/UAS/GAL4-GR clone (1B4) was chosen for assay optimization and validation for ultra-high throughput screening.

Pharmacology of MR Clone 1B4

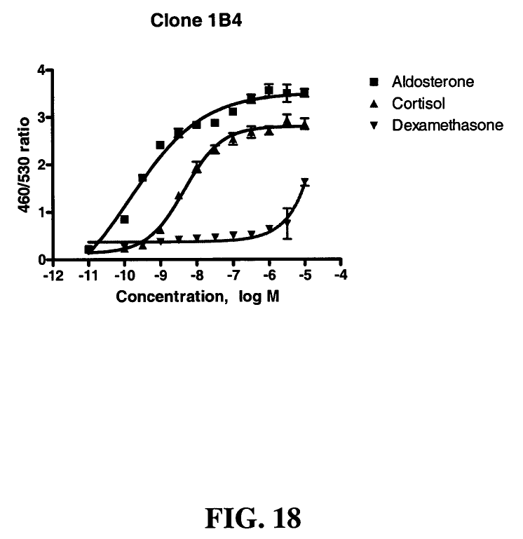

Clone 1B4 was selected for further validation due to its strong response to aldosterone and low unstimulated background. Response to aldosterone, cortisol and dexamethasone are shown in FIG. 18. The clone responds to aldosterone with an EC50 of 0.15 nM. Cortisol, which has lower but still appreciable affinity for MR had an EC50 of 4 nM, while dexamethasone was relatively inactive.

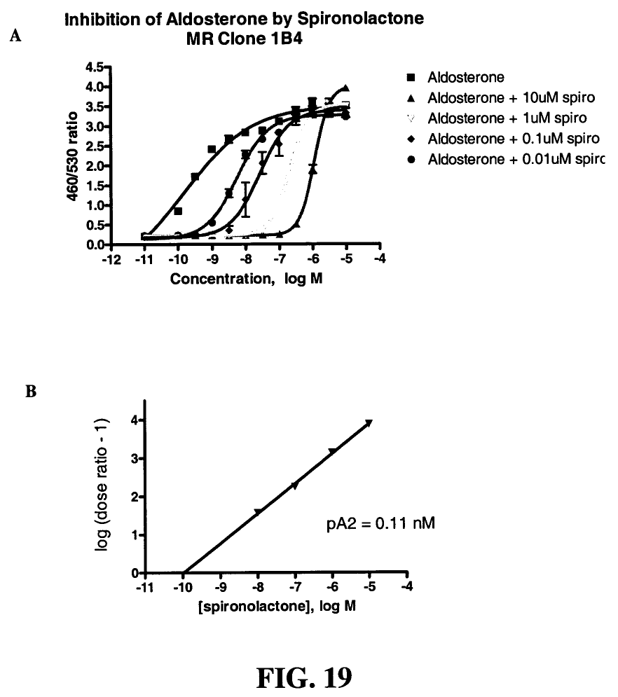

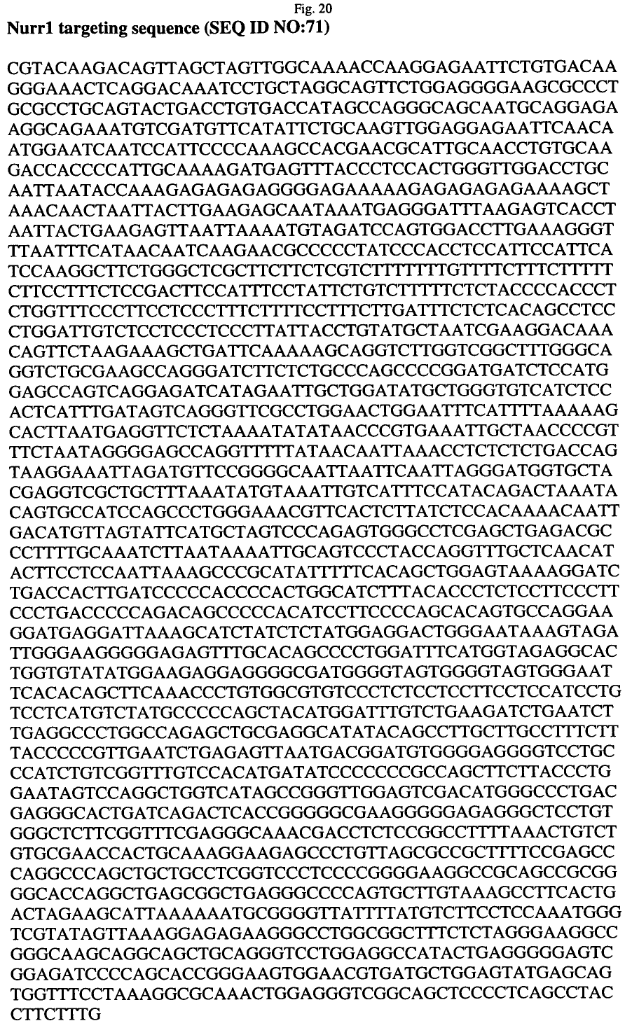

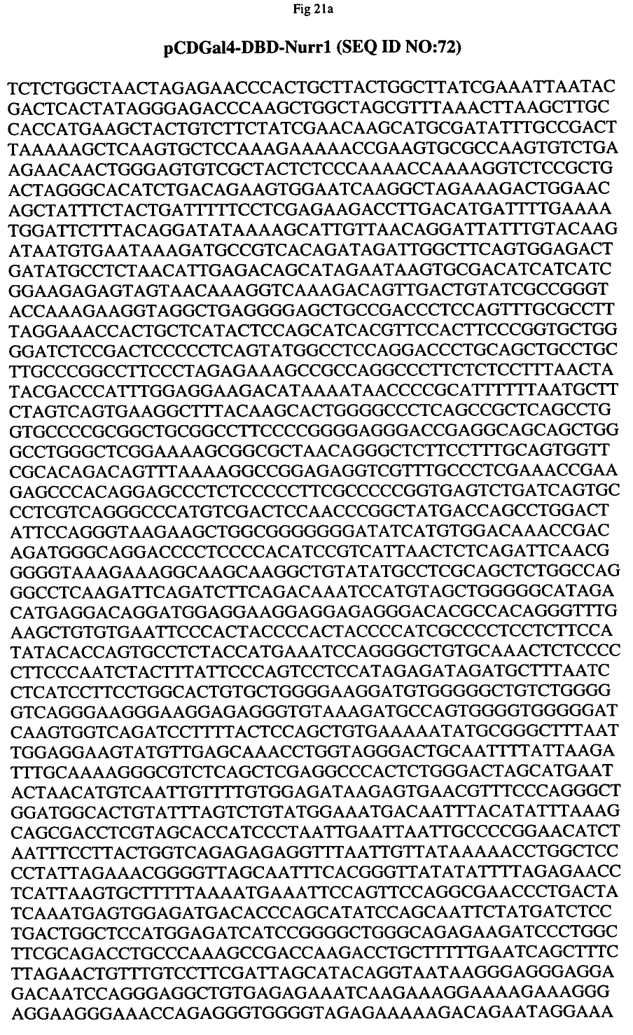

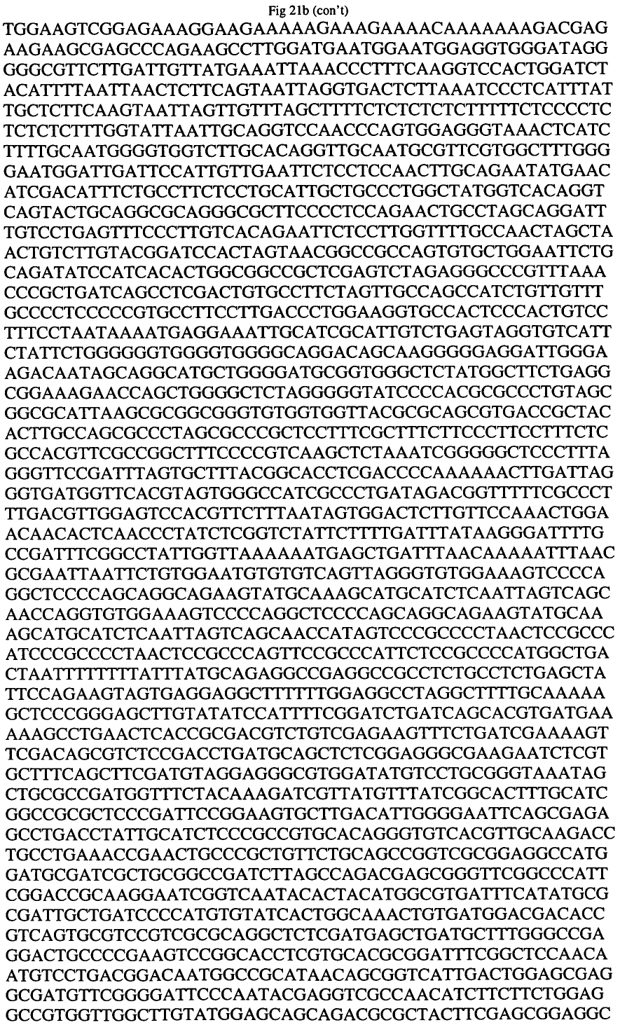

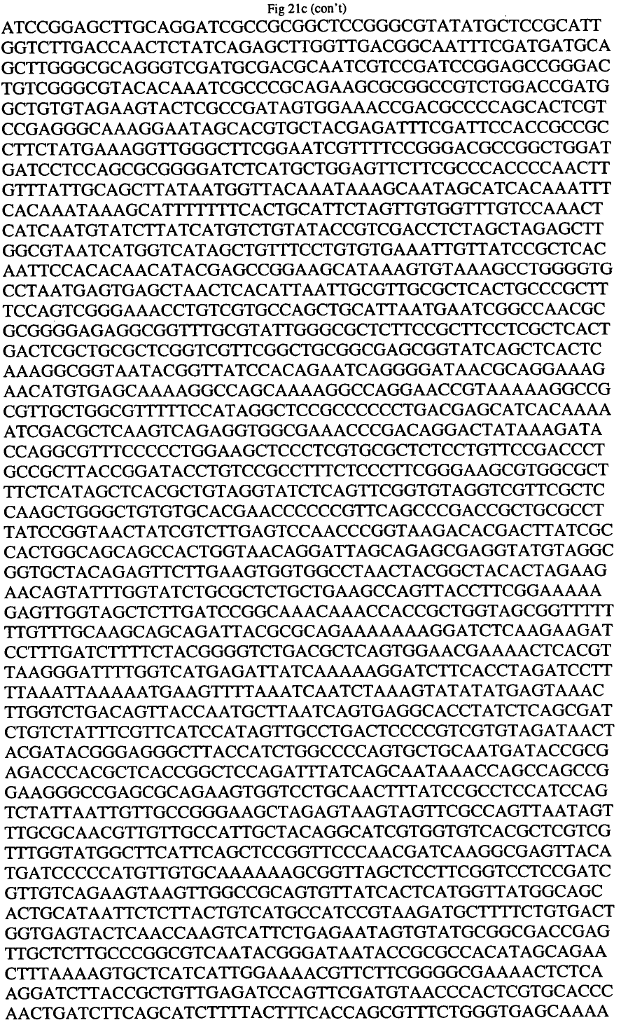

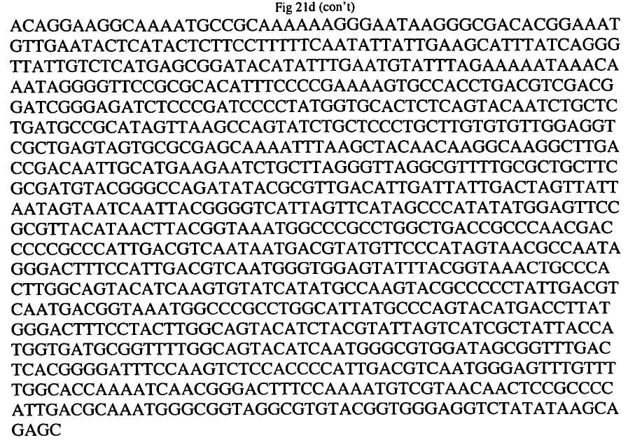

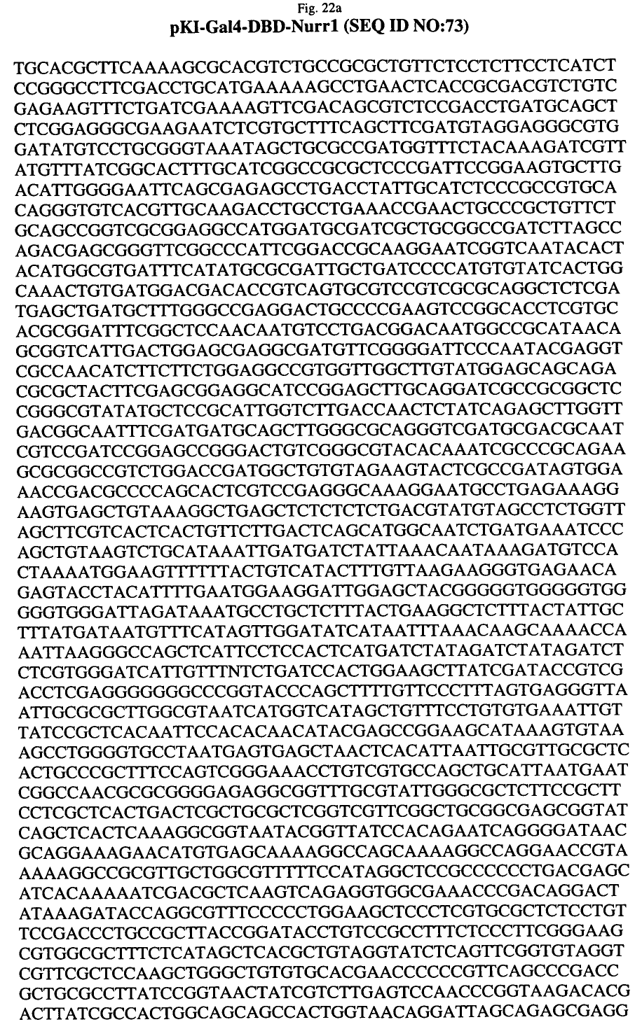

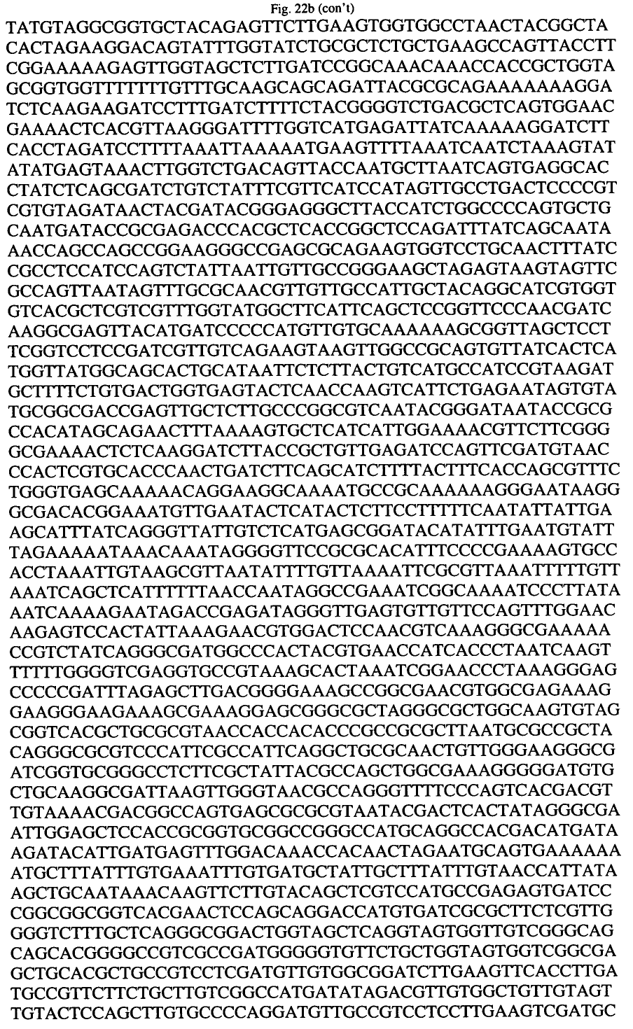

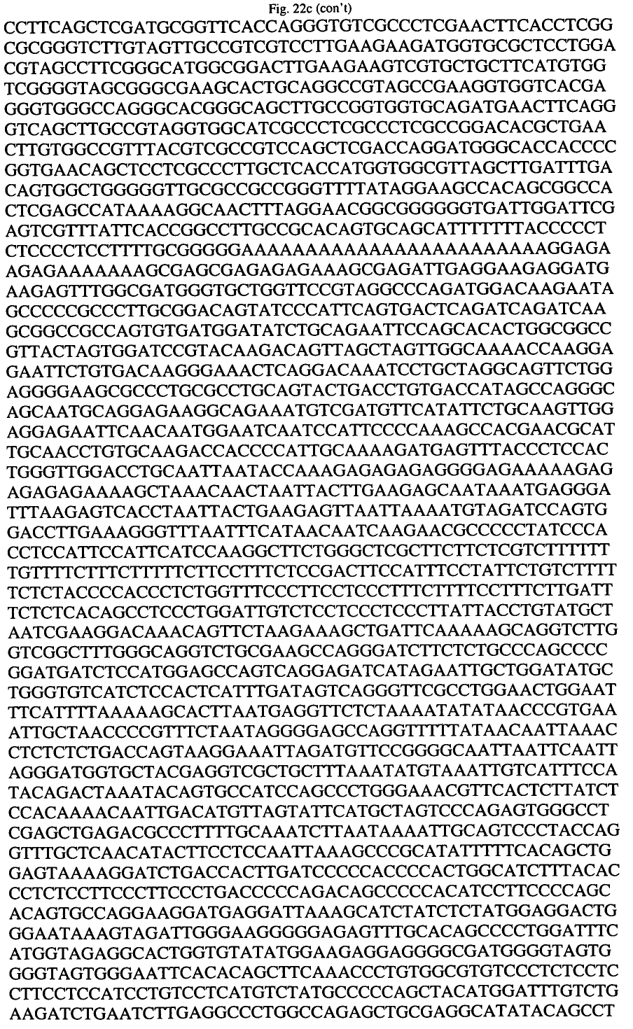

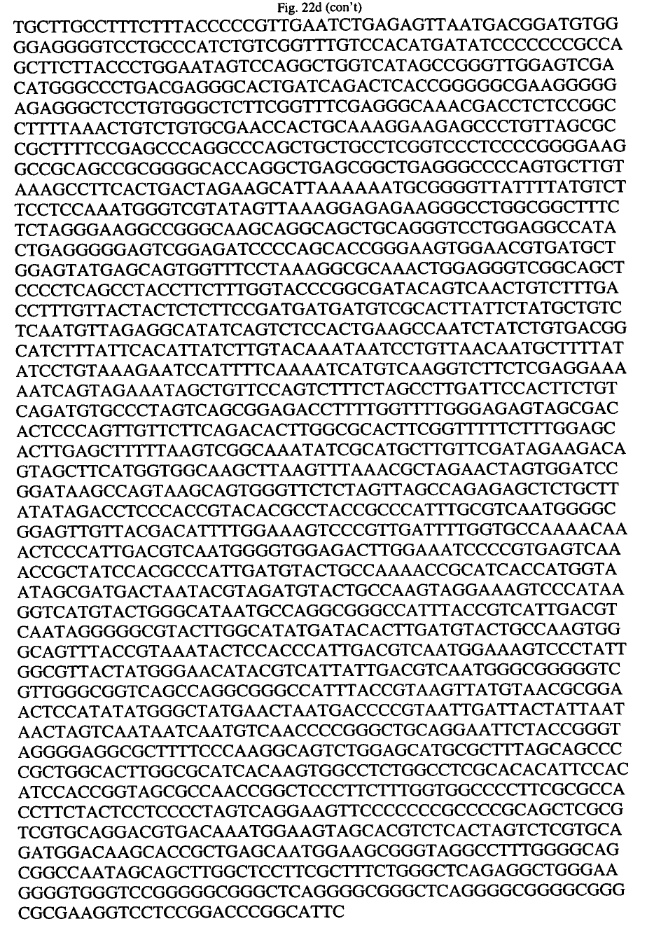

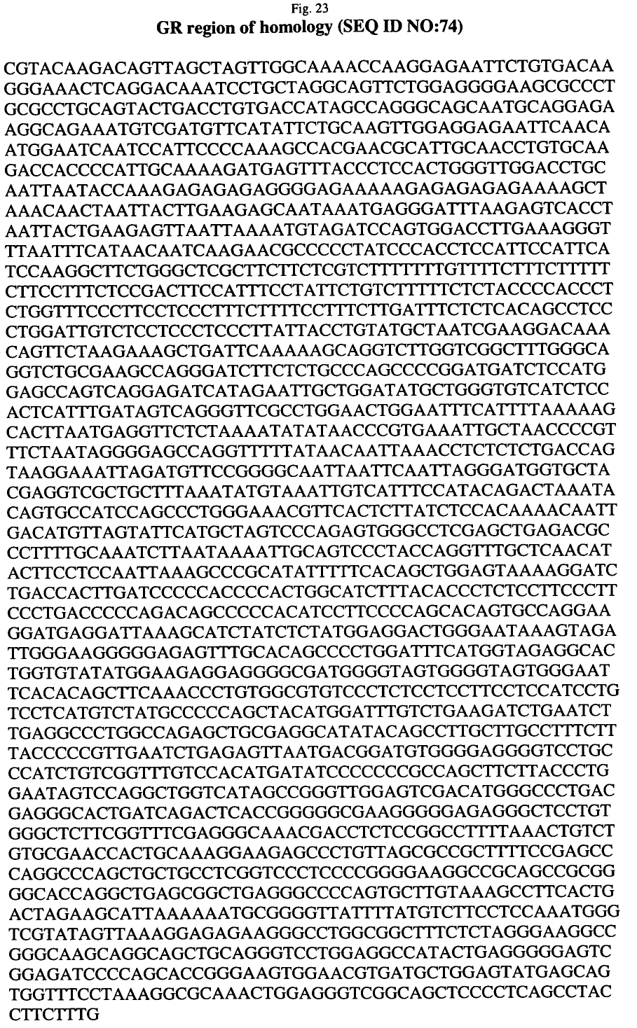

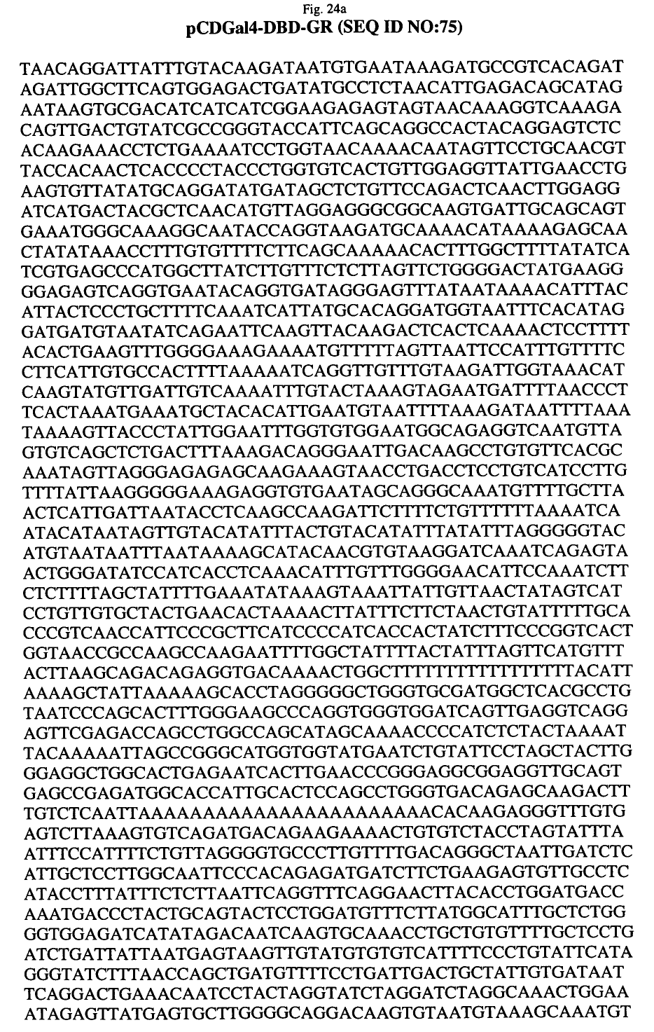

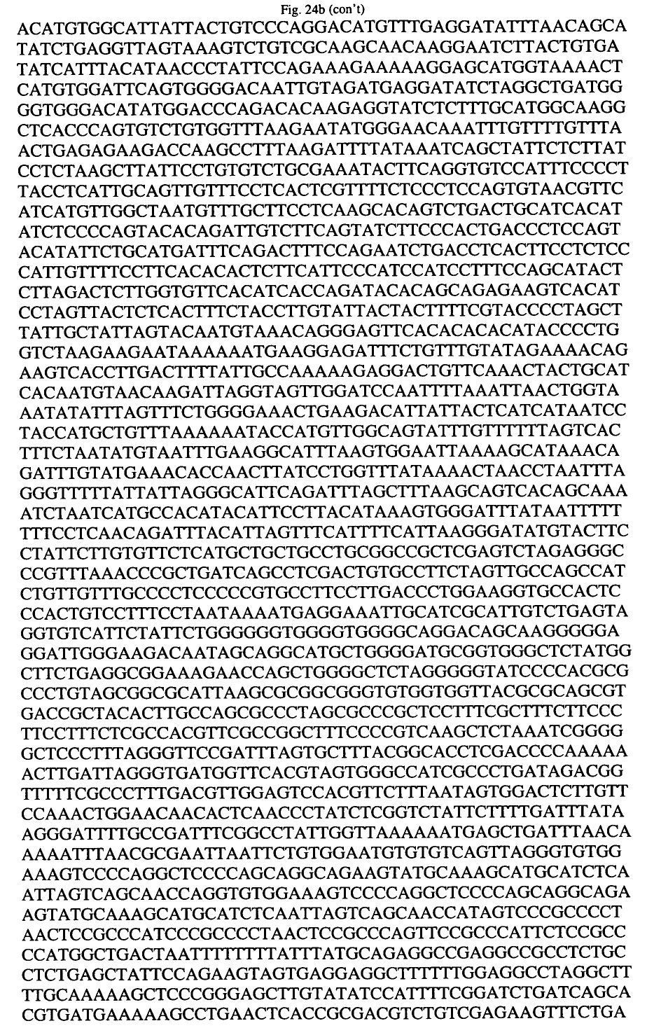

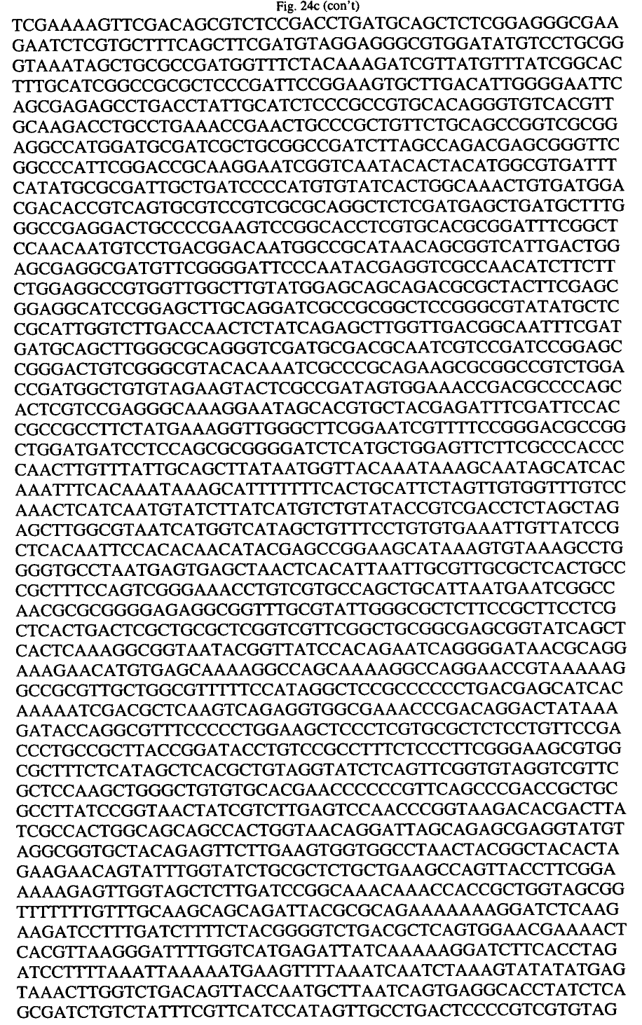

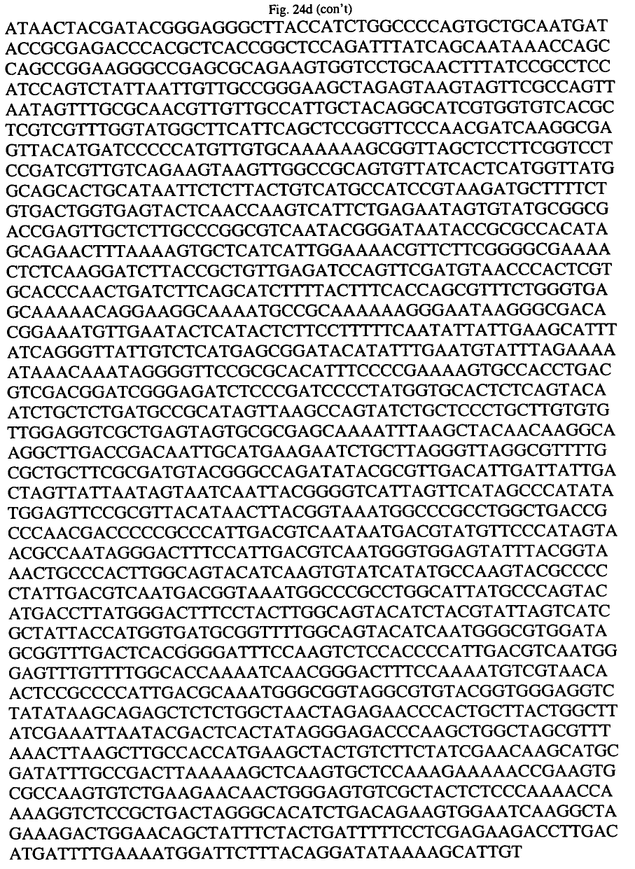

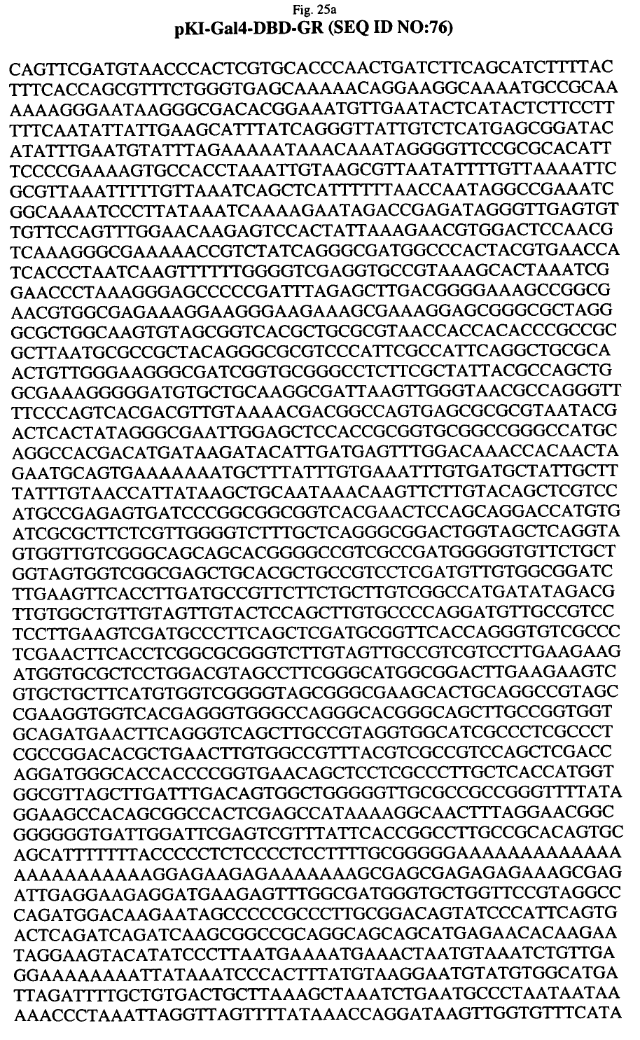

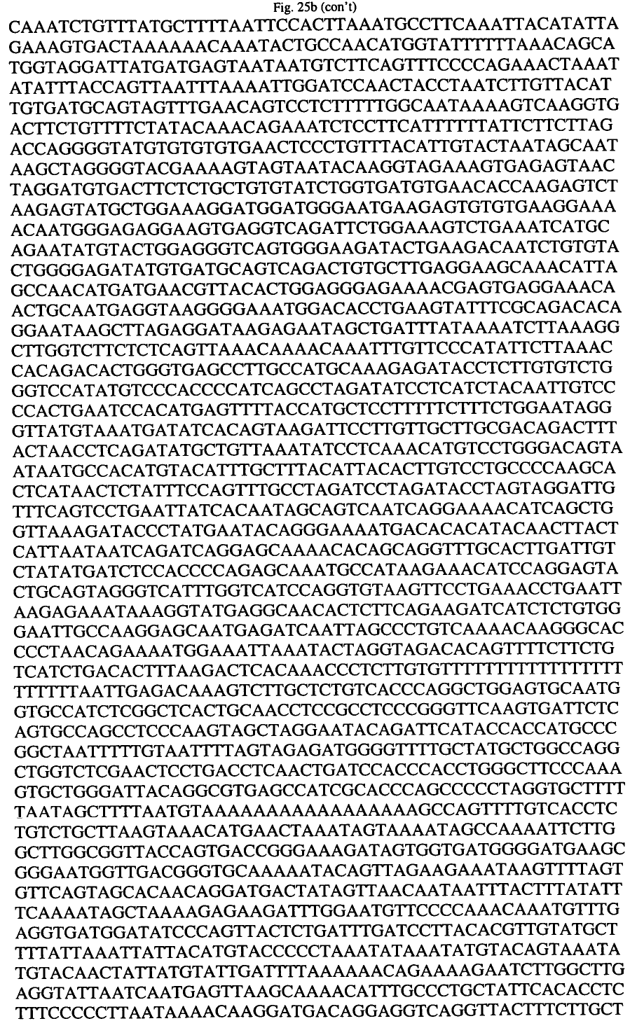

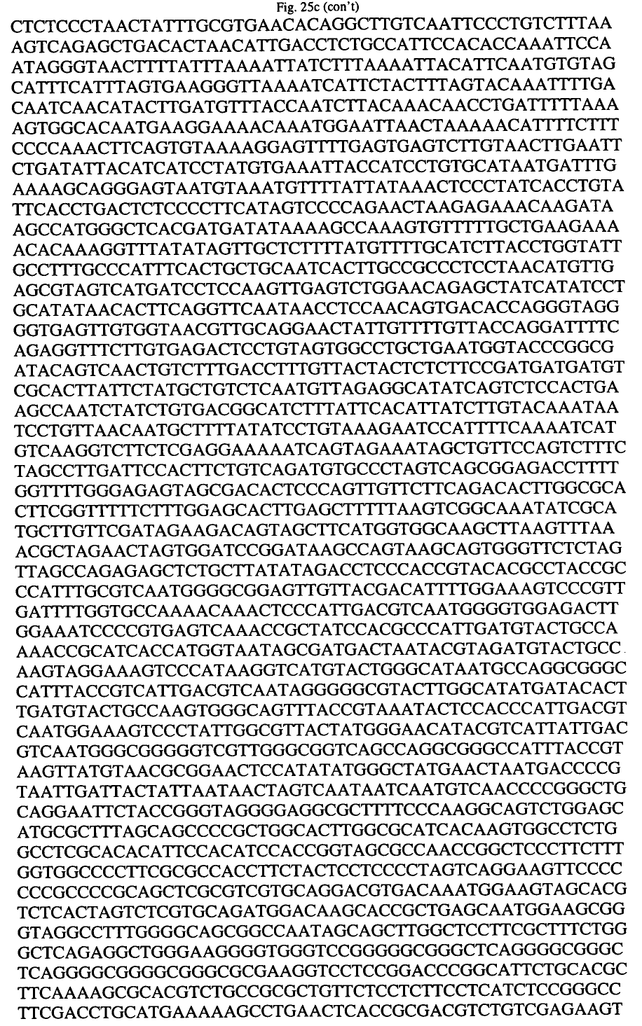

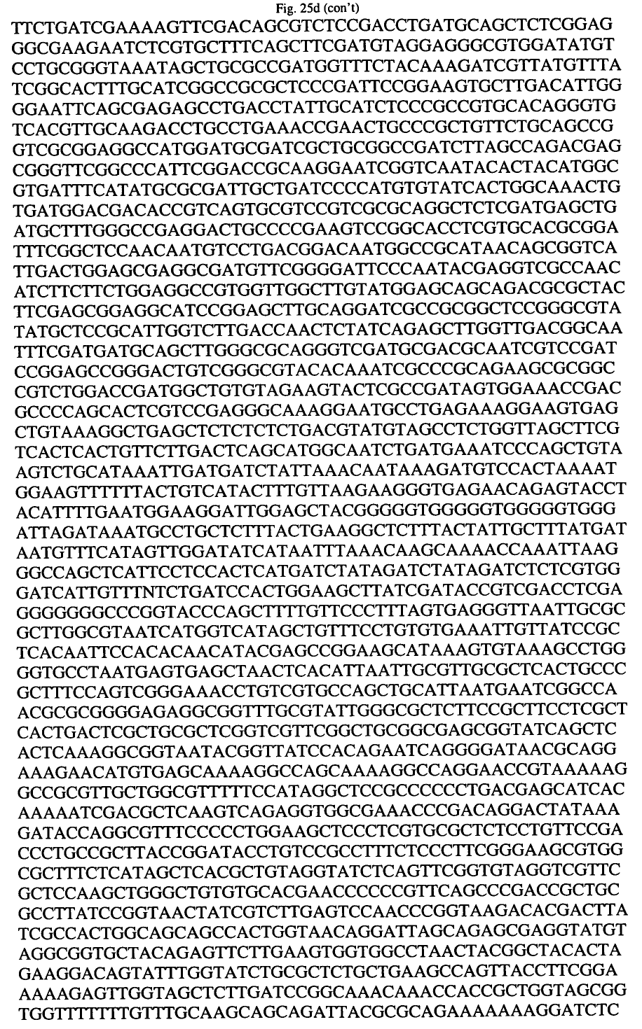

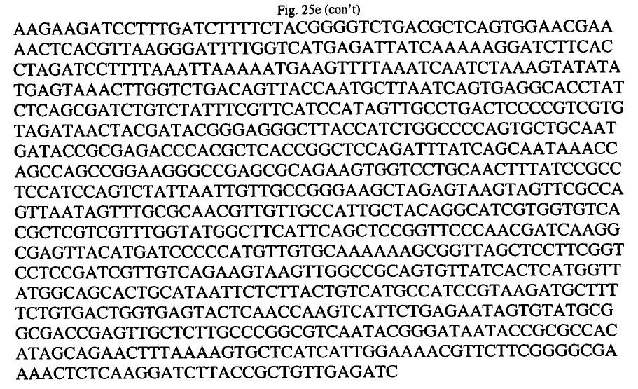

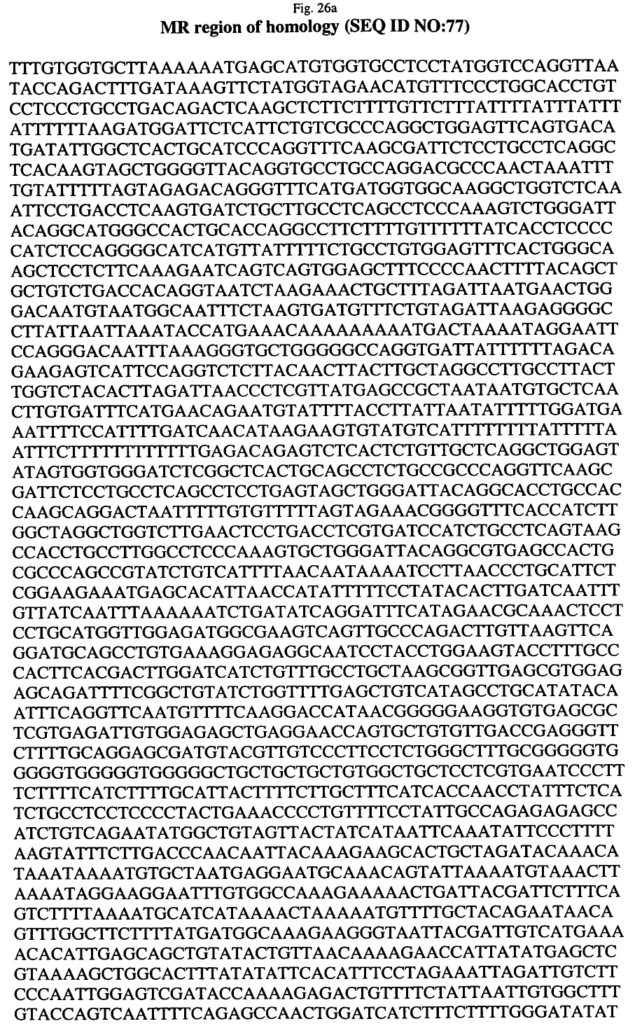

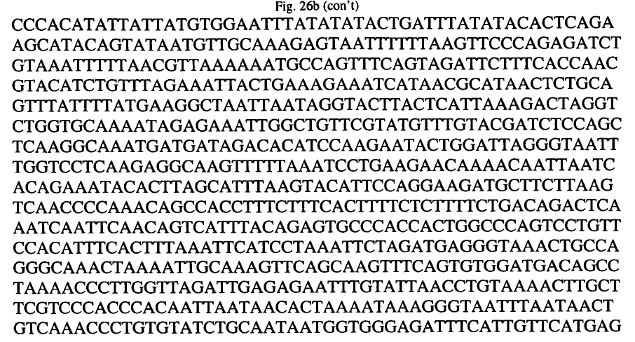

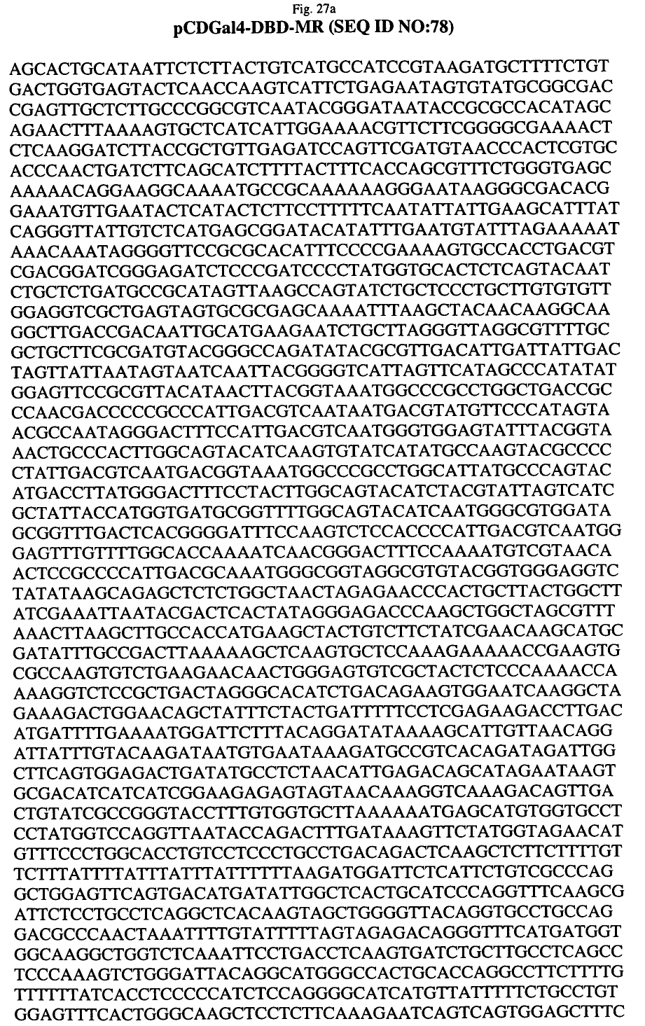

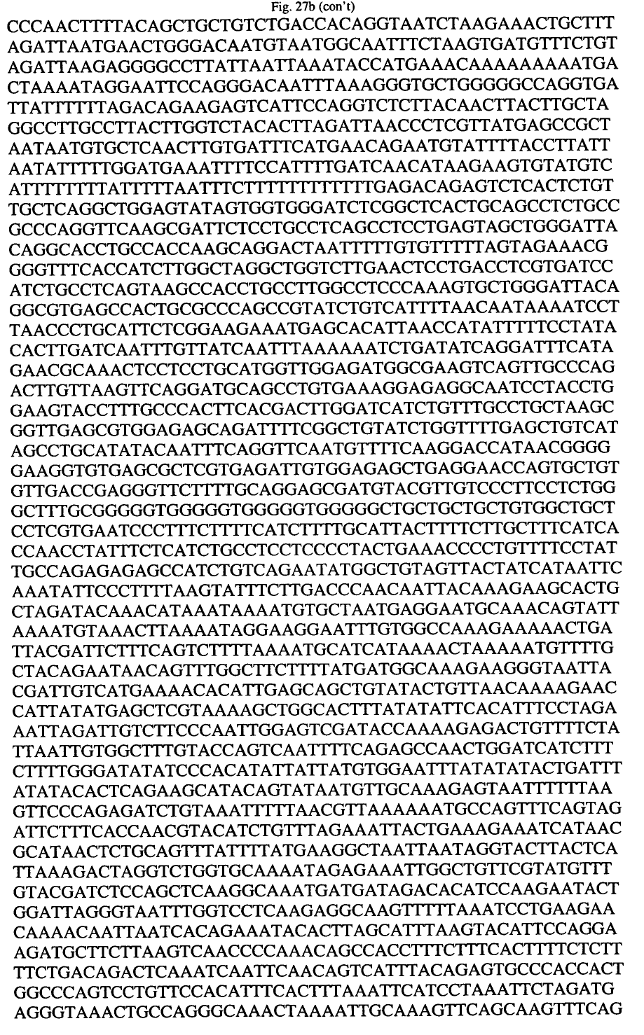

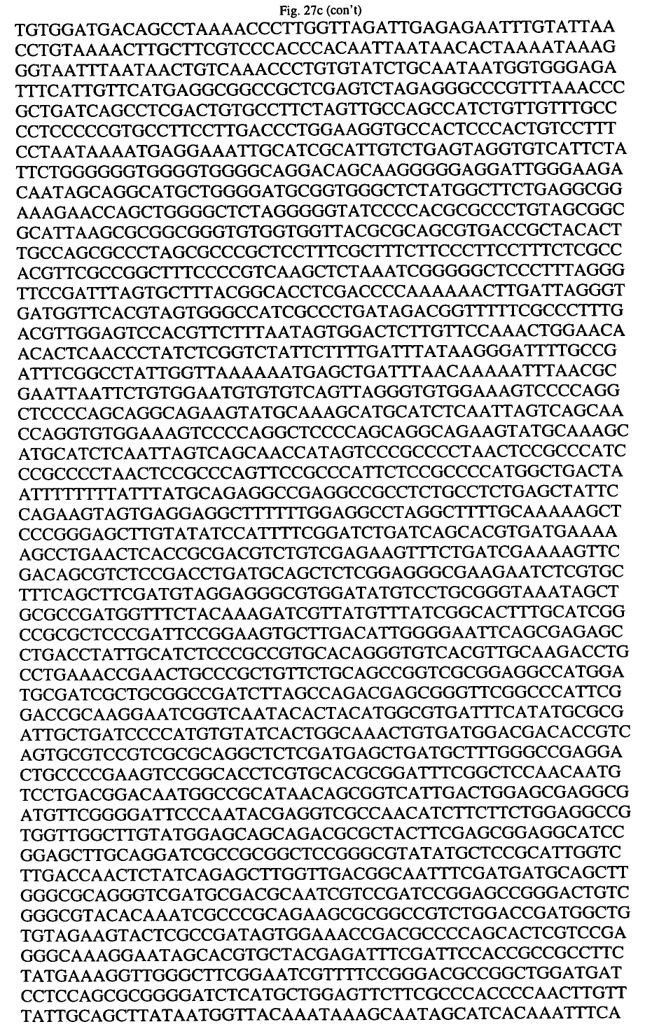

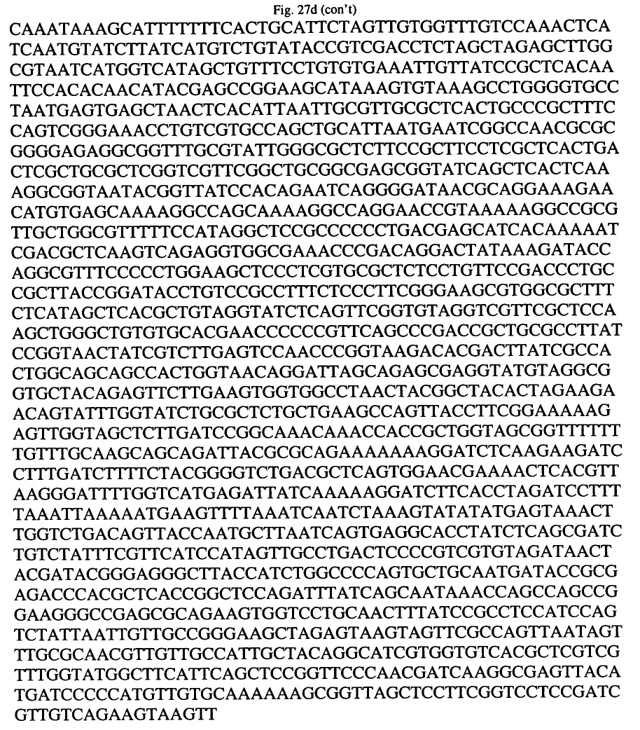

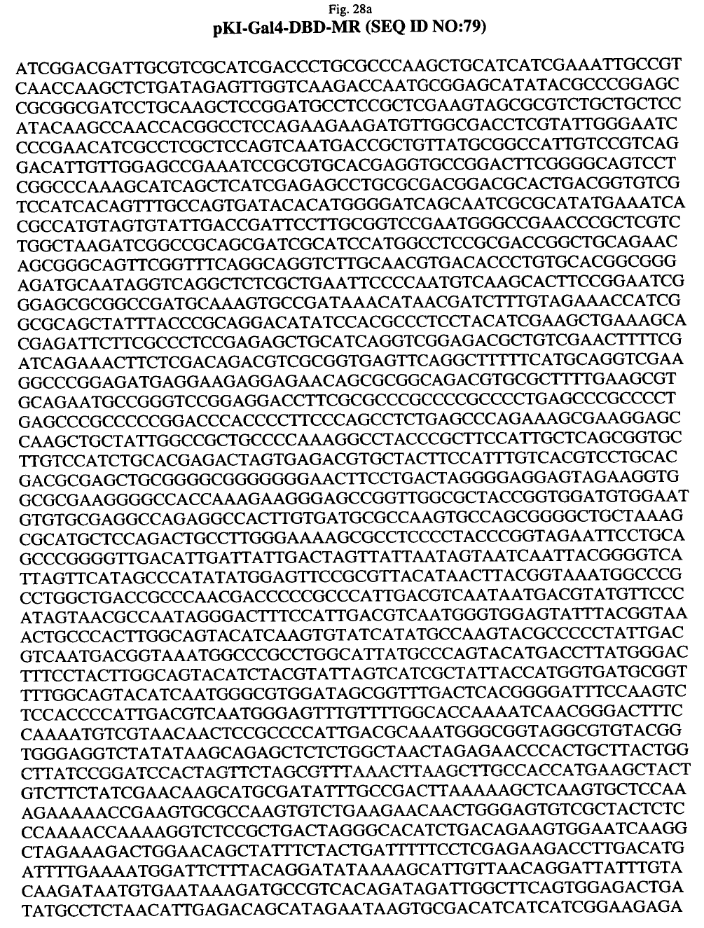

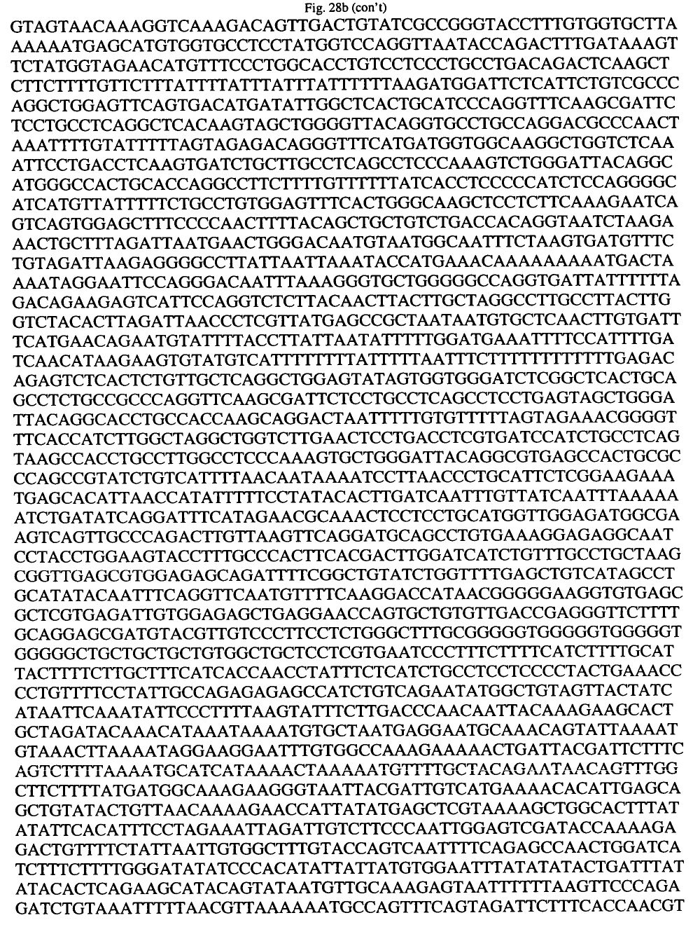

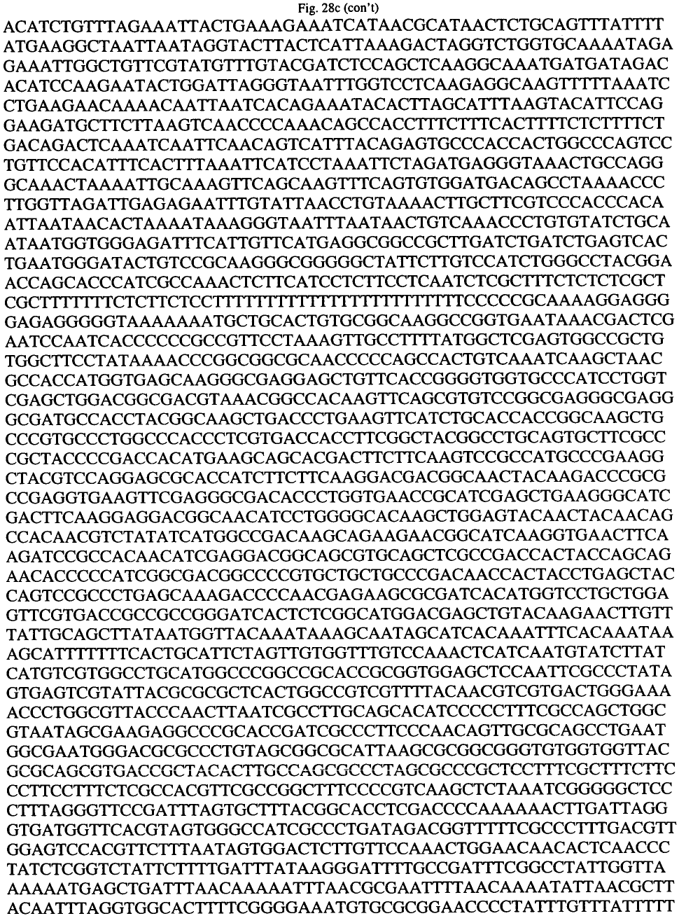

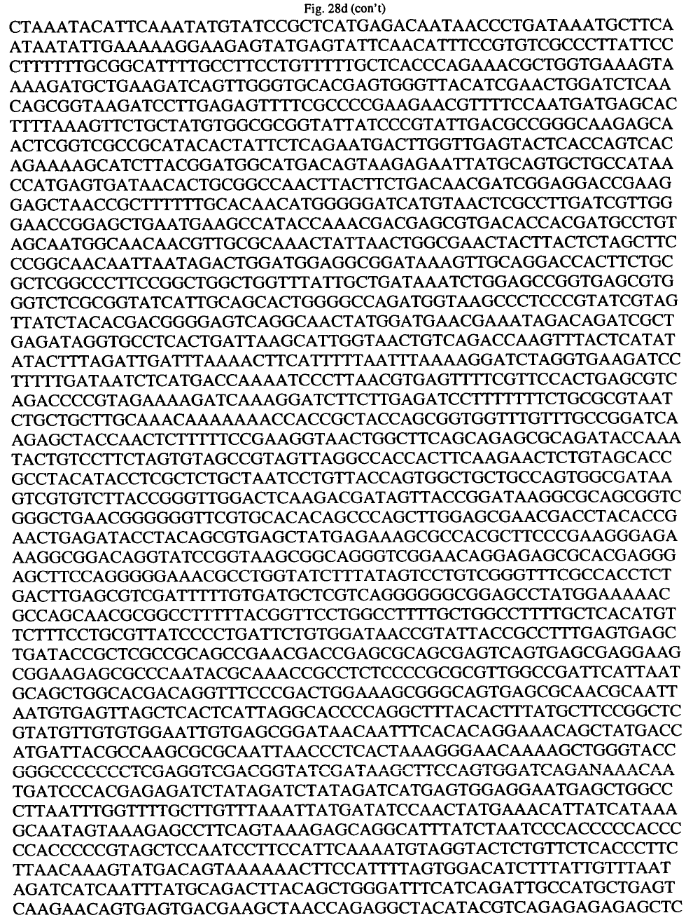

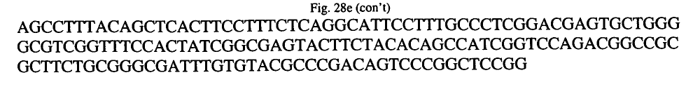

Spironolactone is a high affinity MR antagonist. The response to aldosterone in MR clone 1B4 was potently antagonized by spironolactone, with calculated pA2 of 0.11 nM (FIG. 19).

Cell Culture Conditions

Passage Conditions

Harvesting/Splitting Procedure for T-225 Flasks:

1. Coat new T-225 flask with 10 ml Matrigel; set aside at room temperature.

2. Aspirate media from flasks containing cells.

3. Wash cells with 10-20 ml PBS, aspirate.

4. Add 3 ml room temperature versene, rotate flask to cover cells with versene, let stand at room temperature for 2-5 min to dissociate cells.

5. Resuspend dissociated cells in 7 ml growth medium (10 mls total in flask). Triturate cells by pipetting briskly to remove clumps.

6. Split the cells at 1:10 or 1:15 if they are really dense. Use 30 ml of media per T-225. The cells will be confluent three to four days later.

7. Cells may be split at 1:2 to 1:10 depending on need. A flask at 80-90% confluency will yield approximately 20e6 cells per T-225 (˜1-7e6/ml).

Growth Medium

DMEM (Gibco Cat #12430-054 which contains glucose, L-glut, 25mM HEPES and pyridoxine HCl)

10% heat-inactivated and dialyzed FBS (55 mls/500 ml media)

1× Pen/Strep (5.5 ml Pen-Strep/500 ml media, Gibco cat #15140-122)

1 mM Sodium Pyruvate (5.5 ml 100 mM NaPyruvate/500 ml media, Gibco cat #11360-070)
1× Non-essential amino acids (5.5 ml NEAA/500 ml media, Gibco cat #11140-050)
   Nanoplate Assay Validation

The assay was validated in the 3456-well nanoplate format. The parameters tested include:
1) Cell density. Optimal screening density was determined to be from 4000-6000 cells/well in the nanoplate assay. Use 4000 cells/well for nanoplate screening. For screening in 384-well format, use 20,000 cells/well.
2) Culture time. Optimal culture time is between 16-24 h. Allow 16-20 h of incubation with compounds during screening.
3) Agonist concentration. 1 uM Aldosterone-stimulated cells act as positive controls during 384-well and nanoplate screening.
4) CCF4 loading time. Use 1.5 hr CCF4 loading time during screening
5) DMSO sensitivity. The performance of the assay with varying concentrations of DMSO is shown in FIG. 4. The assay performance is only slightly affected by 0.5% DMSO, and performance is good with up to 1% DMSO.
6) Assessment of assay window in 3456 well-format.

Nanoplate experimental parameters used on the FRD:
1) Assay media, DMEM, 2% Charcoal Dextran-treated-FBS, Pen/Strep
2) Cell stock, 3.33e6/ml. Dispense 1.2 ul into each appropriate nanoplate well giving a final cell density of 4000 cells/well
3) Agonist stock, 4 uM Aldostersone. Dispense 0.4 ul into positive control wells giving a final agonist concentration of 1 uM.
4) Culture time 16-20 h
5) Dispense 0.4 ul of 5×CCF4 into each well. Load for 1.5 h.
6) Read on tcPR.

### Example 13

### Vanilloid Receptor-1 (VR1) Cell Line for Antagonist Assay

Following the teachings of the present invention, a novel cell line, designated HEK-293 c5B11 VR1 ACD#411, was developed for use in a Vanilloid Receptor-1 Antagonist Assay. A deposit of the present cell line was made with the American Type Culture Collection, 10801 University Park, Manassas, Va. 20110-2209 on ______, 2003 and assigned ATCC Accession No. ______.

Expression Method:

A plasmid vector containing the targeting sequence p-KI Master-SD-Van-YFP was used to introduce the vanilloid gene fragment as well as the yellow fluorescent protein (YFP) gene fragment into the human embryonic kidney cell line, HEK-293. The targeting sequence was obtained by amplifying a 3.8 kb genomic fragment for the Vanilloid Receptor using primer pair (5′ CGATCTAGAGAGCCACACCCCATGTTGTCTCAC 3′) (SEQ ID NO:80) (Van For) and (5′ GAGCGGCCGCTTGCCAAGGGCCCTGTGAAGCAGG3′) (SEQ ID NO:81) (Van Rev) as described above. This PCR fragment comprises the intronic sequence between the 1^{st} and 2^{nd} exon of the Vanilloid genomic region (SEQ ID NO:86). The PCR fragment was cloned into pCR2.1 Topo, the sequence confirmed and placed into pKI-CMV-SD (SEQ ID NO:82) via the Xba INot I restrictions sites to make pKI-CMV-SD-Vanilloid (SEQ ID NO:83). The eYPFP (SEQ ID NO:84) expression sequence from pKI-CMV-YFP was digested with Eag I and transferred to pKI-CMV-SD-Vanilloid to produce pKI-CMV-SD-Vanilloid-YFP (SEQ ID NO:85). This vector was linearized with Sfi I prior to electroporation.

Clone Selection Method:

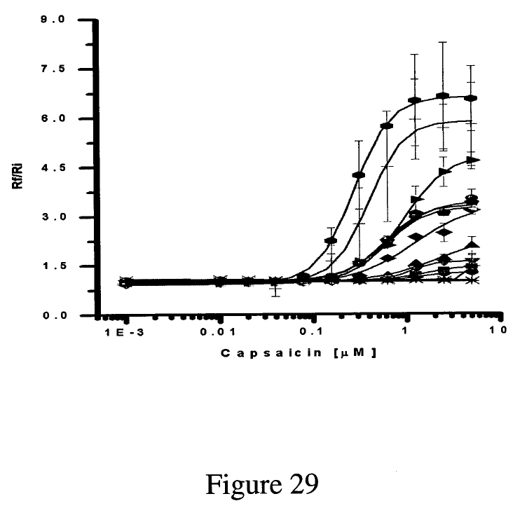

A pool of cells believed to be expressing the vanilloid receptor was obtained and a functional FACS sort was conducted whereby non-YFP expressing cells were selected for. A second round and third round of FACS sorting was based on the calcium signal following application of 10 μM capsaicin. The single cells clones sorted with FACS were further tested in a 96-well format using VIPRII. 792 clones in nine 96-well plates were tested on VIPRII and 16 were selected based on their responses to 10 μM capsaicin (FIG. 29). These 16 clones were re-tested and the two best clones, 5B11 and 5B5, were selected for further analysis. Ultimately, clone 5B11 was selected for assay development purposes as this clone exhibited the most robust response to 10 μM capsaicin.

Clone Validation:

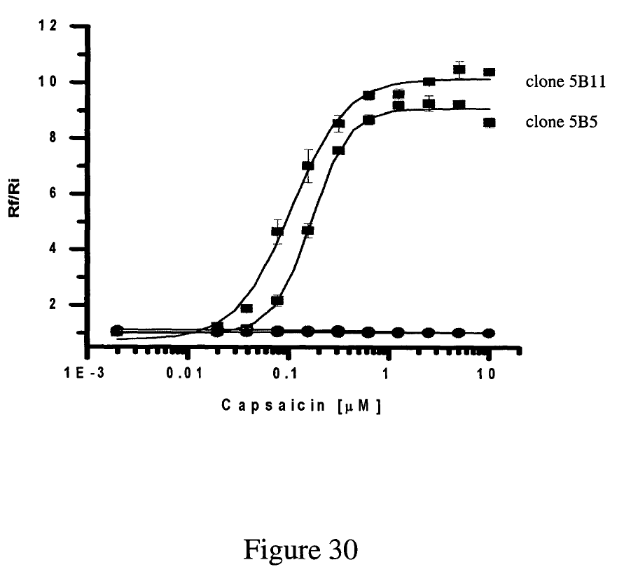

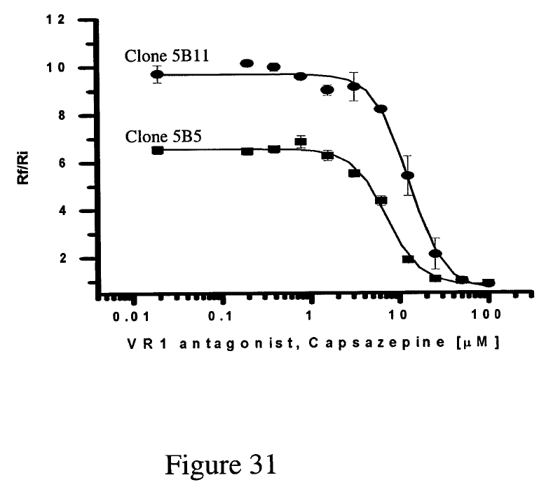

The two best clones, 5B11 and 5B5 were initially validated in 96-well format. Concentration-dependent responses to capsaicin resulted in calculated EC₅₀ values of 106 nM for clone 5B11 and 167 nM for clone 5B5. In the presence of 50 μM capsazepine, both clones failed to respond to any concentration of capsaicin (FIGS. 30 and 31). These results confirm, functionally and pharmacologically, that the HEK-293 cells are indeed expressing the vanilloid receptor type 1. Inhibitory responses to capsazepine in a concentration-dependent manner were also tested. Cells were incubated with capsazepine at concentrations between 0.01-100 μM and responses to 10 μM capsaicin were tested. The calculated IC₅₀ for capsazepine was 12.8 μM for clone 5B11 and 7.1 μM for clone 5B5 (FIGS. 30 and 31). These IC₅₀ values are in line with those reported in the literature.

Concentration-Response Curves:

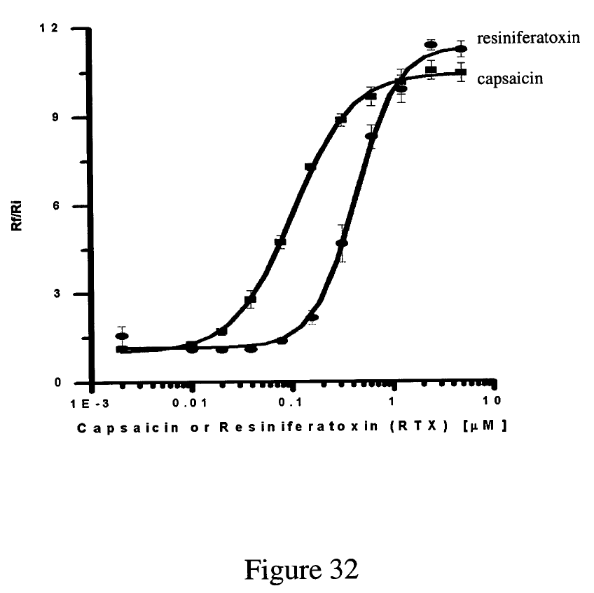

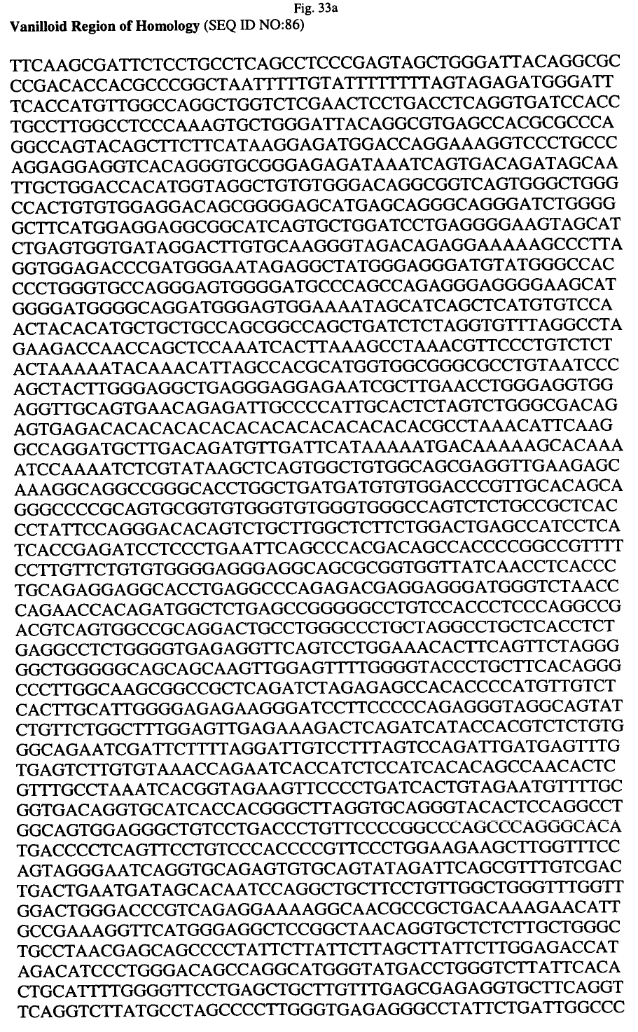

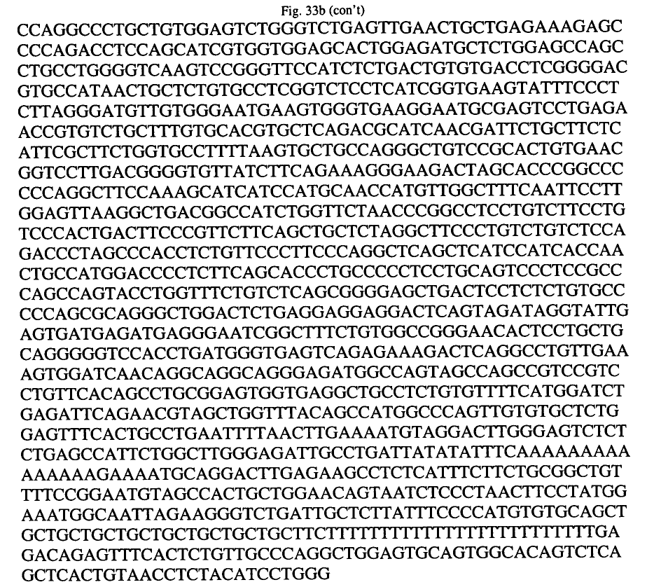

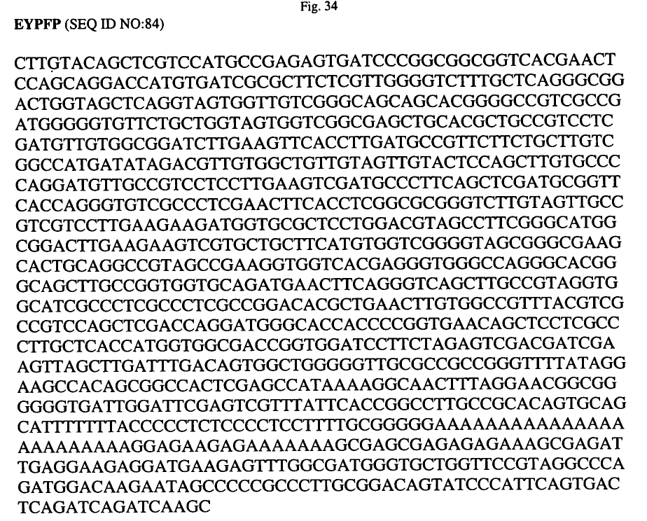

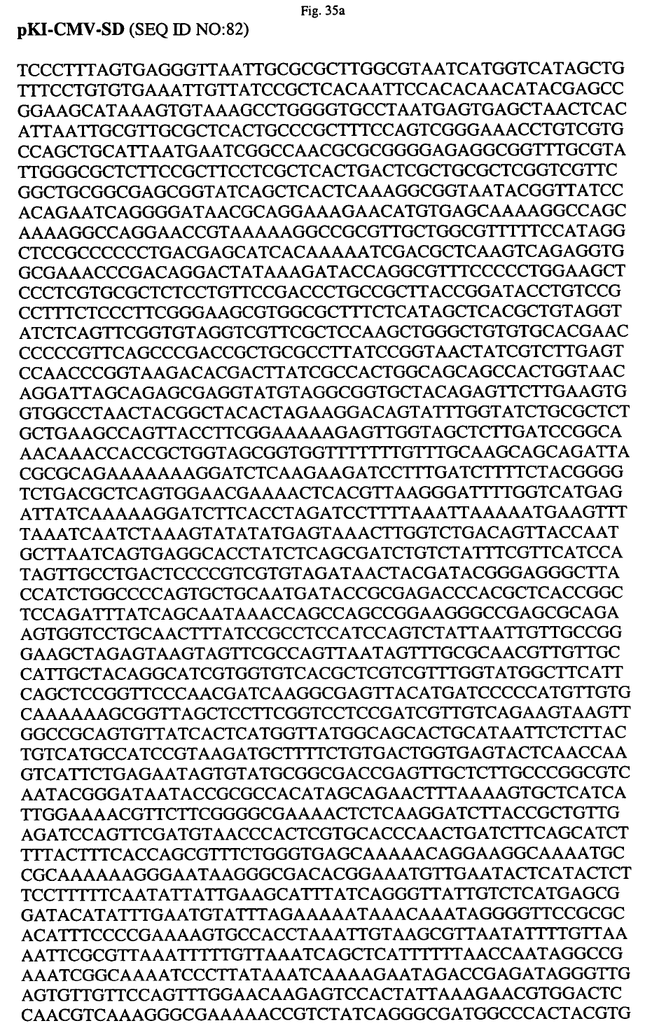

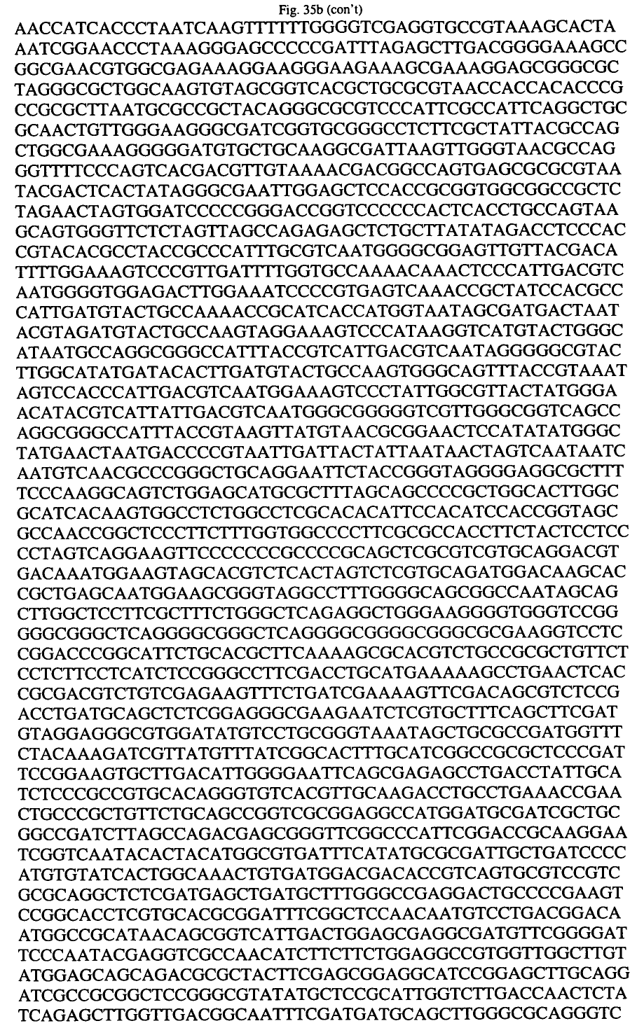

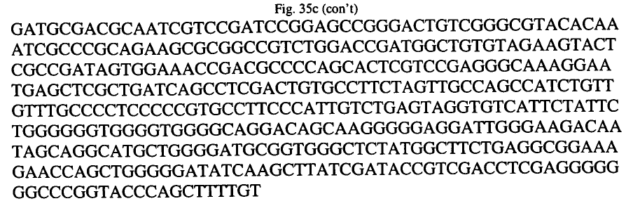

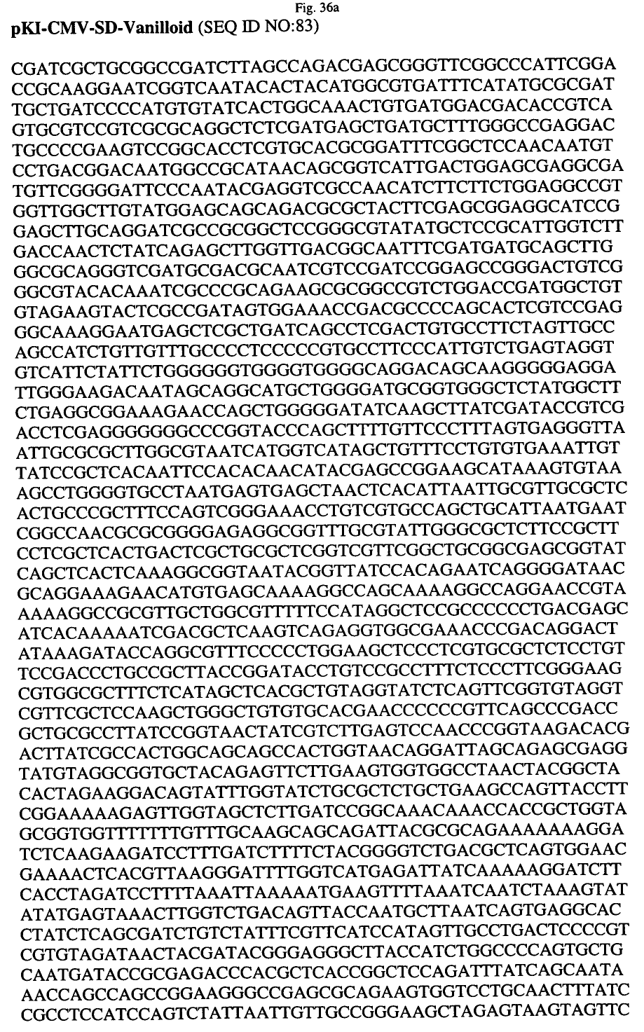

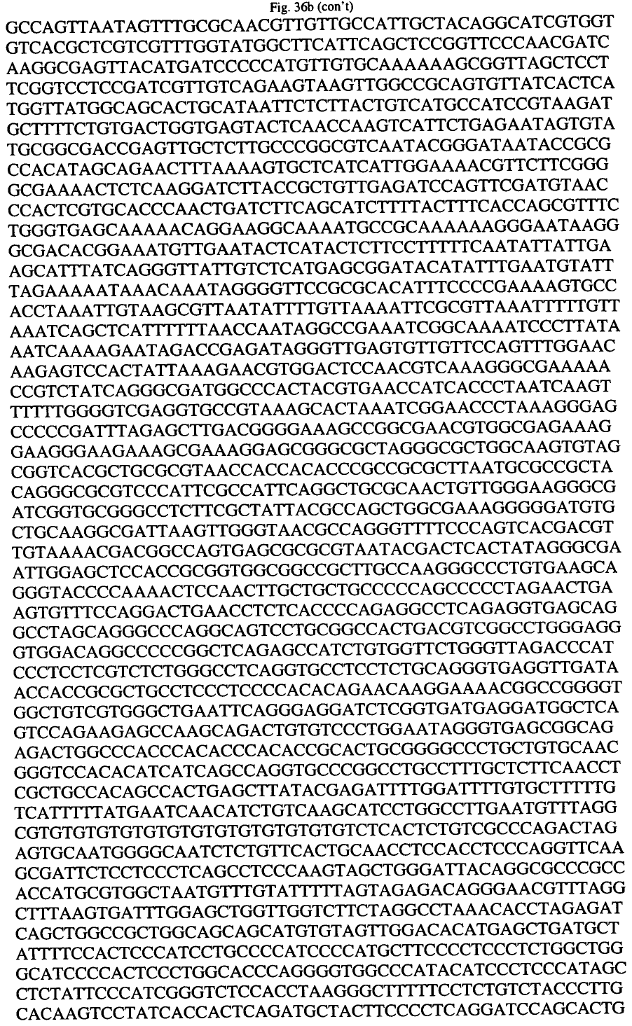

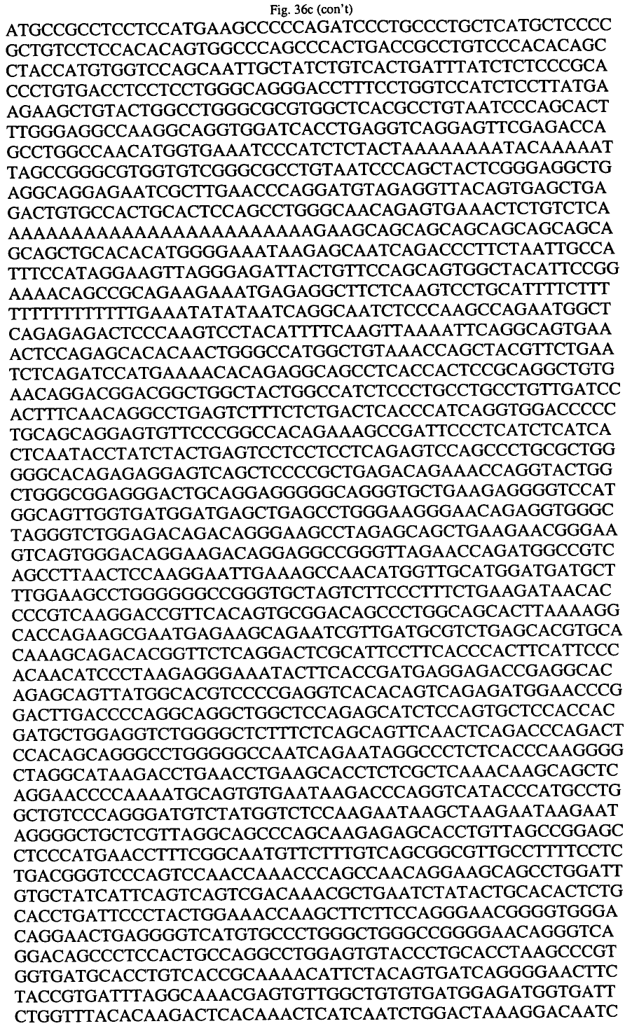

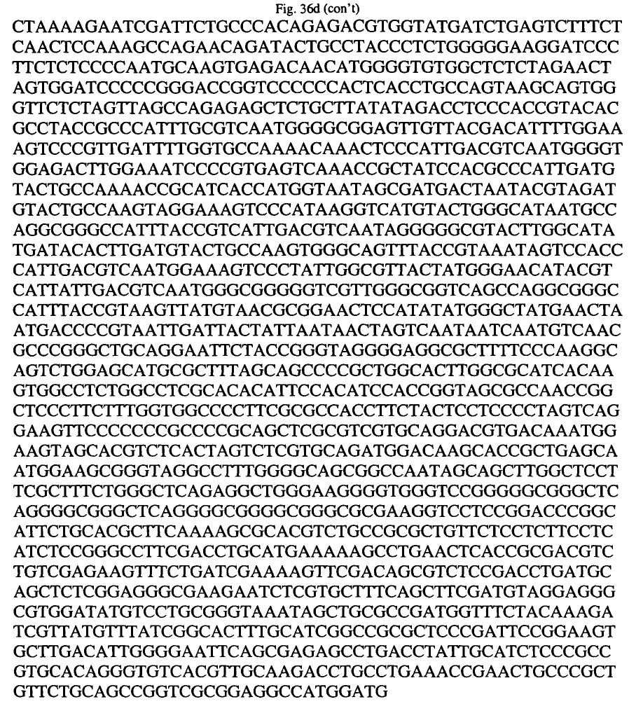

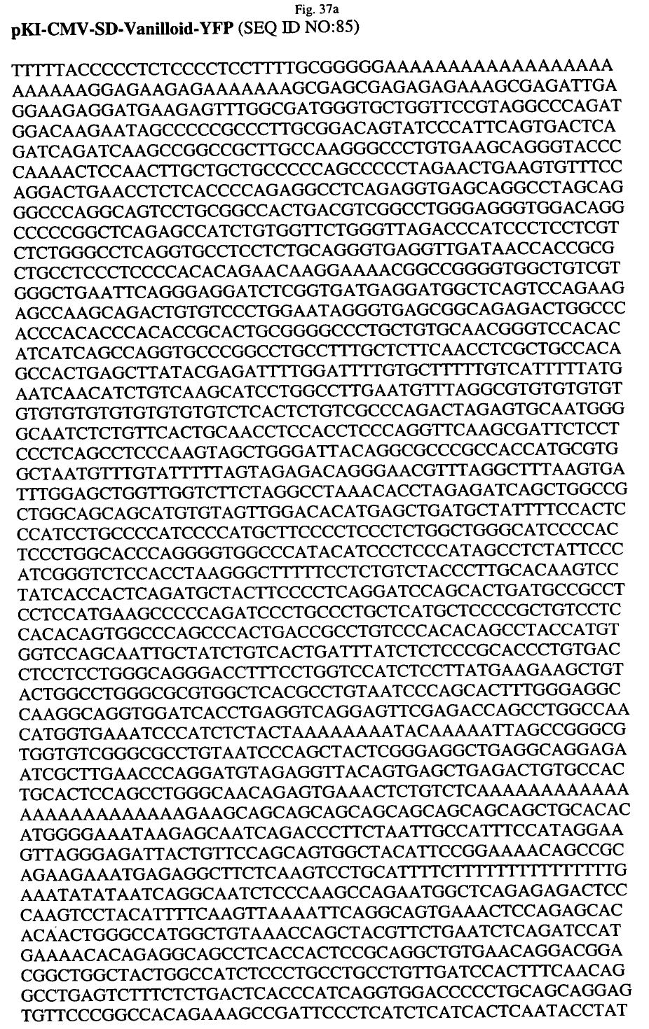

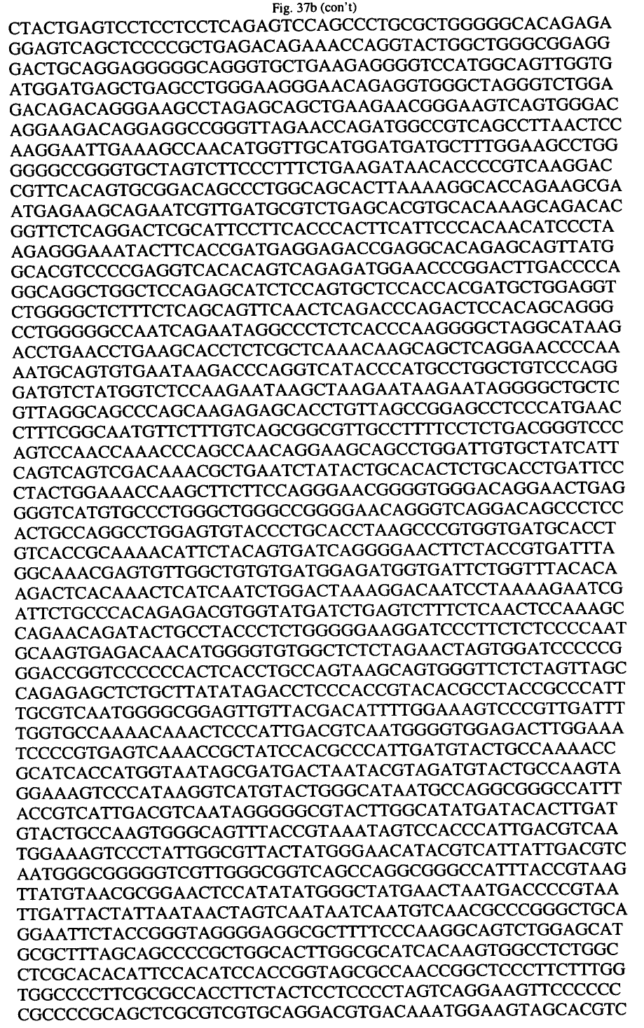

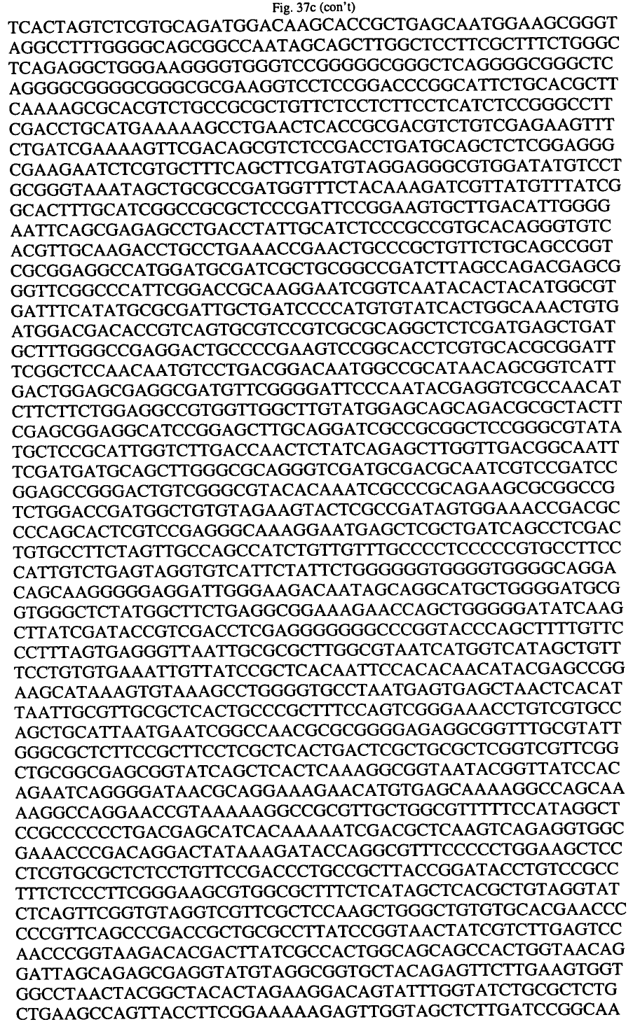

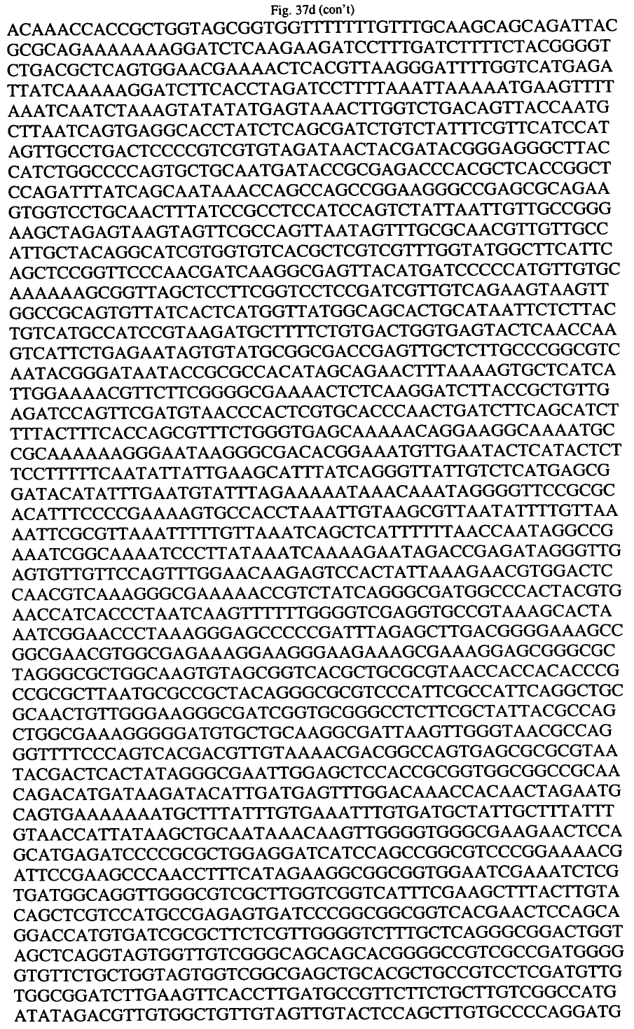

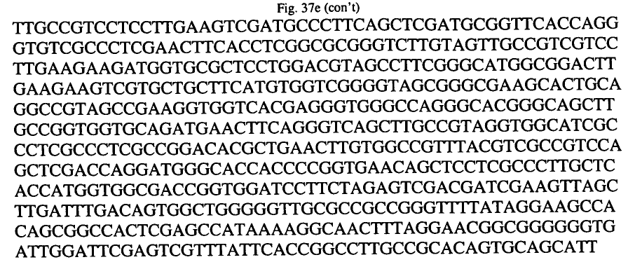

Concentration-response curves were obtained from HEK-293 cells expressing VR1 in 96-well format. Responses to two VR1 agonists were tested. The calculated EC₅₀ for capsaicin was 103 nM and 427 nM for resiniferatoxin (FIG. 32). These results are similar to values reported in published literature. The assay was further validated by testing the activity of known antagonists of the VR1 receptor. As described previously, in the presence of 50 μM capsazepine, VR1 responses to capsaicin were inhibited (FIG. 30 and 31).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention that are obvious to those skilled in the field of molecular biology or related fields are intended to be within the scope of the following claims.

1. A method of developing a sensor cell for determining the activity of a target gene in said cell, comprising the steps of:
a. providing a homogeneous population of cells, wherein each of said cells comprises a signal transduction detection system,
b. introducing into said population of cells an isolated DNA construct comprising a promoter operatively linked to a targeting sequence, wherein:
i. said targeting sequence comprises a region of homology to said target gene sufficient to promote homologous recombination of said isolated DNA following introduction into said cells;
ii. said promoter is heterologous to said target gene;
iii. following said recombination said promoter controls transcription of an mRNA that encodes a polypeptide comprising an activatable domain; and
iv. said polypeptide is capable, upon activation of said activatable domain, of altering the signal detected from said signal transduction system,
c. incubating said population of cells under conditions which cause expression of said polypeptide;
d. incubating said population of cells under conditions which cause activation of said activatable domain of said polypeptide; and
e. selecting cells that have altered the signal detected from said signal transduction system.

2. The method according to claim 1, wherein:
a. said target gene encodes a polypeptide comprising a first modulator domain;
b. said isolated DNA construct further comprises a second modulator domain heterologous to said target gene, wherein said second modulator domain is positioned in said DNA construct relative to said targeting sequence such that following homologous recombination said promoter controls the transcription on an mRNA that encodes a polypeptide comprising an activatable domain and said second modulator domain, but lacking said first modulator domain; and
c. upon activation of said activatable domain said modulator domain is capable of altering the signal detected from said signal transduction system.

3. The method according to claim 1 or 2, wherein said signal transduction detection system is selected from a reporter gene detection system, a transmembrane potential change detection system, a post-translational modification detection system and ion sensitive detection system.

4. The method according to claim 3, wherein said reporter gene detection system comprises a reporter gene selected from β-lactamase, a naturally occurring Aequorea victoria green fluorescent protein, or a mutant Aequorea victoria green fluorescent protein.

5. The method according to claim 3, wherein said transmembrane potential change detection system is a FRET-based assay.

6. The method according to claim 3, wherein said ion sensitive detection system comprises an ion sensitive fluorophore selected from an UV light-excitable calcium indicator, a low affinity calcium indicator, a visible light-excitable calcium indicator, an UV light-excitable magnesium indicator, or a visible light-excitable magnesium indicator.

7. The method according to claim 2, wherein said second modulator domain comprises a zinc finger DNA binding domain.

8. The method according to claim 2, wherein said modulator domain comprises a DNA binding domain selected from a nuclear receptor DNA binding domain, a Gal4 DNA binding domain or a LexA DNA binding domain.

9. The method according to claim 1 or 2, wherein said target gene is selected from a nuclear receptor, a G-protein subunit, an ion channel subunit or a G-protein coupled receptor.

10. The method according to claim 1 or 2, wherein said isolated DNA construct further comprises a selectable marker gene.

11. A recombinant sensor cell comprising:
a. a signal transduction detection system; and
b. a promoter operatively linked to a DNA sequence that encodes a polypeptide comprising an activatable domain, wherein:
i. said activatable domain is homologous to all or a portion of a polypeptide encoded by a target gene
ii. said promoter is heterologous to said target gene; and
iii. upon expression of said polypeptide and activation of said activatable domain the signal detected from said signal transduction detection system is altered.

12. The cell according to claim 11, wherein said polypeptide additionally comprises a modulator domain that is heterologous to said target gene, and wherein, upon expression of said polypeptide and activation of said activatable domain, said modulator domain causes the signal detected from said signal transduction system to be altered.

13. The cell according to claim 11 or 12, wherein said signal transduction detection system is selected from a reporter gene detection system, a transmembrane potential change detection system, a post-translational modification detection system and ion sensitive detection system.

14. The cell according to claim 13, wherein said reporter gene detection system comprises a reporter gene selected from β-lactamase, a naturally occurring Aequorea victoria green fluorescent protein, or a mutant Aequorea victoria green fluorescent protein.

15. The cell according to claim 13, wherein said transmembrane potential change detection system is a FRET-based assay.

16. The cell according to claim 13, wherein said ion sensitive detection system comprises an ion sensitive fluorophore selected from an UV light-excitable calcium indicator, a low affinity calcium indicator, a visible light-excitable calcium indicator, an UV light-excitable magnesium indicator, or a visible light-excitable magnesium indicator.

17. The cell according to claim 12, wherein said second modulator domain comprises a zinc finger DNA binding domain.

18. The cell according to claim 12, wherein said modulator domain comprises a DNA binding domain selected from a nuclear receptor DNA binding domain, a Gal4 DNA binding domain or a LexA DNA binding domain.

19. The cell according to claim 11 or 12, wherein said target gene is selected from a nuclear receptor, a G-protein subunit, an ion channel subunit or a G-protein coupled receptor.

20. The cell according to claim 11 or 12, further comprising a selectable marker gene.

21. A method of determining if a test compound is a modulator of a target gene or a target gene product comprising the steps of:
a. providing a recombinant sensor cell according to claim 3 or 4;
b. incubating said cell in the presence of a test compound under conditions which enable expression of said polypeptide;
c. incubating said cell under conditions which enable activation of said activatable domain of said polypeptide
d. measuring the signal detected from said signal transduction system in said cell.

22. The method according to claim 21, wherein said signal transduction detection system is selected from a reporter gene detection system, a transmembrane potential change detection system, a post-translational modification detection system and ion sensitive detection system.

23. The method according to claim 22, wherein said reporter gene detection system comprises a reporter gene selected from β-lactamase, a naturally occurring Aequorea victoria green fluorescent protein, or a mutant Aequorea victoria green fluorescent protein.

24. The method according to claim 22, wherein said transmembrane potential change detection system is a FRET-based assay.

25. The method according to claim 22, wherein said ion sensitive detection system comprises an ion sensitive fluorophore selected from an UV light-excitable calcium indicator, a low affinity calcium indicator, a visible light-excitable calcium indicator, an UV light-excitable magnesium indicator, or a visible light-excitable magnesium indicator.

26. The method according to claim 21, wherein said polypeptide comprises a zinc finger DNA binding domain that is heterologous to said target gene.

27. The method according to claim 21, wherein said polypeptide comprises DNA binding domain that is heterologous to said target gene and is selected from a nuclear receptor DNA binding domain, a Gal4 DNA binding domain or a LexA DNA binding domain.

28. The method according to claim 21, wherein said target gene is selected from a nuclear receptor, a G-protein subunit, an ion channel subunit or a G-protein coupled receptor.

29. A method of determining the activity of a target gene product comprising the steps of:
a. providing a recombinant sensor cell according to claim 3 or 4;
b. incubating said cell under conditions which enable expression of said polypeptide;
c. incubating said cell under conditions which enable activation of said activatable domain of said polypeptide
d. measuring the signal detected from said signal transduction system in said cell.

30. The method according to claim 29, wherein said signal transduction detection system is selected from a reporter gene detection system, a transmembrane potential change detection system, a post-translational modification detection system and ion sensitive detection system.

31. The method according to claim 30, wherein said reporter gene detection system comprises a reporter gene selected from β-lactamase, a naturally occurring Aequorea victoria green fluorescent protein, or a mutant Aequorea victoria green fluorescent protein.

32. The method according to claim 30, wherein said transmembrane potential change detection system is a FRET-based assay.

33. The method according to claim 30, wherein said ion sensitive detection system comprises an ion sensitive fluorophore selected from an UV light-excitable calcium indicator, a low affinity calcium indicator, a visible light-excitable calcium indicator, an UV light-excitable magnesium indicator, or a visible light-excitable magnesium indicator.

34. The method according to claim 29, wherein said polypeptide comprises a zinc finger DNA binding domain that is heterologous to said target gene.

35. The method according to claim 29, wherein said polypeptide comprises DNA binding domain that is heterologous to said target gene and is selected from a nuclear receptor DNA binding domain, a Gal4 DNA binding domain or a LexA DNA binding domain.

36. The method according to claim 29, wherein said target gene is selected from a nuclear receptor, a G-protein subunit, an ion channel subunit or a G-protein coupled receptor.

37. An isolated DNA construct comprising a promoter operatively linked to a DNA sequence which encodes a targeting sequence and a modulator domain, wherein:
a. each of said promoter, targeting sequence and modulator domain are heterologous to one another;
b. said targeting sequence comprises a region of homology to an endogenous target gene sufficient to promote homologous recombination of said DNA construct; and
c. said modulator domain is positioned in said DNA construct with respect to said targeting sequence, such that following said homologous recombination said promoter controls transcription of an mRNA encoding a polypeptide comprising an activatable domain and a modulator domain.

38. The DNA construct according to claim 37, wherein said modulator domain comprises a zinc finger DNA binding domain.

39. The DNA construct according to claim 37, wherein said modulator domain comprises a DNA binding domain selected from a nuclear receptor DNA binding domain, a Gal4 DNA binding domain or a LexA DNA binding domain.

40. The DNA construct according to claim 37, wherein said target gene is selected from a nuclear receptor, a G-protein subunit, an ion channel subunit or a G-protein coupled receptor.

41. The DNA construct according to claim 37, further comprising a selectable marker gene.

42. A recombinant cell line designated as HEK-293 MC4 c49 P4 ACD#12591 with ATCC Accession No. 5409.

43. A recombinant cell line designated as HEK-293 PPAR γ c4G5 P9 ACD#13607 with ATCC Accession No. 5405.

44. A recombinant cell line designated as HEK-293 GR c2F8 P5 ACD#13609 with ATCC Accession No. 5407.

45. A recombinant cell line designated as HEK-293 MR c1B4 P5 ACD#13687 with ATCC Accession No. 5408.

46. A recombinant cell line designated as HEK-293 Nurr1 c1E10 P7 ACD#13608 with ATCC Accession No. 5406.
